(19) United States
(12) Patent Application Publication
(10) Pub. No.: US 2006/0269614 A1
(43) Pub. Date: Nov. 30, 2006

(54) Multi-cell polarizer systems for hyperpolarizing gases

(76) Inventors: Ian Nelson, Cottage Grove, WI (US); Stephen Kadlecek, Philadelphia, PA (US); John Nouls, Durham, NC (US); Edward Karwacki, Garner, NC (US); Kenneth Bolam, Cary, NC (US); Jim Davidson, Flagstaff, AZ (US)

Correspondence Address: GE HEALTHCARE, INC., IP DEPARTMENT, 101 CARNEGIE CENTER, PRINCETON, NJ, 08540-6231, US

(21) Appl. No.: 10/542,293

(22) PCT Filed: Jan. 20, 2004
(86) PCT No.: PCT/US04/01264

§ 371 Date: May 25, 2006

(30) Foreign Application Priority Data
Jan. 17, 2003 (US) 60440747

(51) Int. Cl. H01J 27/00; A61K 33/00

(52) U.S. Cl. 424/600; 250/424

(57) ABSTRACT

Methods, systems, assemblies, computer program products and devices produce hyperpolarized gas by: (a) providing a plurality of cells (30), each having a respective quantity of target gas held therein; (b) polarizing the target gas in and/or from the cells in a desired order to provide separate batches of polarized gas; and (c) repolarizing the previously polarized target gas held in least one of the cells when the polarization level falls below a predetermined value.

### RELATED APPLICATIONS

This application claims priority to U.S. Provisional Patent Application No. 60/440,747, filed Jan. 17, 2003, the entire contents of which are hereby incorporated by reference as if fully disclosed herein.

### FIELD OF THE INVENTION

The present invention relates to the production of polarized noble gases used in NMR and magnetic resonance imaging (“MRI”) applications.

### BACKGROUND OF THE INVENTION

It has been discovered that polarized inert noble gases can produce improved MRI images of certain areas and regions of the body that have heretofore produced less than satisfactory images in this modality. Polarized helium-3 (“³He”) and xenon-129 (“¹²⁹Xe”) have been found to be particularly suited for this purpose. Unfortunately, as will be discussed further below, the polarized state of the gases is sensitive to handling and environmental conditions and can, undesirably, decay from the polarized state relatively quickly.

Hyperpolarizers are used to produce and accumulate polarized noble gases. Hyperpolarizers artificially enhance the polarization of certain noble gas nuclei (such as ¹²⁹Xe or ³He) over the natural or equilibrium levels, i.e., the Boltzmann polarization. Such an increase is desirable because it enhances and increases the MRI signal intensity, allowing physicians to obtain better images of the substance in the body. See U.S. Pat. Nos. 5,545,396; 5,642,625; 5,809,801; 6,079,213, and 6,295,834; the disclosures of these patents are hereby incorporated by reference herein as if recited in full herein.

In order to produce the hyperpolarized gas, the noble gas is typically blended with optically pumped alkali metal vapors such as rubidium (“Rb”). These optically pumped metal vapors collide with the nuclei of the noble gas and hyperpolarize the noble gas through a phenomenon known as “spin-exchange.” The “optical pumping” of the alkali metal vapor is produced by irradiating the alkali-metal vapor with circularly polarized light at the wavelength of the first principal resonance for the alkali metal (e.g., 795 nm for Rb). Generally stated, the ground state atoms become excited, then subsequently decay back to the ground state. Under a modest magnetic field (10 Gauss), the cycling of atoms between the ground and excited states can yield nearly 100% polarization of the atoms in a few microseconds. This polarization is generally carried by the lone valence electron characteristics of the alkali metal. In the presence of non-zero nuclear spin noble gases, the alkali-metal vapor atoms can collide with the noble gas atoms in a manner in which the polarization of the valence electrons is transferred to the noble-gas nuclei through a mutual spin flip “spin-exchange.”

Generally stated, as noted above, conventional hyperpolarizers include an optical pumping chamber held in an oven and in communication with a laser source that is configured and oriented to transmit circularly polarized light into the optical pumping chamber during operation. The hyperpolarizers may also monitor the polarization level achieved at the polarization transfer process point, i.e., at the optical cell or optical pumping chamber. In order to do so, typically a small “surface” NMR coil is positioned adjacent the optical pumping chamber to excite and detect the gas therein and thus monitor the level of polarization of the gas during the polarization-transfer process. See U.S. Pat. No. 6,295,834 for further description of polarization monitoring systems for optical pumping cells and polarizers.

In any event, it is now known that on-board hyperpolarizer monitoring equipment no longer requires high-field NMR equipment, but instead can use low-field detection techniques to perform polarization monitoring for the optical cell at much lower field strengths (e.g., 1-100 G) than conventional high-field NMR techniques. This lower field strength allows correspondingly lower detection equipment operating frequencies, such as 1-400 kHz. More recently, Saam et al. has proposed a low-frequency NMR circuit expressly for the on-board detection of polarization levels for hyperpolarized ³He at the optical chamber or cell inside the temperature-regulated oven that encloses the cell. See Saam et al., Low Frequency NMR Polarimeter for Hyperpolarized Gases, Jnl. of Magnetic Resonance 134, 67-71 (1998), the contents of which are hereby incorporated by reference as if recited in full herein. Others have used low-field NMR apparatus for on-board polarization measurement.

Polarizing the target gas using spin exchange optical pumping is a relatively slow process: it can take about 10 hours for a 1 liter batch of gas to reach or approach its saturation polarization. After the spin-exchange has been completed, the hyperpolarized gas is typically separated from the alkali metal prior to administration to a patient (to form a non-toxic pharmaceutically acceptable product). Unfortunately, during production and/or during and after collection, the hyperpolarized gas can deteriorate or decay relatively quickly (lose its hyperpolarized state) and therefore must be handled, collected, transported, and stored carefully.

As medical demands for polarized gas increase, there is a need for methods and systems that can provide increased volume production of polarized gas to meet production demands in a manner that can provide a reliable supply of polarized gas that is available at desired use times to facilitate hospital or clinical scheduling of associated equipment (MRI or NMR systems).

### SUMMARY OF THE INVENTION

In view of the foregoing, embodiments of the present invention provide hyperpolarizers, systems, methods, and computer program products to provide useful doses of polarized gas “on-demand”.

It is an additional object of the present invention to provide an automated hyperpolarizer that can produce multiple amounts of polarized gas in various selectable containers with varying levels of polarization decay.

It is another object of the present invention to provide systems and methods that can produce, store, and re-polarize target gases if the polarization level warrants such repolarization.

It is yet another object of the present invention to provide compact polarizer units with reduced footprint requirements that can polarize and dispense gas in clinic facilities.

It is an additional object of the present invention to provide cells and/or mounting configurations that can hold target gas in a polarizer system in a magnetic holding field.

These and other objects are satisfied by the present invention by hyperpolarizer systems that can produce a plurality of selectively polarizable amounts (batches) of target gas and other related methods, computer program products and devices.

Particular embodiments of the present invention are directed to methods for producing hyperpolarized gas. The methods include: (a) employing, or otherwise providing, a plurality of cells, each having a respective quantity of target gas held therein; (b) polarizing (serially and/or two or more concurrently) the target gas in and/or from the cells in a desired order to provide separate batches of polarized gas; and (c) repolarizing the previously polarized target gas held in at least one of the cells.

The method can further include monitoring the polarization level of each of the batches of polarized target gas in the cells during a monitoring period and directing the repolarizing step when the polarization level falls below a predetermined value.

In certain embodiments, the polarized target gas in at least one of the cells has a different polarization decay cycle relative to the polarized target gas in the other cells during a monitoring period. That is, the gas within the cells is decaying over time. However, the gas in at least one of the cells may not be decaying as it can be repolarized while polarization decay is occurring in other cells (so its polarization is increasing). The different cells can each have different decay cycles, and/or a different time at which its polarization reaches an undesirably low level.

Other embodiments are directed to methods for providing hyperpolarized noble gas including: (a) employing, or otherwise providing, a substantially cylindrical solenoid to generate a magnetic holding field, the solenoid having an elongate cavity and an associated axial center line, orienting the solenoid so that the axial center line extends at an angularly offset direction with vertical and horizontal components; and (b) holding a quantity of polarized gas in the cavity of the solenoid.

The method may also include dispensing a quantity of polarized gas so that the gas travels substantially axially out of the cavity of the solenoid to a dispensing port and/or positioning a plurality of cells in the cavity of the solenoid, each cell configured to be able to hold quantity of polarized gas (although selected ones may be empty or unused during certain operations).

Other embodiments are directed to hyperpolarizer systems for producing polarized gases. The systems include: (a) a plurality of cells, each configured to hold a quantity of target gas held therein, wherein at least one of the cells is an optical pumping cell configured to hold the target gas during spin-exchange polarization; (b) an optic system comprising a light source configured to generate circularly polarized light that is selectively transmitted to the at least one optical pumping cell during operation; (c) a magnetic field source positioned and configured to generate a magnetic holding field that covers the plurality of cells; (d) a controller configured to direct the operation of the optic system and sequence the polarization of the target gas in the cells; and (e) a polarization strength monitoring system in communication with each cell and the controller, the monitoring system configured to determine the polarization level of the target gas in each cell.

In operation, the controller can consider polarization level data provided by the monitoring system to selectively direct repolarization of previously polarized target gas (serial and/or concurrent spin-up of a plurality of cells) and sequences the order and timing that a batch of target gas from each cell is polarized and/or repolarized so that, at full operational status, the hyperpolarizer is adapted to hold a plurality of different batches of polarized target gas. At least two of the batches, and in certain embodiments, each batch, can have a different polarization decay cycle. The multiple batch configuration can increase the total amount of polarized gas that is available for dispensing from the hyperpolarizer.

The system can be configured to include a thermal source for elevating the temperature in the pumping cell during optical pumping. The thermal source can be the laser itself with the optical pumping cell held in an insulated cavity and/or at least one oven in thermal communication with the at least one optical pumping cell. The oven and/or thermal insulation cavity can be actively cooled (rather than letting the cell cool to room temperature naturally by turning the oven or laser off) post-polarization to facilitate cool-down of the polarized gas. The active cooling may be carried out by forcing cooled gas into the region of the hyperpolarizer proximate to (surrounding) the pumping cell.

Other embodiments are directed to mounting assemblies for a hyperpolarizer unit having multiple cells for polarizing target gas. The mounting assemblies include: a mounting plate and a plurality of cell bodies sized and configured to hold a quantity of target gas therein. The cell bodies are positioned on and/or in the mounting plate, and the cell bodies are formed of a material and/or coatings that inhibit the depolarization of polarized target gas held therein.

Additional embodiments are directed to computer program products for operating a hyperpolarizer having at least one optical pumping cell to produce polarized noble gas. The computer program product includes a computer readable storage medium having computer readable program code embodied in said medium. The computer-readable program code includes: (a) computer readable program code that determines the polarization level of each of a plurality of separate polarized gas batches held in individual cells in a hyperpolarizer over a desired time; (b) computer readable program code that selects the batch to be dispensed to a user upon request by a user based on the determined polarization levels of the batches of polarized gas held in the hyperpolarizer; and (c) computer readable program code that determines when and/or whether repolarization of respective batches of the polarized gas is desired based on the determined polarization levels.

Other embodiments are directed to apparatus for producing hyperpolarized gas. The apparatus includes: (a) a plurality of cells, each having a respective quantity of target gas held therein; (b) means for serially polarizing the target gas in and/or from the cells in a desired order to provide separate batches of polarized gas so that the polarized target gas in at least one of the cells has a different polarization decay cycle relative to the polarized target gas in the other cells during a monitoring period; (c) means for monitoring the polarization level of each of the batches of polarized target gas in the cells during a monitoring period; and (d) means for repolarizing the previously polarized target gas held in at least one of the cells when the polarization level falls below a predetermined value.

Advantageously, the present invention can provide increased timely production of hyperpolarized gas where cells can hold individual patient-sized quantities (such as 0.5-2 liters) of polarized gas that can be produced on-demand and dispensed in desired doses to support to a clinic or hospital.

All or selected operations, functions and/or configurations of the embodiments described above with may be carried out as methods, systems, computer program products, assemblies and/or devices as contemplated by the present invention.

The foregoing and other objects and aspects of the present invention are explained in detail herein.

### BRIEF DESCRIPTION OF THE DRAWINGS

FIGS. 1A-1F are schematic illustrations of polarization systems using multiple pumping and/or storage cells to provide multiple separately polarizable amounts of target gas according to embodiments of the present invention.

FIGS. 2A-2E are schematic illustrations of the serial production of polarized gas using a single optical pumping cell and multiple storage cells according to embodiments of the present invention.

FIG. 3A is a graph of the polarization level of serially produced batches of polarized gas in a polarization cell shown over time.

FIGS. 3B-3E are graphs of the polarization level of respective batches of polarized gas over time in individual holding cells in a polarizer system according to embodiments of the present invention.

FIG. 3F is a graph of the highest polarization level of a polarized gas that is available to be dispensed from the polarizer system over time, the highest polarization level being represented by a different batch identifier at different points in time according to embodiments of the present invention.

FIG. 3G is a graph of a polarization decay cycle (polarization level decreasing over time) as may occur in at least one holding cell after polarization.

FIG. 3H is a graph of an increase in polarization level over time that can occur in at least one optical pumping cell.

FIG. 4A is a schematic illustration of components of a polarizer configured for concurrent optical pumping of a plurality of optical pumping cells with respective amounts of target gas therein according to embodiments of the present invention.

FIGS. 4B and 4C are schematic illustrations that show serial optical pumping of selected optical pumping cells according to embodiments of the present invention.

FIG. 5 is a front partial cutaway view of a portion of a polarizer system according to embodiments of the present invention.

FIG. 6A is an exploded side view of a portion of a polarizer with a primary optical pumping cell according to embodiments of the present invention.

FIG. 6B is an exploded opposing side view of the device shown in FIG. 6A.

FIG. 6C is a front view of the assembled device shown in FIGS. 6A, 6B, 6D and 6E.

FIG. 6D is an exploded view of the oven assembly shown in FIG. 6C.

FIG. 6E is a top perspective view of the assembled device shown in FIG. 6C.

FIG. 6F is a schematic illustration of an insulated cavity that can use the laser beam to provide at least a portion of the heat used to elevate the optical pumping cell and that uses a cooling source that can cool the insulated cavity to maintain the insulated cavity, and hence the gas, at desired controlled temperatures.

FIG. 7A is a front view of a multi-holding cell configuration according to embodiments of the present invention.

FIG. 7B is a top view of the configuration shown in FIG. 7A.

FIG. 7C is a top view of the device shown in FIG. 7B with the top plate removed.

FIG. 8A is a top view of an alternate embodiment of a multi-holding cell configuration according to embodiments of the present invention.

FIG. 8B is a top view of a multi-holding cell arrangement similar to that shown in FIG. 7C but shown without certain structural and/or operative components according to embodiments of the present invention.

FIG. 9A is an enlarged partially exploded view of a single holding cell on a mounting plate suitable for a multi-cell configuration, such as those shown in FIG. 7C or 8B, according to embodiments of the present invention.

FIG. 9B is an enlarged side perspective view of the holding cell shown in FIG. 9A with an exploded view of an RF coil arrangement according to embodiments of the present invention.

FIG. 10A is a side perspective view of a mounting plate for holding a plurality of holding cells side by side according to embodiments of the present invention.

FIG. 10B is a side perspective view of a portion of a stacked mounting plate configuration that can be used to hold a cell with a target gas therein according to embodiments of the present invention.

FIG. 10C is an exploded view of multiple holding cells assembled together using the mounting plate illustrated in FIG. 10A according to embodiments of the present invention.

FIG. 11A is a top perspective view of a multi-cell arrangement according to embodiments of the present invention.

FIG. 11B is an enlarged front view of an alignment protuberance used to position the cells shown in FIG. 11A according to embodiments of the present invention.

FIG. 11C is a partially exploded view of the device shown in FIG. 11A with a ceiling plate positioned above the cells according to embodiments of the present invention.

FIG. 11D is a top view of the configuration shown in FIG. 11A.

FIG. 11E is a top perspective view of the configuration shown in FIG. 11C cover plate in place.

FIG. 11F is a partial cutaway side view of the configuration shown in FIG. 11E.

FIG. 11G is an enlarged exploded view of an alternate alignment protuberance assembly according to embodiments of the present invention.

FIG. 11H is a side perspective view of the assembly shown in FIG. 11G in position.

FIG. 11I is an enlarged bottom perspective view of a cell held on a mounting plate using the protuberance assembly shown in FIG. 11H.

FIG. 12 is a top perspective view of yet another multi-cell mounting arrangement according to embodiments of the present invention.

FIG. 13A is a top view of a cell used to hold target (polarized and/or unpolarized) gas according to embodiments of the present invention.

FIG. 13B is a front view of the cell shown in FIG. 13A.

FIG. 13C is a front view of an alternate cell configuration according to embodiments of the present invention.

FIG. 13D is a front view of yet another cell configuration according to embodiments of the present invention.

FIG. 14A is a front, exploded, partial cutaway view of an optical pumping and oven member according to embodiments of the present invention.

FIG. 14B is an enlarged top view of a portion of the oven member and cell shown in FIG. 14A.

FIG. 14C is an enlarged side view of the optical pumping cell shown in FIG. 14B.

FIG. 15A is an exploded side view of a heating element according to embodiments of the present invention.

FIG. 15B is an exploded top view of the heating element shown in FIG. 15A with side oven housing walls.

FIG. 15C is an exploded top view of the heating element and oven walls shown in FIG. 15B with an optical cell positioned therein.

FIG. 15D is a side view of the device shown in FIG. 15C.

FIG. 15E is a front view illustrating the device shown in FIG. 15D assembled.

FIG. 16A is a schematic illustration of a gas transfer arrangement that can be used to transfer gas between (to and/or from) an optical pumping cell and a storage cell using a resilient member and pressure chamber according to embodiments of the present invention.

FIG. 16B is a schematic illustration of a gas transfer arrangement that can be used to transfer gas between (to and/or from) an optical pumping cell and a storage cell using a pressure chamber according to embodiments of the present invention.

FIG. 16C is a schematic illustration of an alternate configuration of a resilient member according to embodiments of the present invention.

FIG. 16D is a side view of the resilient member illustrated in FIG. 16C.

FIG. 17A is an exploded view of one arrangement that can be used to implement the transfer arrangement shown in FIG. 16A according to embodiments of the present invention.

FIG. 17B is a bottom perspective view illustrating the device shown in FIG. 17A.

FIG. 17C is a side, perspective, partial cutaway view of the assembled device shown in FIG. 17B.

FIG. 17D is an exploded top perspective view of the device shown in FIG. 17A.

FIG. 17E is a side partially exploded view of an example of a polarizer assembly using the device shown in FIGS. 17A-17D according to embodiments of the present invention.

FIG. 17F is a front view of the device shown in FIG. 17E with a magnetic field generator shown partially cut away according to embodiments of the present invention.

FIG. 18A is a top perspective view of an optic system aligned over the optical pumping cell in the magnetic field generator according to embodiments of the present invention.

FIG. 18B is a side view of the device shown in FIG. 18A.

FIG. 18C is a front partial cutaway view of a polarizer system with the device shown in FIG. 18A in a cabinet according to embodiments of the present invention.

FIG. 18D is a partial cut away front view of a polarizer system in a cabinet according to other embodiments of the present invention.

FIG. 19A is a partial cut away front view of a polarizer system in a cabinet with a diagonally oriented magnetic field generator and other aligned components according to embodiments of the present invention.

FIG. 19B is a partial cut away side view of the system shown in FIG. 19A.

FIGS. 20A-20D are top perspective views of a movable optic system according to embodiments of the present invention, as the optic system translates to align with different underlying cells in different locations.

FIG. 21 is a partial cut away front view of a polarizer system with a substantially horizontally oriented magnetic field generator according to alternate embodiments of the present invention.

FIG. 22A is a side view of a sealed cell used to hold target gas according to embodiments of the present invention.

FIG. 22B is an enlarged front view of the lower stem portion of the cell shown in FIG. 22A.

FIG. 22C is a side perspective view of a valve assembly suitable for use in controlling gas transfer from cells according to embodiments of the present invention.

FIG. 22D is a side view of the cell shown in FIG. 22A in the assembly shown in FIG. 22C illustrating an enclosed seal release mechanism according to embodiments of the present invention.

FIG. 23 is a block diagram of operations that can be used to carry out embodiments of the present invention.

FIG. 24 is a block diagram of operations that can be used to carry out embodiments of the present invention.

FIG. 25 is a block diagram of operations that can be used to carry out embodiments of the present invention.

FIG. 26 is a schematic illustration of a system suitable for operating a polarizer system according to embodiments of the present invention.

### DETAILED DESCRIPTION OF EMBODIMENTS OF THE INVENTION

The present invention will now be described more fully hereinafter with reference to the accompanying figures, in which preferred embodiments of the invention are shown. This invention may, however, be embodied in many different forms and should not be construed as limited to the embodiments set forth herein. Like numbers refer to like elements throughout. In the drawings, layers, regions, or components may be exaggerated for clarity. In the figures, broken lines indicate optional features unless described otherwise.

In the description of the present invention that follows, certain terms may be employed to refer to the positional relationship of certain structures relative to other structures. As used herein the term “forward” and derivatives thereof refer to the general direction the target gas or target gas mixture travels as it moves through the hyperpolarizer system; this term is meant to be synonymous with the term “downstream,” which is often used in manufacturing environments to indicate that certain material being acted upon is farther along in the manufacturing process than other material. Conversely, the terms “rearward” and “upstream” and derivatives thereof refer to the directions opposite, respectively, the forward and downstream directions.

Also, as described herein, polarized gases are produced and collected and may, in particular embodiments, be frozen, thawed, be used alone and/or combined with other constituents, for MRI and/or NMR spectroscopy applications. For ease of description, the term “frozen polarized gas” means that the polarized gas has been frozen into a solid state. The term “liquid polarized gas” means that the polarized gas has been or is being liquefied into a liquid state. Thus, although each term includes the word “gas,” this word is used to name and descriptively track the gas that is produced via a hyperpolarizer to obtain a polarized “gas” product. Thus, as used herein, the term “gas” or “target gas” has been used in certain places to descriptively indicate a hyperpolarized noble gas product and may be used with modifiers such as “solid”, “frozen”, and “liquid” to describe the state or phase of that product. As also used herein, the term “polarized gas”, “target gas” and/or “polarized target gas” includes at least one intended target gas of interest (such as, but not limited to, ³He and/or ¹²⁹Xe) and may include one or more other constituents such as other carrier or blending gases, buffer gases, or carrier liquids as desired. Further, the terms “polarize”, “polarizer”, “polarized”, and the like are used interchangeably with the terms “hyperpolarize”, “hyperpolarizer”, “hyperpolarized” and the like.

Various techniques have been employed to accumulate and capture polarized gases. For example, U.S. Pat. No. 5,642,625 to Cates et al. describes a high volume hyperpolarizer for spin-exchange polarized noble gas and U.S. Pat. No. 5,809,801 to Cates et al. describes a cryogenic accumulator for spin-polarized ¹²⁹Xe. As used herein, the terms “hyperpolarize,” “polarize,” and the like, are used interchangeably and mean to artificially enhance the polarization of certain noble gas nuclei over the natural or equilibrium levels. Such an increase is desirable because it allows stronger imaging signals corresponding to better MRI images of the substance and a targeted area of the body. As is known by those of skill in the art, hyperpolarization can be induced by spin-exchange with an optically pumped alkali-metal vapor or alternatively by metastability exchange. See Albert et al., U.S. Pat. No. 5,545,396.

Generally described, hyperpolarizer systems include optic systems with a laser source, such as a diode laser array, and optic beam forming or focusing components such as beam splitters, lenses, mirrors or reflectors, wave plates or retarders, and/or other optical components for providing the circularly polarized light source to the target gas held in an optical pumping cell. For ease of description, the term “optic system” as used herein includes the optical pumping components used to generate and/or manipulate the circularly polarized light.

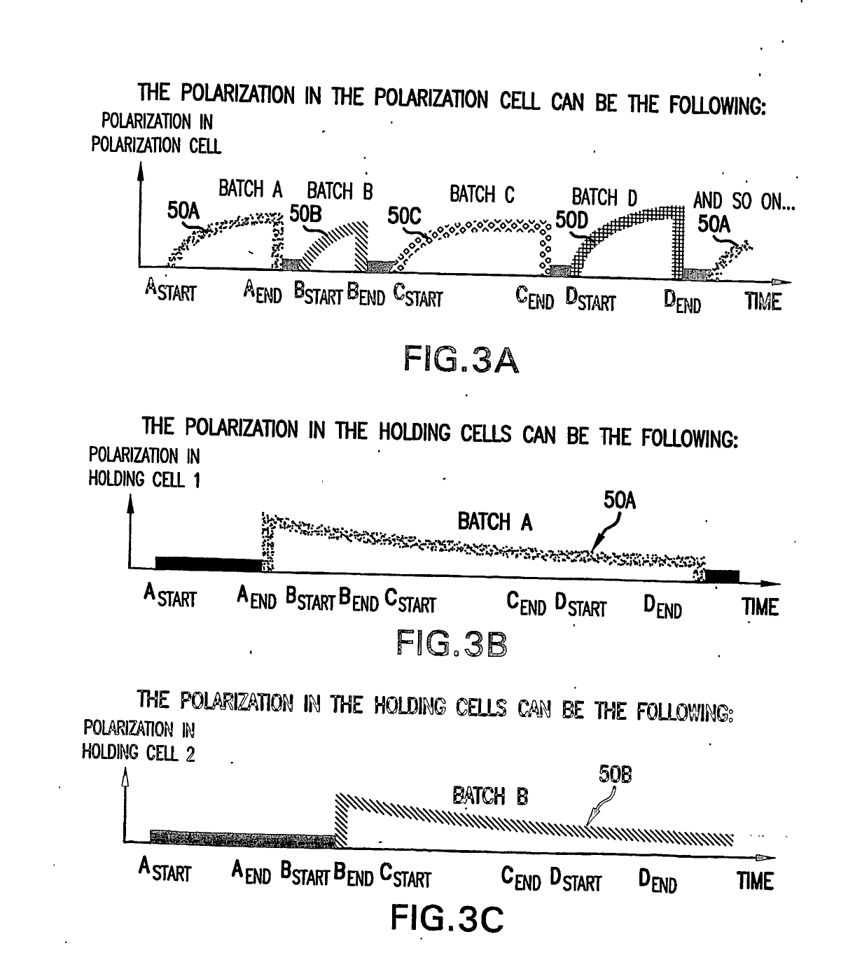

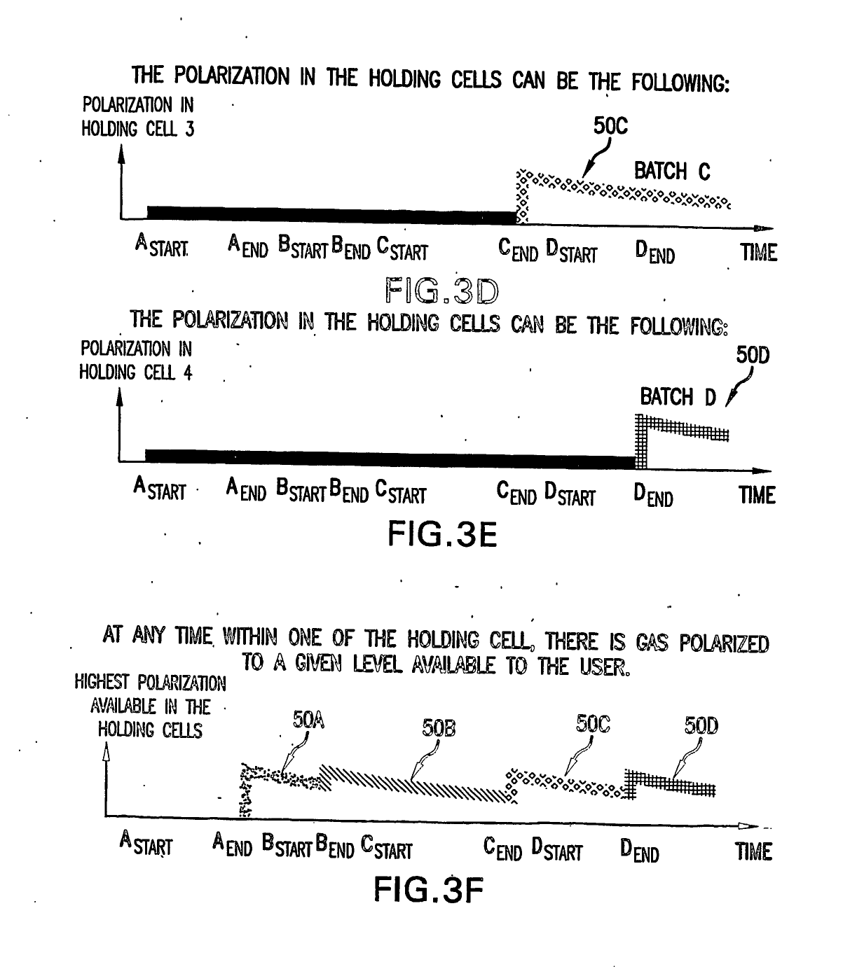

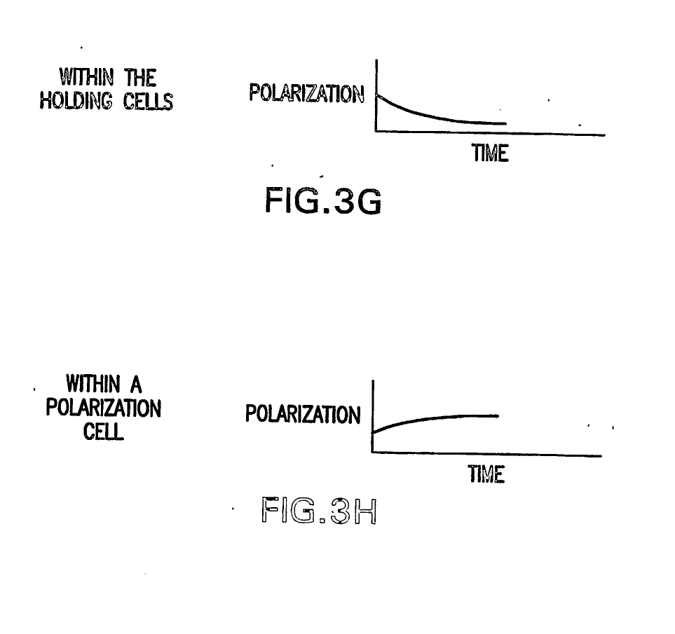

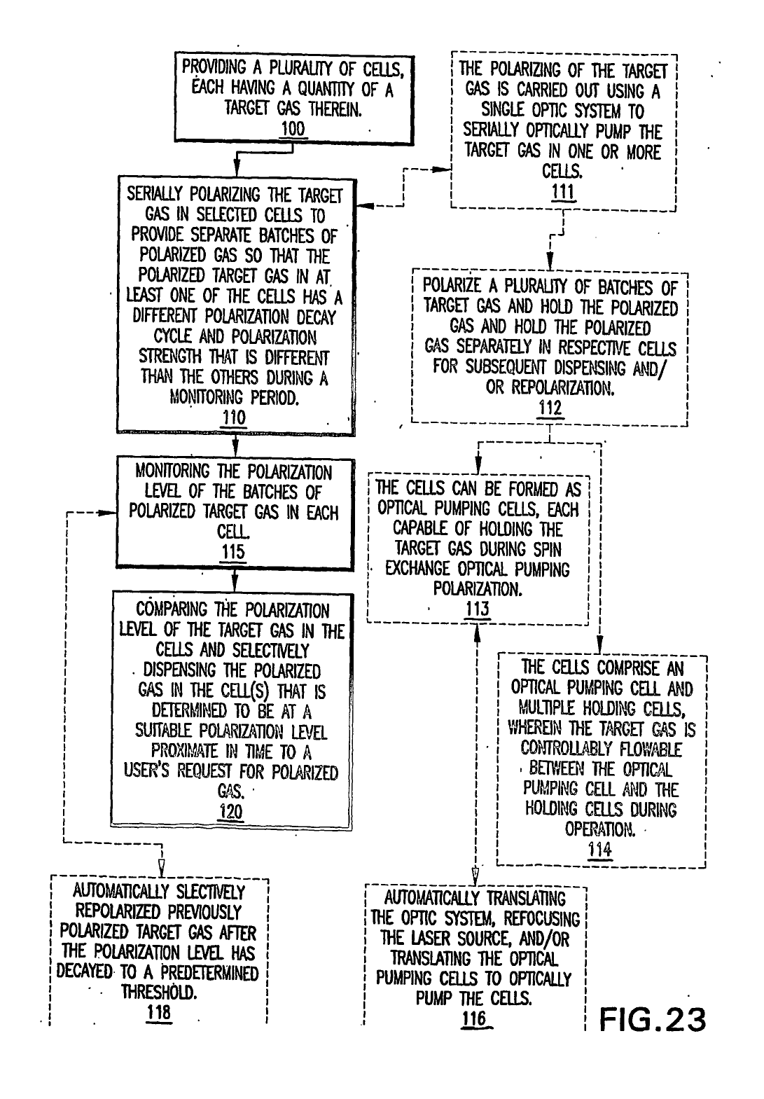

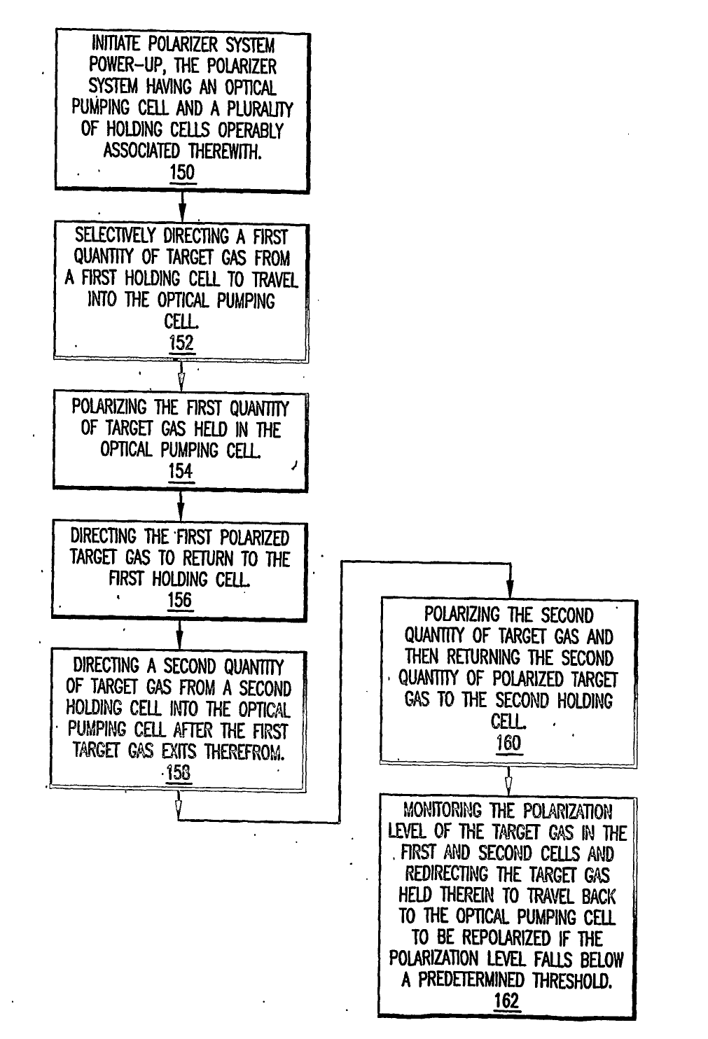

FIG. 23 illustrates an example of operations that can be carried out according to embodiments of the present invention. As shown, a plurality of cells, each being configured to hold a quantity of target gas, is provided (block 100). Block 100 is also contemplated to mean that a quantity of a target gas is provided to a plurality of cells. It is noted that certain of the cells may remain empty or unused during operation. The target gas in desired cells (typically each cell) can be polarized to provide separate batches of polarized gas. The polarized target gas in at least one of the cells can have a different polarization strength and/or polarization decay cycle relative to the polarized target gas in another holding cell or optical pumping cell. FIG. 3G illustrates the polarization decay cycle (polarization level decreasing over time) that can occur in at least one holding cell after polarization while FIG. 3H is a graph of an increase in polarization level over time that can occur in at least one optical pumping cell.

The polarization strength at any given time for a given batch may be periodically monitored during a monitoring period (block 110). In certain embodiments, the polarization level of the individual batches of polarized gas (held in respective cells) can be monitored (block 115). The polarization level of the gas in the cells can be compared and the hyperpolarizer can be configured to selectively dispense the polarized gas held in the cell(s) that is determined to be at a suitable polarization level (block 120). The comparison and/or selective dispensing can be carried out dynamically proximate in time to a planned dispense operation and/or upon request by a user or clinician for a dispensed amount of polarized gas. Selected ones of the batches of polarized gas can be repolarized automatically after the polarization level has decayed below a desired level (block 118), typically below a predetermined threshold level. The “acceptable” level may be adjustable by the system dependent upon the requested dispense amount or planned use (NMR or MRI) to provide the desired formulation.

In certain embodiments, the polarizing of the target gas can be carried out using single optic system to serially optically pump the target gas in one cell or concurrently pump selected ones of the gas in one or more of the cells (meaning physically held in one or more of the cells before or during polarization) (block 111). As such, a plurality of batches of polarized gas can be serially polarized and held in respective cells for subsequent dispensing and/or repolarization (block 112). The cells can be formed as optical pumping cells, each capable of holding the target gas during polarization using spin-exchange optical pumping (block 113). The optic system can be automatically translated or refocused and/or the optical pumping cells can be translated to align the optic system with the selected one or ones of the optical pumping cells during polarization (block 116). In other embodiments, the cells can comprise an optical pumping cell and a plurality of holding cells in fluid communication therewith. The target gas can be flow controlled to travel between the optical pumping cell and the holding cells during operation (block 114).

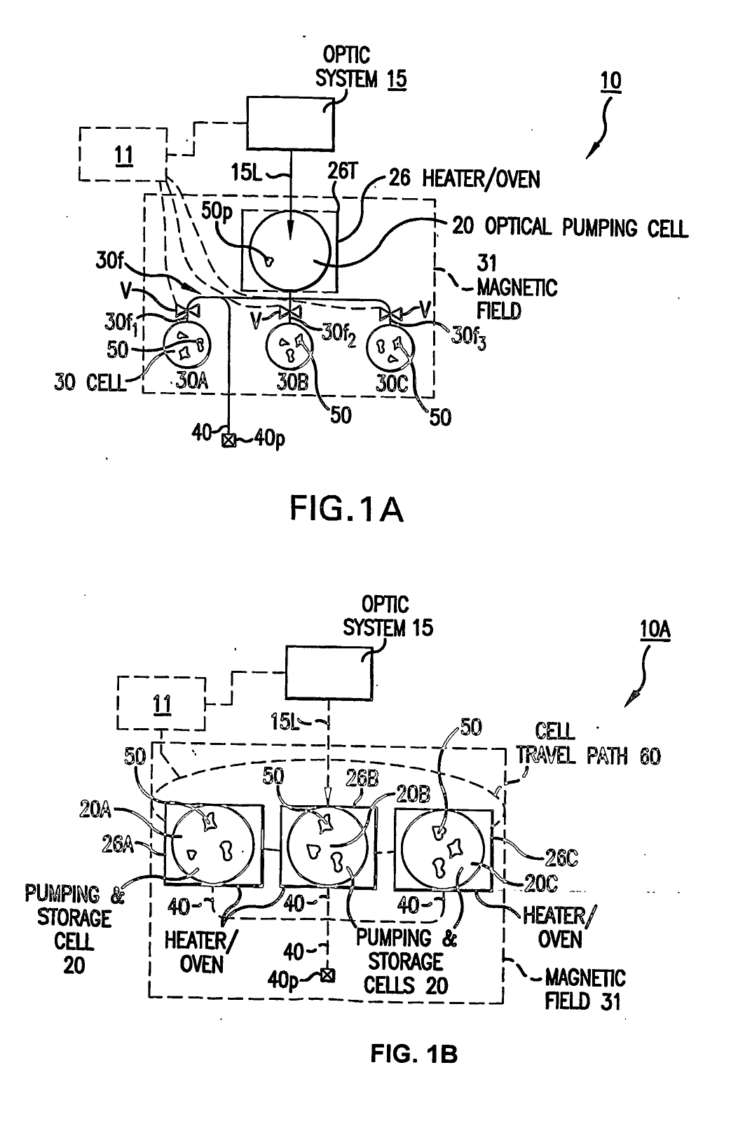

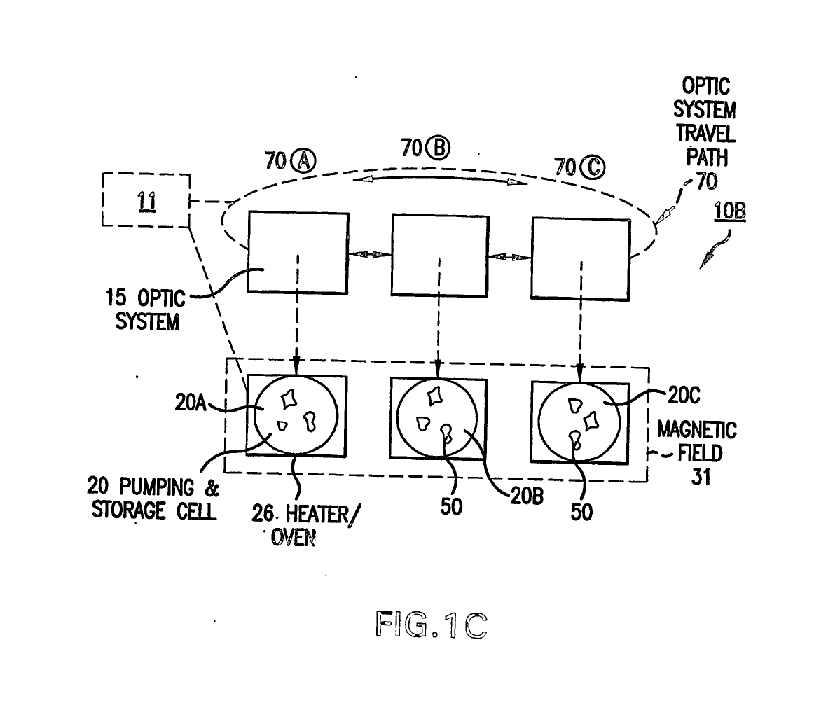

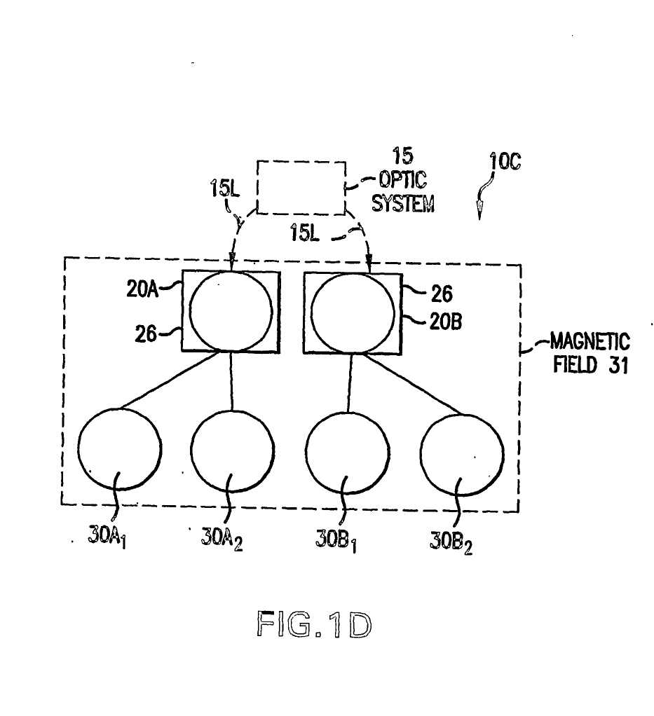

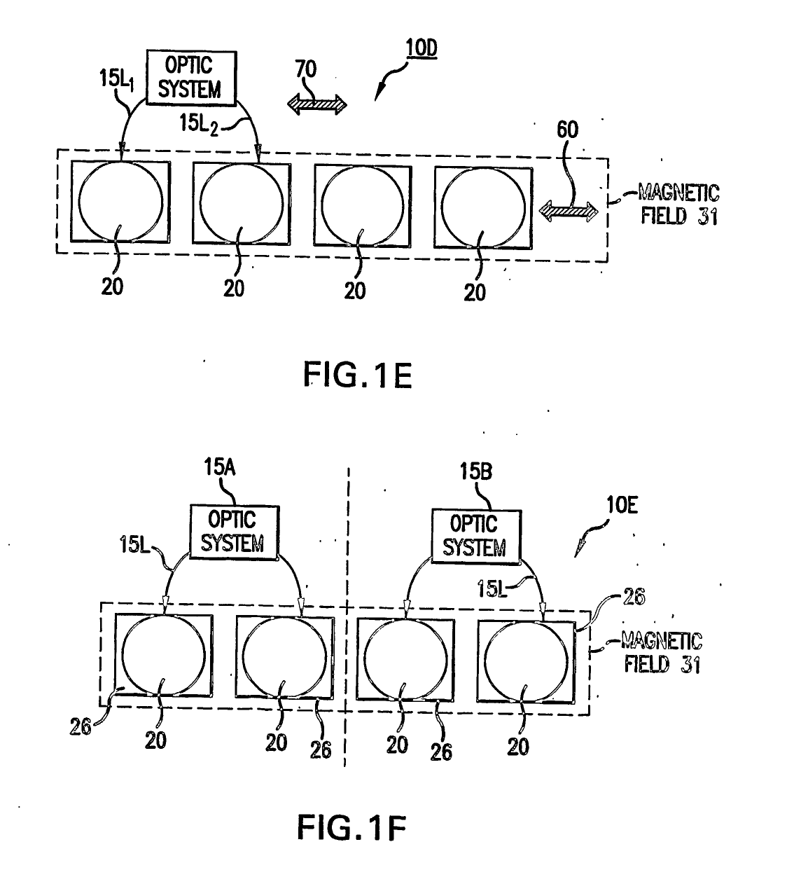

Turning now to FIG. 1A, one example of a hyperpolarizer system 10 is shown. In this embodiment, the system 10 includes an optic system 15 that generates and transmits the polarized light 15L, an optical pumping cell 20, a thermal source 26T, shown as an oven 26 that is configured to provide heat to the optical pumping cell 20 during the polarizing operation, a magnetic field source 31, and a plurality of holding cells 30 (shown for illustration purposes as three cells 30A, 30B, and 30C). Other numbers of cells 30 (such as two, four, five, six, or more) can also be used in embodiments of the present invention.

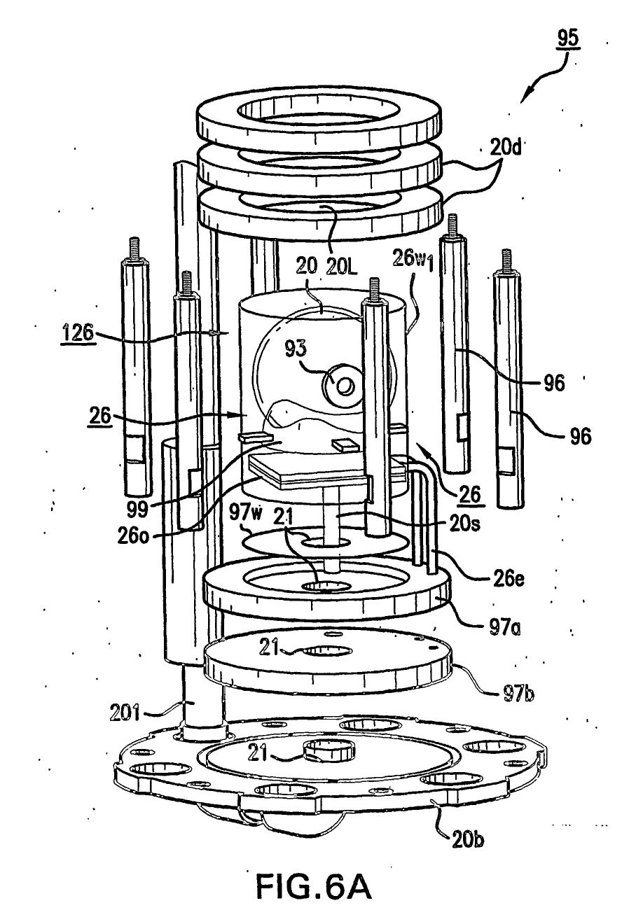

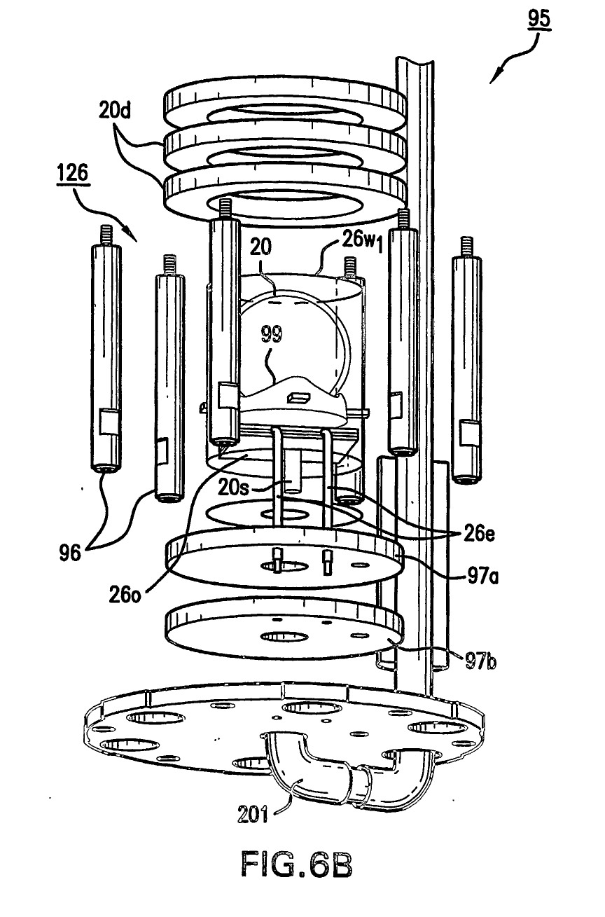

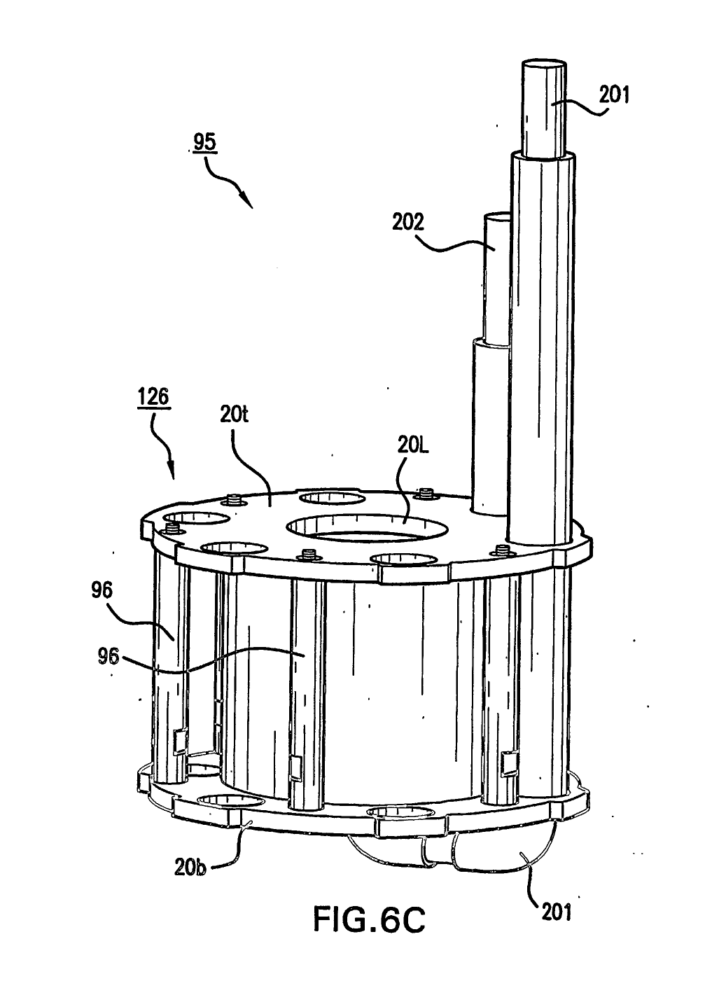

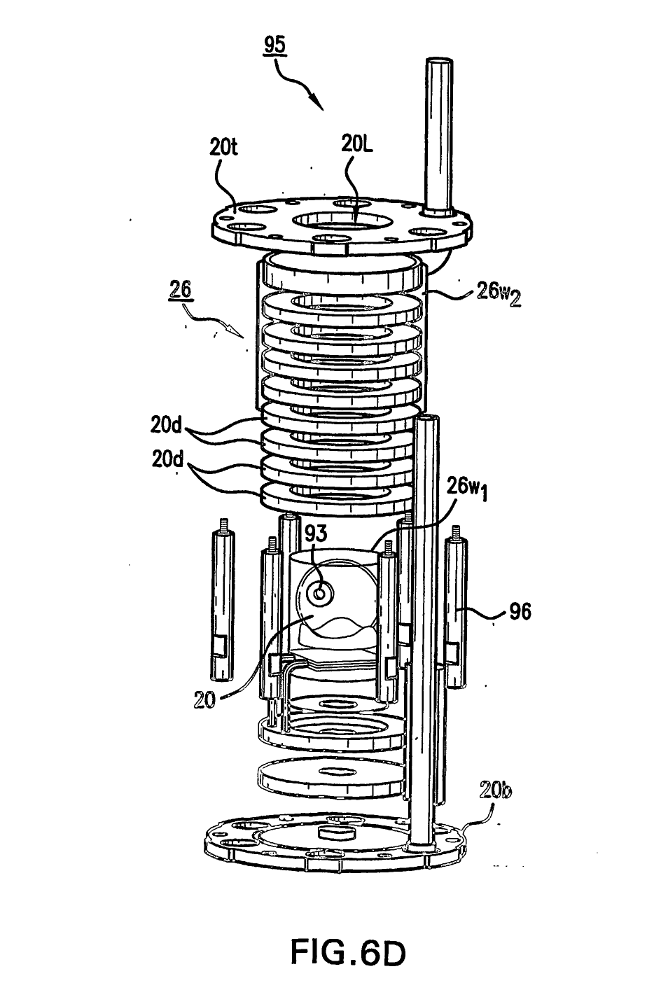

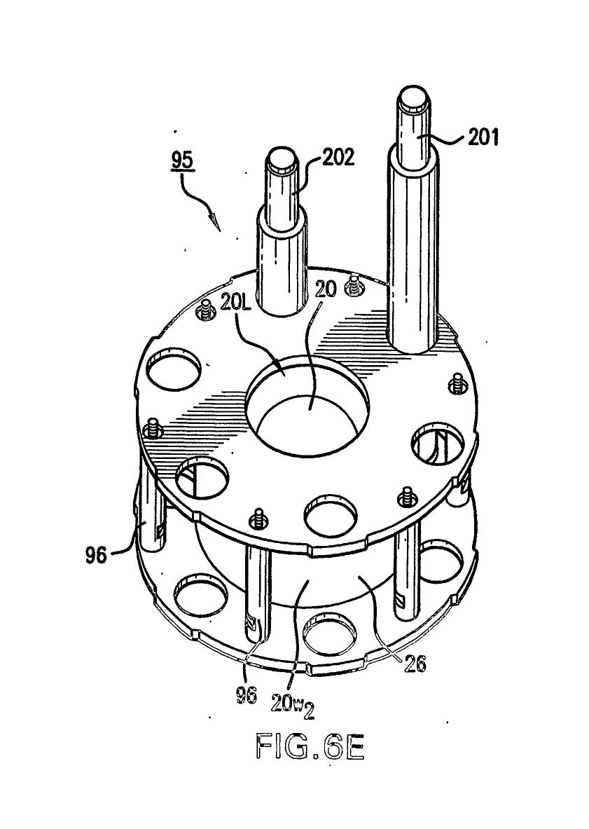

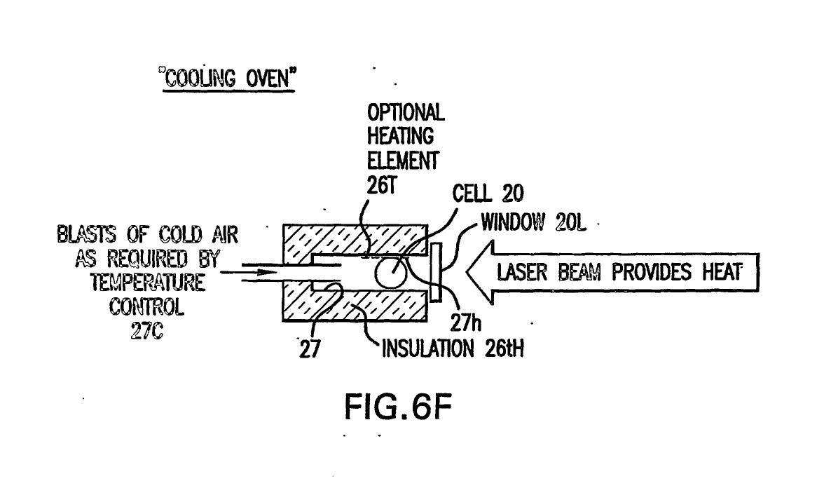

The thermal source 26T may be any suitable thermal configuration that can provide heat to elevate the temperature of the target gas in the optical pumping cell(s) 20 during spin-up (optical pumping). As will be discussed further below, one example of a thermal source 26 includes, as schematically shown in FIG. 1A, a conventional oven with a heating element that encases the optical cell 20 and has a window that allows the laser beam to pass through. In other embodiments, as shown in FIG. 6F, a thermally insulated cavity 27 is configured to hold the cell 20 can have an active cooling source 27c that is configured to use the laser beam to heat the gas. A window 20L can close the cavity 27 over the cell 20 and allow the laser beam to enter therein. The active cooling source 27c is in fluid communication with the cavity 27 and is configured to introduce a coolant fluid (typically blasts of cold air) into the cavity at desired times to cool and/or maintain the temperature of the target gas and/or cell 20 at a desired temperature. Thus, the thermal insulation 26ₜₕ is configured with a sufficient thickness, material, or combinations of both to provide sufficient thermal insulating ability to be able to capture the laser energy and elevate the temperature of the target gas in the cell 20 during optical pumping. Optionally, the thermal cavity 27 can include a heating element 27h that can be used to facilitate the initial heating process (then switching to an intermittent cooling to maintain the temperature at stead state using the laser alone). Thermal source embodiments are discussed further below with reference to FIGS. 6A-6F.

Referring back to FIG. 1A, the system 10 can include a single optic system 15 (with a single laser source) that can be used to serially polarize target gas released from the holding cells 30 to the optical pumping cell. In other embodiments, the system 10 can include a plurality of optic systems 15, one for each optical pumping cell or each desired number of optical pumping cells 20. The magnetic field 31 can be configured with a size and sufficient homogeneity to extend to cover both the optical pumping cell 20 and the holding cells 30.

The hyperpolarizer 10 is configured with individually selectable enclosed gas flow travel paths 30f₁, 30f₂, 30f₃ (the gas flow path being referred to generally as 30f) that extend between the respective cells 30 and the optical pumping cell 20. The gas flow paths 30f₁, 30f₂, 30f₃ can be operably associated with one or more (automated) valves (identified with the letter “V”) in the travel paths to control the release and flow direction of the gas in the system.

In operation, a batch amount of target gas 50 is released from the cell 30 and directed to the optical pumping cell 20 where it is polarized. After polarization, the polarized target gas 50p can be returned to its respective cell 30. The polarized target gas 50p then decays back to a non-polarized level. This sequence of operations can be repeated until all of the holding cells 30 hold polarized gas 50p. As each batch is polarized at a different time, each will have different decay profiles (strength versus time) with a different polarization strength at any given point in time. Once a batch has decayed to a threshold value, it can be redirected to the pumping cell 20 to be repolarized.

The release and transfer of the gas can be automated and controlled by a controller 11. The controller 11 may also include computer program code with instructions that control the sequencing of operations and/or the activation of the optic system 15. The hyperpolarizer 10 may also include a dispense flow path 40 and associated dispense port 40p that can allow the polarized gas 50p to be dispensed. The controller may be configured to automatically monitor the polarization level of the individual batches of polarized gas 50p that are held in respective cells 30 to selectively dispense the polarized gas 50p that has a desired polarization level proximate in time to a planned and/or requested dispensing output.

FIG. 24 illustrates exemplary operations of a hyperpolarizer having multiple holding cells and a respective optical pumping cell. As shown, power-up of the polarizer system can be initiated (block 150). A first quantity of target gas from a first holding cell can be selectively directed to travel into the optical pumping cell (block 152). The first quantity of target gas can be polarized in the optical pumping cell (block 154). The first polarized target gas can be returned to a selected cell, such as the first holding cell or a different holding cell (block 156). A second quantity of target gas from a second holding cell can be directed into the optical pumping cell after the first polarized gas exits therefrom (block 158). The second quantity of target gas can be polarized and then returned to a holding cell, such as the second holding cell (block 160). The polarization level of the (polarized) target gas in the selected cells, such as the first and second holding cells, can be monitored and the target gas held therein can be redirected to travel back into the optical pumping cell to be repolarized if the polarization level falls below a predetermined or desired threshold (block 162). Thus, the polarized gas may be returned to its original holding cell or another holding cell. Target gas may also be transferred to desired cells from a source of unpolarized gas to replace polarized gas that has been dispensed from the system.

In other embodiments, the first polarized gas can be transferred to a different holding cell or a designated polarized holding cell or cells that are different from the first non-polarized holding cell. In addition, gas from more than one holding cell may be directed to flow into the optical pumping cell during a single polarization procedure to produce increased quantity batches.

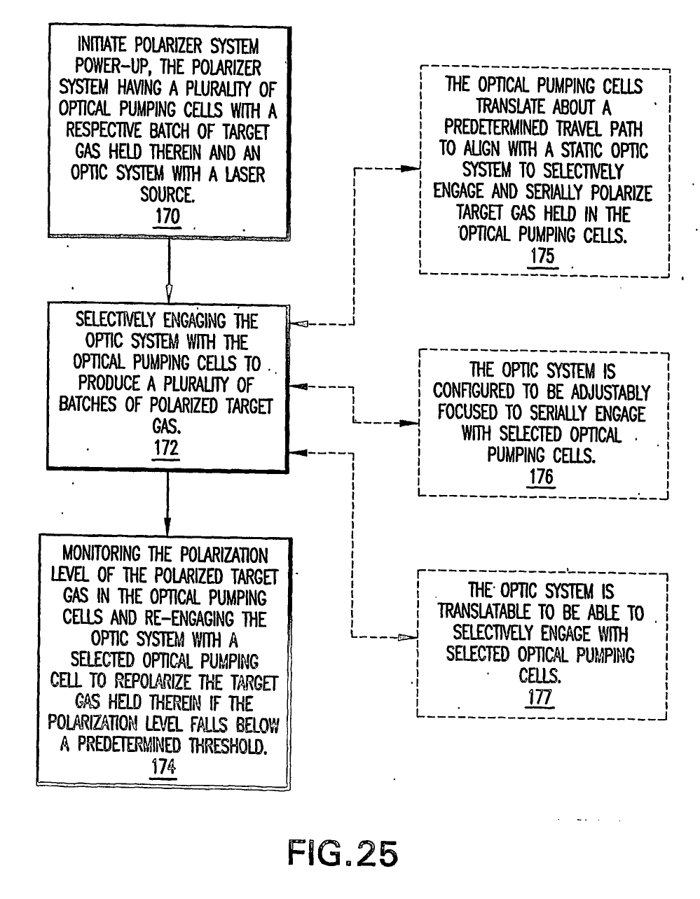

FIG. 25 shows operations that can be carried out using multiple optical pumping cells according to alternative embodiments of the present invention. As shown, initiation of the power-up of the polarization system having a central laser source and a plurality of optical pumping cells with respective batches of target gas held therein can be commenced (block 170). The optic system can be selectively engaged with one or more optical pumping cells to produce a plurality of batches of polarized gas (block 172). The polarization can be carried out to serially polarize selected target gas held in respective cells or to concurrently polarize target gas held in two or more cells. The polarization level of the polarized target gas in the cells can be monitored and the optic system can be re-engaged with selected optical pumping cells to repolarize the target gas held therein if the polarization level falls below a desired (typically predetermined) threshold value or level (block 174).

The optical pumping cells may be configured to translate about a predetermined travel path to align with an optic system 15 to selectively engage and serially polarize target gas held in the optical pumping cells (block 175). Alternatively, or in addition, the optic system can be configured to alter its light transmission path to adjustably redirect the light to serially engage with one or selected ones of the optical pumping cells (block 176). In other embodiments, the optic system or portions thereof are translatable to selectively engage with one or more selected optical pumping cells (block 177).

Turning now to FIG. 1B, an alternative embodiment of a hyperpolarizer system 11A is shown. The system 11A includes an optic system 15 and a plurality of optical pumping cells 20 (illustrated as three cells, 20A, 20B, 20C). As for the holding cells above, greater or lesser numbers of the optical pumping cells can be employed, such as 3, 4, 5, 6, or more. Each optical pumping cell 20 can include its own oven 26 (shown as ovens 26A-26C). The optic system 15 may be statically configured with the optical pumping cells 20 configured to translate to place a selected one or more cells 20 in optical communication with the light generated from the optic system 15. As such, the optical pumping cells 20 can be mounted in the hyperpolarizer 10A so that each travels along a defined travel path 60. The travel path 60 may be an endless path or other desired configuration. As will be discussed further below, the cells 20 may be held on a mounting plate that translates to index and/or position the cells 20 in the desired location with respect to the optic system 15 for a polarizing operation. In particular embodiments, the mounting plate may be circular and the plate configured to automatically rotate in response to an automated track and/or drive system.

FIG. 1C illustrates a hyperpolarizer 10C with multiple optical pumping cells 20 (illustrated as three cells 20A, 20B, 20C) similar to that shown in FIG. 1B. In this embodiment, at least a portion of the optic system 15 is configured to translate in a desired travel path 70 to align the circularly polarized light with one or more selected optical pumping cells 20. As shown, the optic system 15 can serially translate a plurality of times (shown as positions 70A, 70B, 70C) to be located over a respective optical pumping cell during polarization of the target gas 50 held in the cell 20. The optic system travel path 70 may be defined by a predetermined track and drive system (using conventional translation mechanisms such as gears, linkages, chains, belts, conveyors, and the like) so as to automatically translate to a desired location upon command from the controller 11. The optic system travel path 70 may be an endless path, such as a substantially circular path or other desired shape.

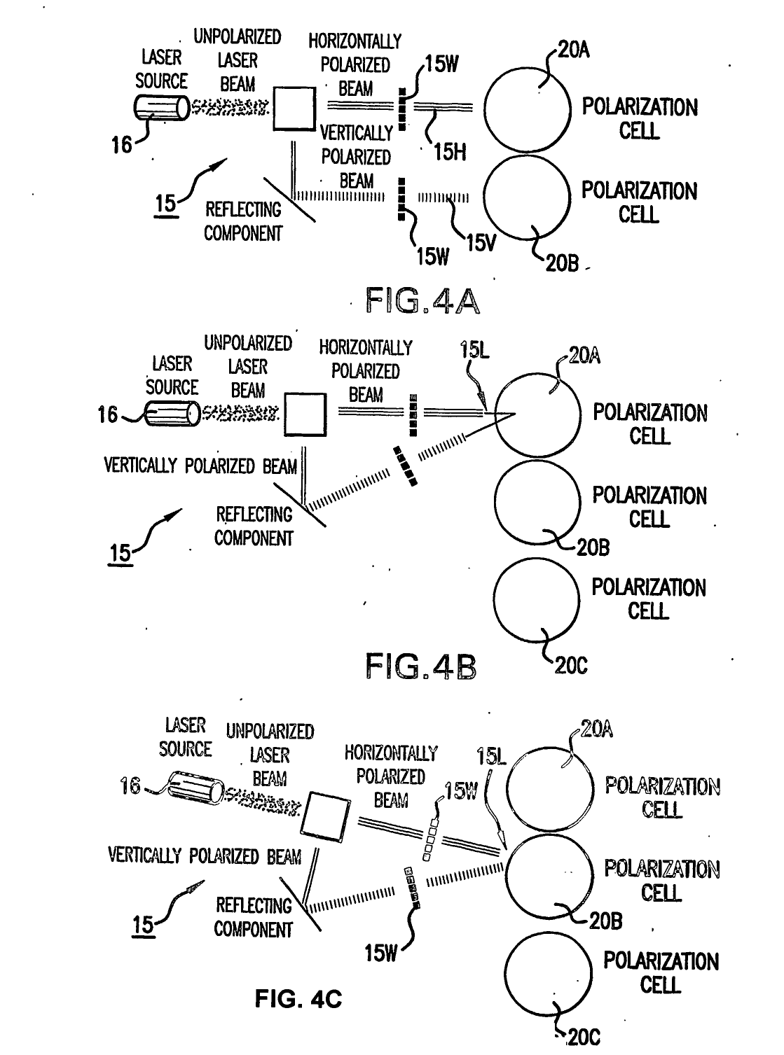

In other embodiments, the optic system 15 may be a common optic system 15 that is primarily static (stationary) but have refocusing components (mirrors, lenses and the like) that direct the light beam to travel to the desired location (s) to pump the target gas in the selected cell 20 (or cells) (as will be more fully discussed for FIGS. 4A-4C).

FIG. 1D illustrates a system similar to that shown in FIG. 1A, with a hyperpolarizer 10C that includes an optic system 15 and two optical pumping cells 20A, 20B, each with respective target gas feeder cells 30A₁, 30A₂ and 30B₁, 30B₂, respectively. The optic system 15 may be configured to concurrently or serially optically pump the cells 20A, 20B. The cells 20A, 20B may be static or dynamically mounted to align with the optic system 15. As before, the optic system 15 may also be translatable or have an adjustable light transmission path.

FIG. 1E illustrates that hyperpolarizers 10D using combinations of the above-described embodiments may also be employed. For example, as shown, the embodiments shown in FIGS. 1B and 1C can be combined so that the optic system 15 and the optical pumping cells 20 may each be configured to translate about respective travel paths 60, 70 during operation. FIG. 1F illustrates that the hyperpolarizer 10E can employ two (or more) optic systems 15A, 15B (each with its own laser source or sources), each can be operably associated with a respective optical pumping cell or cells (shown as two cells) and, optionally, feeder or holding cells (not shown).

The magnetic field 31 shown by the broken lines in FIGS. 1A-1F, which covers the optical pumping cell or cells 20 and the holding cells 30 (where used) can be provided by any suitable magnetic field source, such as permanent or electromagnets. Typically, a low magnetic field strength is used, typically about 500 Gauss or less, and more typically about 100 Gauss or less, with sufficient homogeneity to inhibit depolarizing influences during production and storage of the polarized target gas. In particular embodiments, a field strength of between 7-20 Gauss may be appropriate. In certain embodiments, a magnetic field homogeneity on the order of 10⁻³ cm⁻¹ (Gauss) is desirable, at least for the regions covering hyperpolarized gas for any length of time. Conventionally, Helmholtz coils have been used. The magnetic field 31 can also be configured to extend a distance sufficient to cover the gas dispensing path 40 and port 40p (FIGS. 1A and 1B). In particular embodiments, the field 31 may be further generated, formed or shaped to extend to cover the receiving containers of polarized gas during dispensing (not shown).

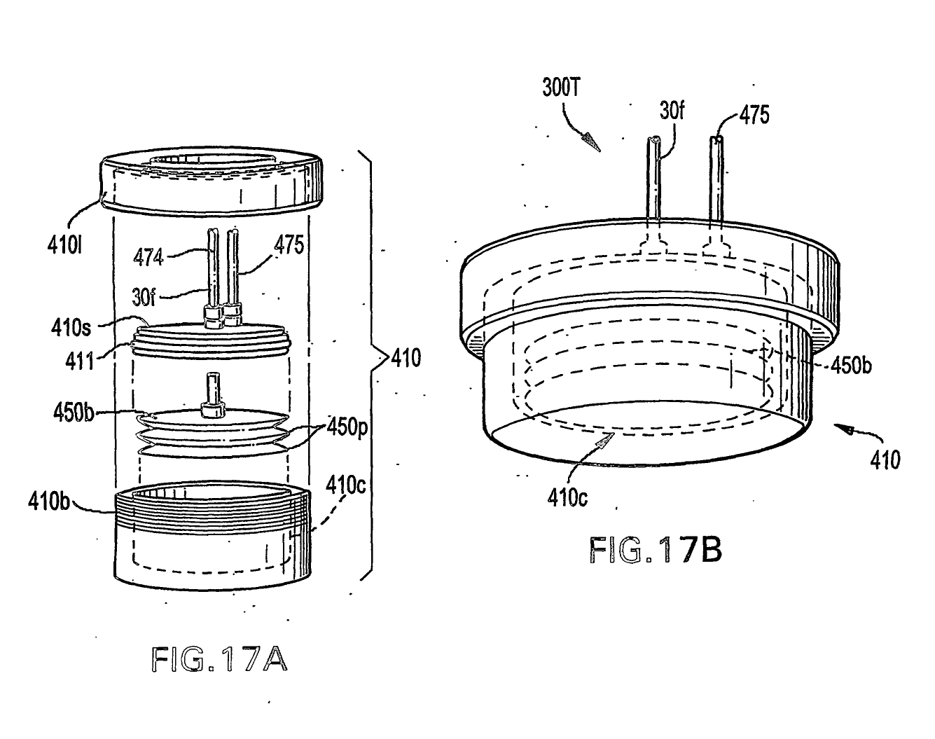

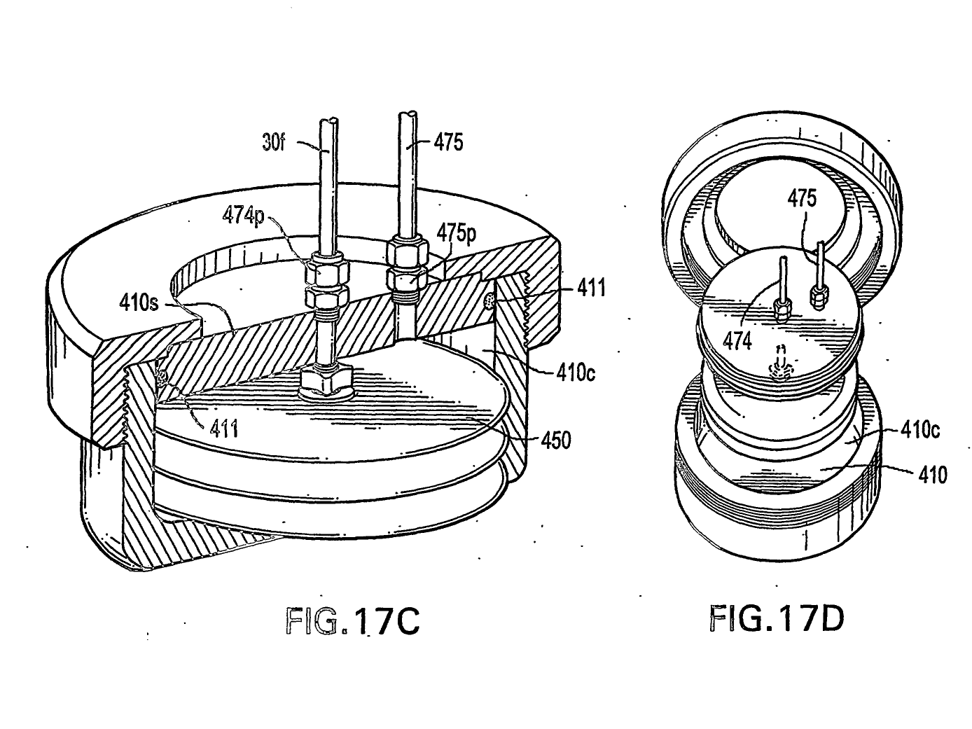

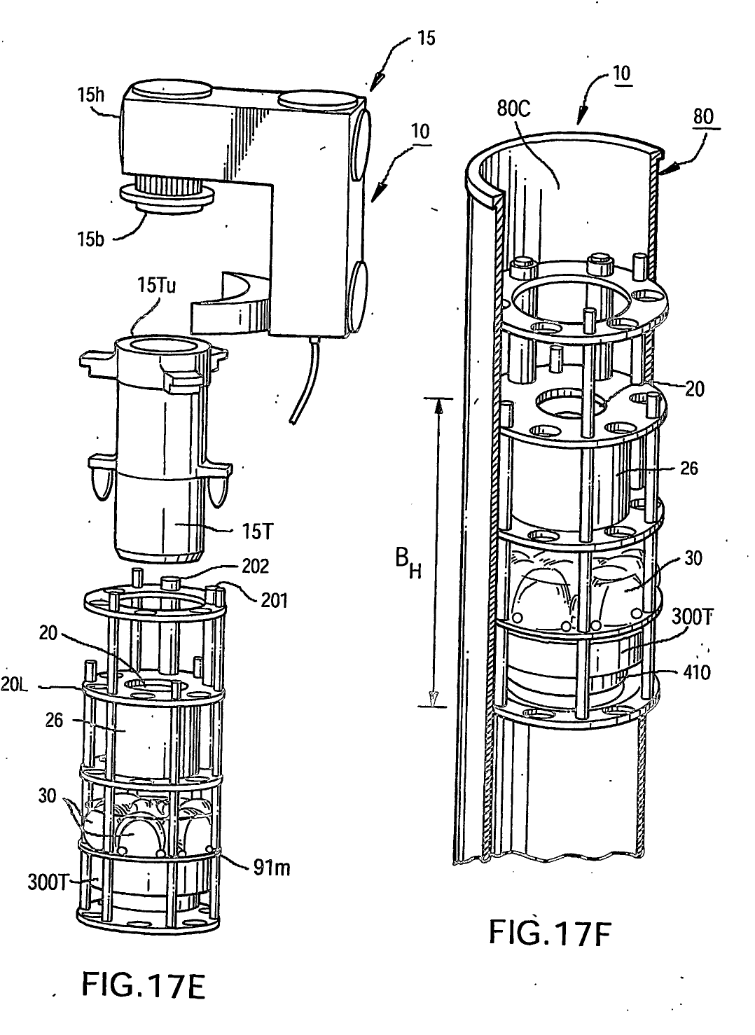

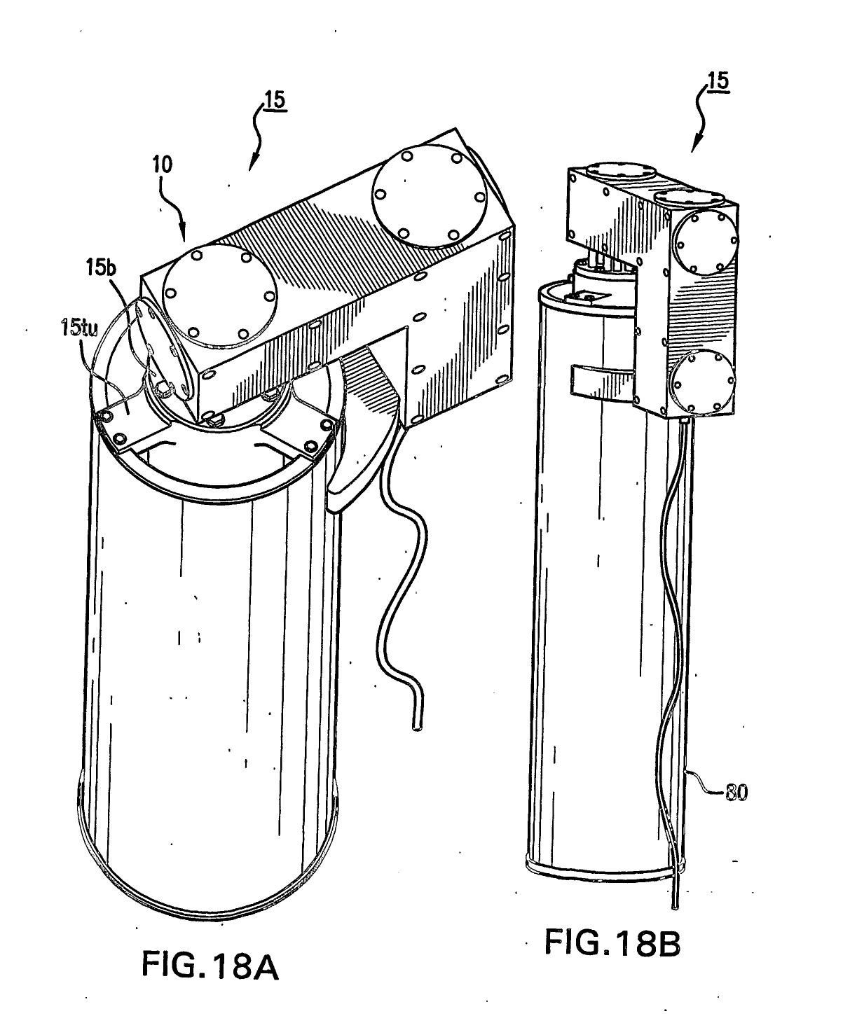

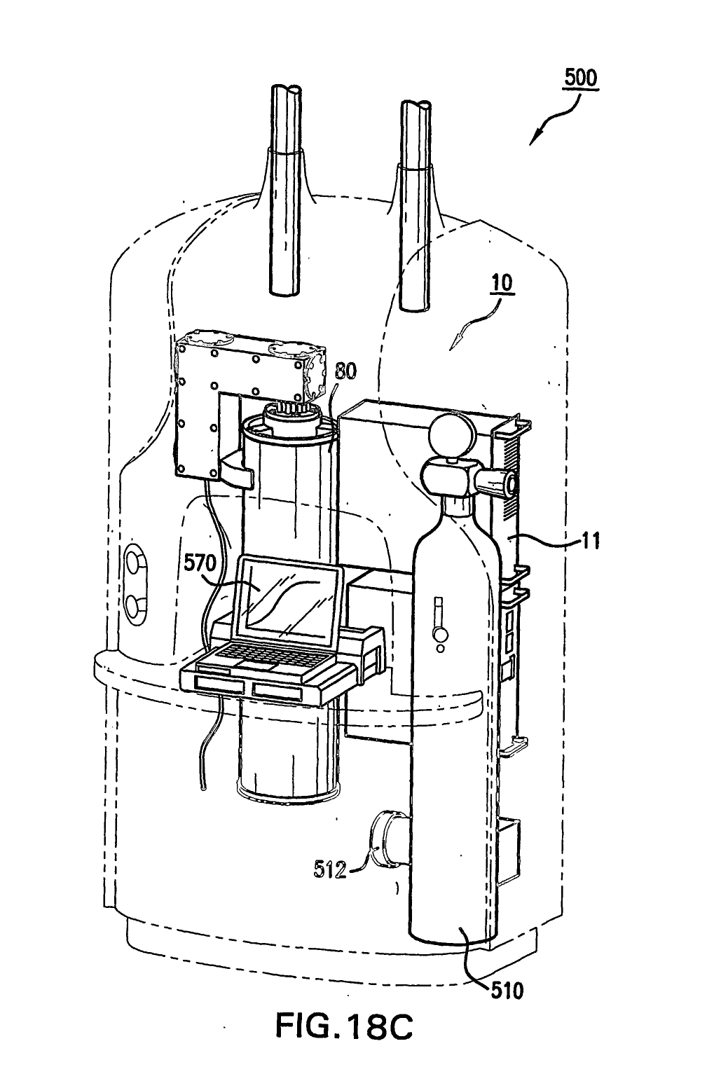

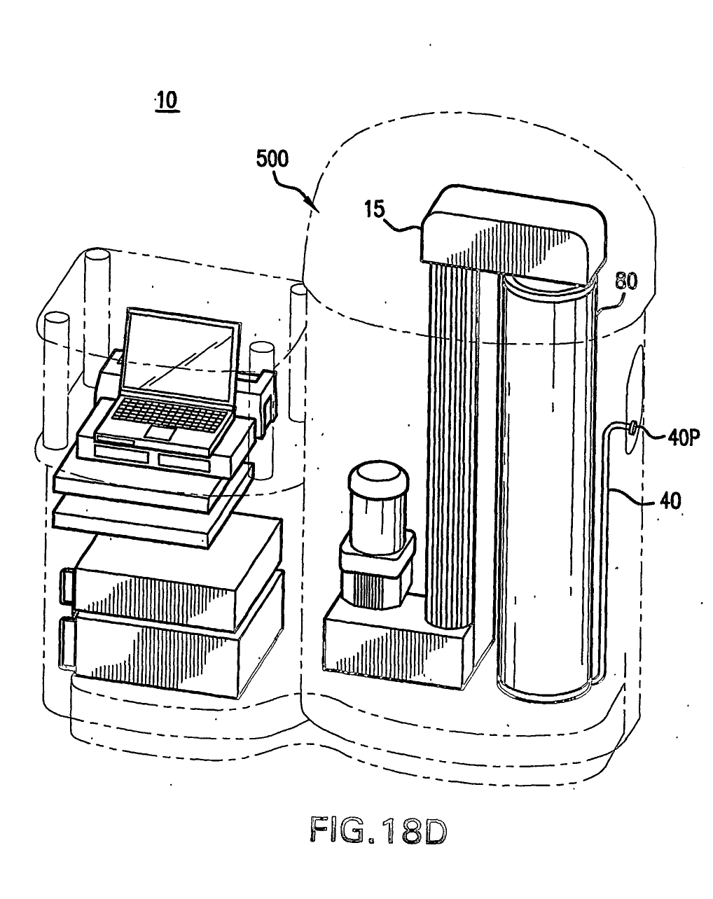

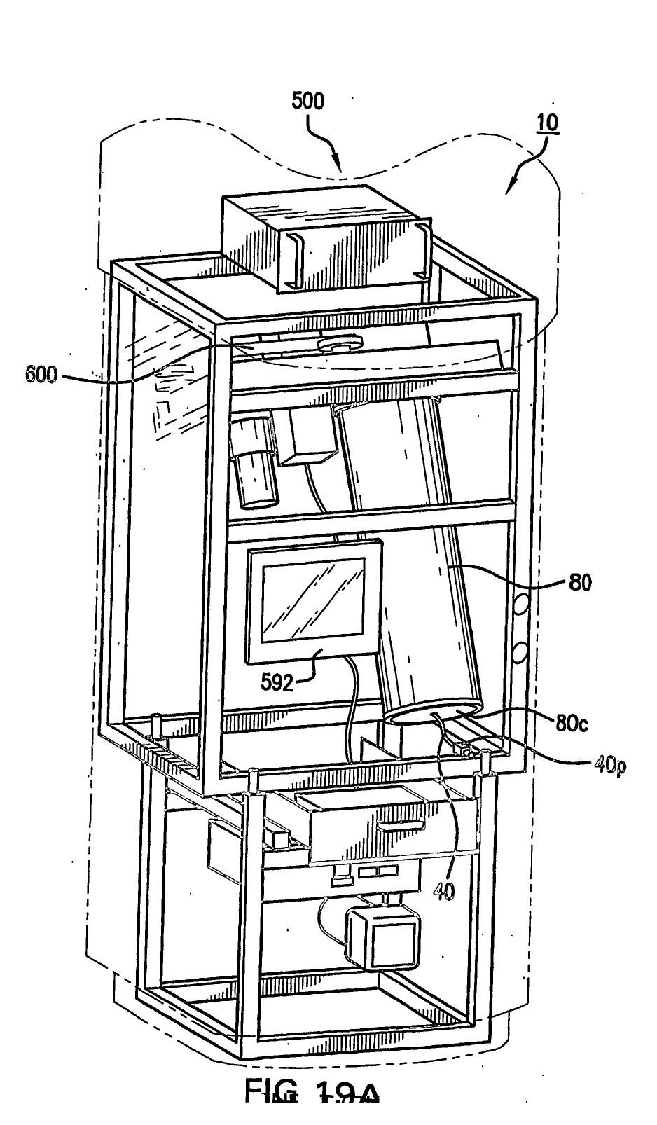

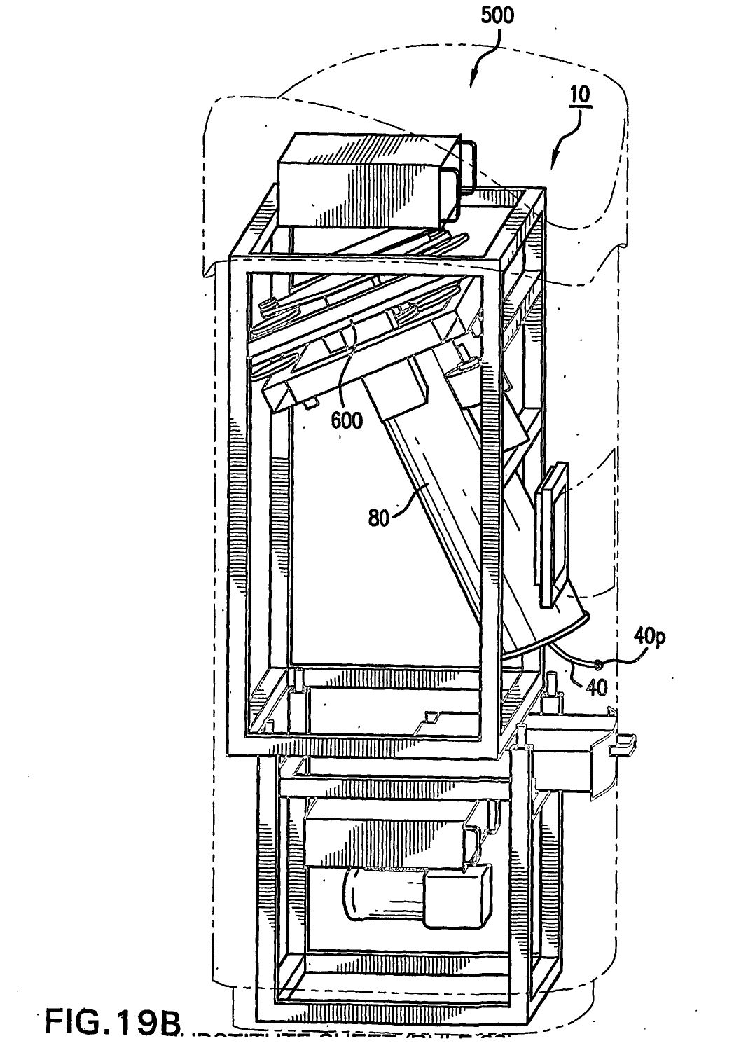

Thus, the field source may be a pair of Helmholtz coils as is well known to those of skill in the art and/or permanent magnets. In certain embodiments, the field source is a cylindrical solenoid 80 (FIGS. 18A, 19A, and 21) that is configured to generate the magnetic field. The solenoid 80 can include a cavity 80c that is sized and configured to surround the optical pumping cell or cells 20 and/or holding cells 30. The polarized gas can be dispensed by directing the gas to flow or dispense from the hyperpolarizer substantially along the axis of the solenoid (FIG. 19B). Suitable solenoid field sources, homogeneity and configurations are described in co-assigned, co-pending U.S. patent application Ser. No. 09/333,571, and permanent magnet configurations are described in U.S. patent application Ser. No. 09/583,663; the contents of these applications are hereby incorporated by reference herein as if recited in full herein. In certain embodiments, the holding cells 30, optical pumping cell or cells 20, and a gas transfer mechanism 300 (more fully described for FIGS. 16,17) are all held within a region of sufficient homogeneity within a single common magnetic holding field B_{H} within the cavity of the solenoid (FIG. 17F). In other embodiments, a plurality of separate magnetic field sources or generators (all electromagnets, all permanent magnets, or combinations of each) can be used, to provide the desired holding fields for the hyperpolarizer (not shown).

For embodiments of the present invention in which the pumping cells 20 and the holding cells 30 are maintained stationary, the target gases may be conducted through relatively rigid conduits and tubing through a control valve or manifold arrangement so as to control and conduct such gas transfers. For embodiments of the present invention in which the pumping cells 20 and/or the holding cells 30 are to be moved during operations, it is contemplated that flexible tubing may be employed which will deflect sufficiently to accommodate the movement of the cells. Alternatively, it is also contemplated that each gas pathway leading from a cell terminate at a valve which will cooperatively engage other terminal valves on other gas pathways. For example, the terminal valves may be configured to open only after another terminal valve has been positioned correctly so as to establish fluid communication between the now-connected flowpaths from each cell.

The cells can be configured to be cells for producing the same type of hyperpolarized target gas, typically a noble gas, such as, but not limited to, all ³He modules or all ¹²⁹Xe modules or combinations of desired target gases.

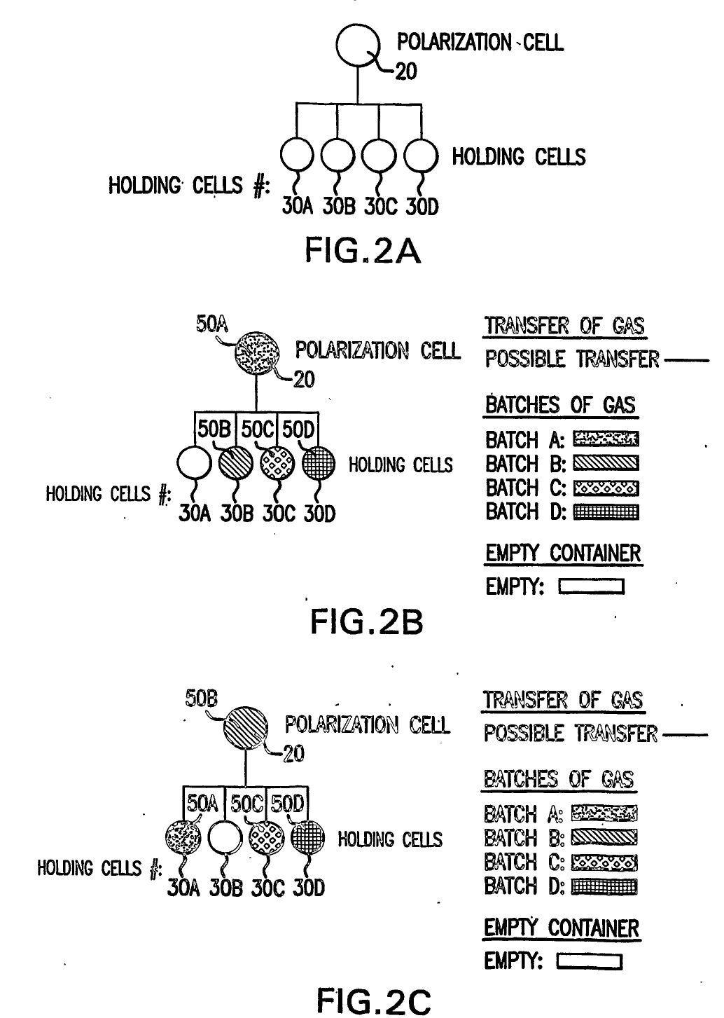

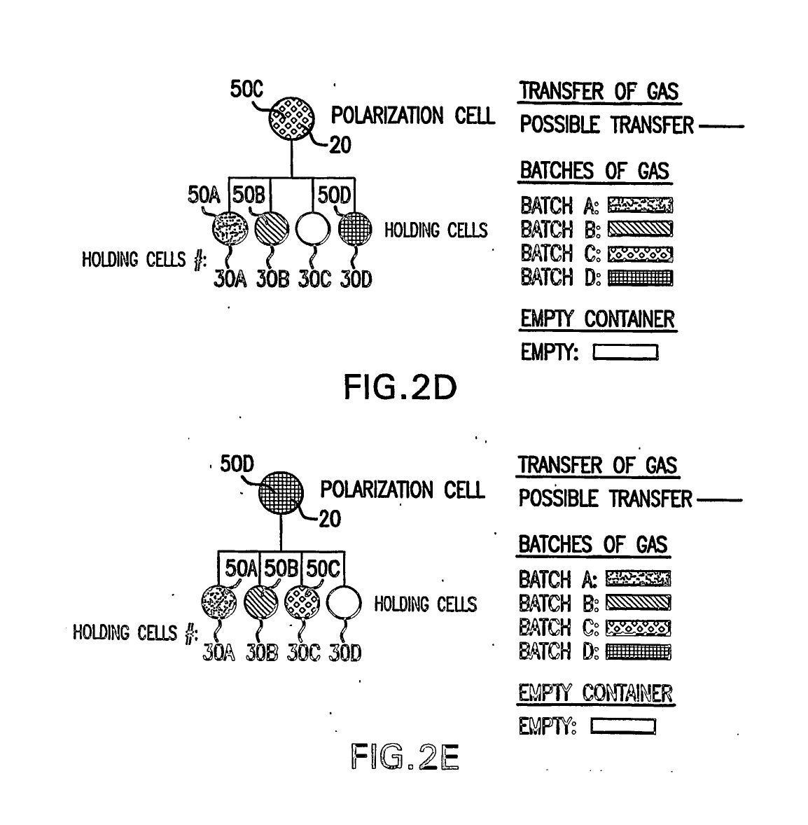

FIGS. 2A-2E schematically illustrate a sequence of operations that can occur using multiple holding cells 30 (shown as cells 30A, 30B, 30C, 30D, each holding a respective batch of target gas 50A-50D) for a corresponding optical pumping cell 20 to produce respective batches of hyperpolarized gas according to embodiments of the present invention. Before or during initial start-up, the holding cells 30 and/or optical pumping cell 20 can be pre-filled or pre-charged with target gas (typically a gas mixture) so that the cells 30 are above ambient pressures, typically at about 110 psi at room temperature (before and/or after polarization). The cells 30 may be configured to hold between about 1-5 liters of unpolarized and/or polarized target gas. Typically, the cells 30 are pre-filled with about 1-3 liters of target gas. Gas transfer mechanisms that create pressure differentials can be used to transfer all or meted amounts of gas from the holding cells 30 to the optical pumping cell 20 as will be discussed further below.

In certain particular embodiments, instead of pre-filling the cells, the cells can be filled with the desired target gas by directing a supply of exogenously held gas into the cells, such as by using the dispensing path and/or port or a fill port and path (not shown). See co-pending, co-assigned U.S. patent application Ser. Nos. 09/949,394; 10/277,911; 10/277,909; and U.S. Provisional Application Ser. No. 60/398,033 (describing manifolds and filling and dispensing systems, as well as purge and evacuate procedures), the contents of which are hereby incorporated by reference as if recited in full herein.

To recharge the cells 30 after dispensing all or portions of a batch of polarized gas, the system can be configured to allow exogenous refills, such as by flowing target gas into the cell or cells and/or replacing selected “used” cells with pre-filled new cells. For applications employing all optical pumping cells, the same pre-filling and/or charging procedures can be used.

Turning back to FIG. 2A, at initiation, none of the batches of target gas are polarized. As shown in FIG. 2B, after each of batches 50A-D have been polarized one, batch 50A is taken from cell 30A, placed in the polarization or optical pumping cell 20 and polarized. Cell 30A is substantially empty during the polarization process. FIG. 2C illustrates that batch 50B is polarized while polarized batch 50A is held in cell 30A. In operation, any of batches 50B-D can be selected for polarization as polarized batch A is held in its cell 30A. FIG. 2D illustrates that batch C is polarized with polarized batches A and B held in their respective cells 30A, 30B, and unpolarized batch 50D awaits polarization. Batch 50D can then be polarized as shown in FIG. 2E. Alternatively, batch 50A can be repolarized and batch 50D polarized as desired, if batch 50A depolarizes below a certain value while the other batches 50B-C are being polarized and before being dispensed for use. One of ordinary skill in the art will also appreciated that this same process may be carried out with unpolarized gases located in any of cells 30A-D so as to allow each such unpolarized gas to be sequentially transferred to pumping cell 20, where it is polarized, and then returned to its respective starting holding cell.

FIG. 3A illustrates the polarization level of different batches of target gas in the optical pumping cell 20 over time, assuming batches 50A-D are polarized in that order. As shown, the polarization strength increases during the polarization procedure (see also FIG. 31). For a hyperpolarizer having four batch cells, the polarization may take about 24-48 hours, and typically about 36 hours, to produce all four (approximately 0.5-1.5 liter sized) batches of polarized gas and bring the polarizer to full operational capacity. Of course, the time to reach full operational status can vary depending on the size and number of batches produced, the dispensed volumes, the wattage used to optically pump the target gas and the desired polarization levels so produced.

FIGS. 3B-3E illustrate examples of different decay profiles for each of the respective batches 50A-D (polarization strength over time) now provided by the hyperpolarizers of the present invention. Although the portion of the polarization line shown relative to holding cells not related to the batch shown in FIGS. 3B-3E is shown as straight (and close to the x-axis), it will be appreciated that the polarization level in each or certain cells can hold polarized gas, and thus, each cell can have a steady state polarization decay (similar to that shown in FIG. 3G). Of course, not all cells are required to be filled or used and certain ones may be empty or unused in certain embodiments.

In any event, the polarization level can be different in different holding cells 30, at any one time. As shown, the system can be sized so that at fall capacity batch A is ready to be repolarized upon polarization of batch D. FIG. 3F illustrates that at any given time during the monitoring period, there is gas polarized to a desired level available to a user. The system 10 can be configured to selectively dispense the batch or a portion of the batch that is at or above the desired level and/or provide multiple different portions of different batches as desired.

FIGS. 4A-4B illustrate another embodiment of the present invention that is similar to the embodiments shown in FIGS. 1D and 1E. As shown, the optic system 15 includes a laser source 16 that generates an unpolarized laser beam. The unpolarized laser beam is optically processed (using mirrors, lenses, quarter wave plates 15w and the like) to provide horizontally and vertically polarized beams, 15H, 15V, respectively. FIG. 4A illustrates that the vertically polarized and horizontally polarized beams 15H, 15V can be used to concurrently optically pump respective polarization or optical pumping cells 20A, 20B. FIGS. 4B and 4C illustrate that the polarized beams 15H, 15V can converge upon different optical pumping cells as light beam 15L to serially pump selected cells. FIG. 4B illustrates that the beam 15L is directed to cell 20A and FIG. 4C illustrates that the beam 15L can then be directed to cell 20B.

Generally described, in operation, the optical pumping cell or cells 20 are heated to an elevated temperature, generally to about 170-200° C. or greater. The target gas mixture is preferably introduced into one of cells 20A-C at a pressure of between about 6-10 atm. Of course, as is known to those of skill in the art, with hardware capable of operating at increased pressures, operating pressures of above 10 atm, such as about 20-30 atm, can be used to pressure-broaden the alkali metal absorption and promote optical pumping. Using increased pressures with an alkali metal (such as rubidium (“Rb”)) can facilitate the absorption of the optical light (approaching up to 100%). In contrast, for laser line widths less than conventional line widths, lower pressures can be employed.

The optical pumping cells 20 typically include a quantity of alkali metal that vaporizes and cooperates to provide the spin-exchange polarization of the target gas of interest. The alkali metal can typically be used for a plurality of pumping procedures without replenishment. The optical pumping cell has conventionally been formed from a substantially pure (substantially free of paramagnetic contaminants) aluminosilicate glass because of its ability to withstand deterioration due to the corrosive potential of alkali metal and its relatively friendly treatment of the hyperpolarized state of the gas (i.e., “good spin relaxation properties”—so stated because of its ability to inhibit or retard surface contact-induced relaxation attributed to collisions of the gas with the walls of the cell). Coatings such as sol-gel coatings, deuterated polymer coatings, metal film coatings and other coatings and materials that inhibit depolarization have also been proposed. See, e.g., U.S. patent application Ser. No. 09/485,476 and U.S. Pat. No. 5,612,103, the contents of which are hereby incorporated by reference as if recited in full herein.

During polarization, the noble gas of choice (conventionally ³He or ¹²⁹Xe) is held in the optical cell along with the alkali metal. The optical pumping cell is exposed to elevated pressures and heated in an oven to a high temperature as a light source, typically provided by a laser and/or laser array in an optic system, is directed into the optical cell to optically pump the alkali metal and polarize the target gas.

Hyperpolarizer systems of the present invention may employ helium buffer gas in the optical pumping cell 20 to pressure broaden the Rb vapor absorption bandwidth. The selection of a buffer gas can be important because the buffer gas—while broadening the absorption bandwidth—can also undesirably impact the alkali metal-noble gas spin-exchange by potentially introducing an angular momentum loss of the alkali metal to the buffer gas rather than to the noble gas as desired.

As will be appreciated by those of skill in the art, Rb is reactive with H₂O. Therefore, any water or water vapor introduced into the polarizer cell 20 can cause the Rb to react and decrease the rate of spin-exchange in the polarizer cell 20. Thus, as an additional precaution, an extra filter or purifier (not shown) can be positioned before the inlet of the polarizer cell 20 with extra surface area to remove even additional amounts of this undesirable impurity in order to further increase the efficiency of the polarizer.

Hyperpolarizer systems of the present invention can also capitalize on the temperature change in the outlet line between the heated pumping cell 20 and the holding cell 30 to precipitate the alkali metal from the polarized gas stream in the cell 20 and/or in the conduit proximate the cell 20 that forms a part of the gas flow path. In other embodiments, the cell 20 itself can be cooled down to recapture the Rb or other alkali metal before the polarized gas is allowed to exit from the cell 20. The hyperpolarizer 10 can be configured with active rapid cooling of the optical pumping cell 20 by flowing coolant (such as cold air) into the oven and directing hot air out. The rapid cooling may occur within between about 5-40 minutes after polarization is complete and the laser is no longer optically pumping the cell 20. In certain embodiments, the rapid cooling is carried out in under about 15 minutes, and in particular embodiments, in about 5-10 minutes. Thus, for multi-holding cell embodiments, after cool-down the polarized target gas can be returned to its holding cell 30. The elevated heat may be supplied to the oven by directing hot air to flow therein in a recirculating oven configuration and holding the heating element in a remote location to inhibit any depolarizing influences from proximity of the gas. In other embodiments, the laser energy is captured in a thermally insulated oven space, providing a substantially self-heating configuration that harnesses the heat released by the optical pumping process.

As will be appreciated by one of skill in the art, the alkali metal can precipitate out of the gas stream at temperatures of about 40° C. The unit 10 can also include an alkali metal reflux condenser (not shown) or post-cell filter (not shown). The refluxing condenser can employ a vertical refluxing outlet pipe, which is kept at room temperature. The gas flow velocity through the refluxing pipe and the size of the refluxing outlet pipe is such that the alkali metal vapor condenses and drips back into the pumping cell by gravitational force. Alternatively, and/or in addition, a Rb filter can be used to remove excess Rb from the hyperpolarized gas prior to collection or accumulation along the dispensing path 40 or at the dispensing port 40p (FIGS. 1A, 1B). In any event, it is desirable to remove alkali metal prior to delivering (and typically, prior to dispensing from the hyperpolarizer) the polarized gas to a patient to provide a non-toxic, sterile, or pharmaceutically acceptable substance (i.e., one that is suitable for in vivo administration).

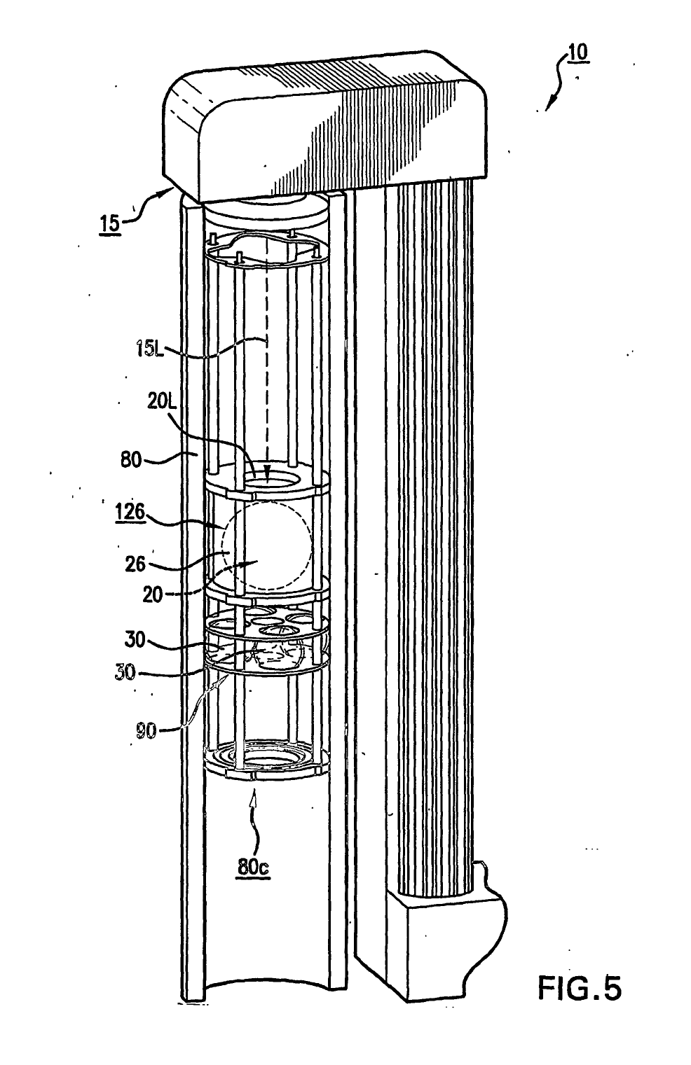

Turning now to FIG. 5, one embodiment of a hyperpolarizer 10 having a plurality of holding cells 30 and a single optical pumping cell 20 is shown. In this embodiment, the optical pumping cell 20 is disposed above the holding cells 30. The holding cells 30 are held in coplanar alignment in the cavity of the solenoid using a mounting assembly 90. The solenoid 80 is shown cut away to illustrate that the optical pumping cell 20 (inside its respective oven 26) as well as the holding cells 30 are configured and sized to be held in the solenoid cavity 80c. The optic system 15 is configured to direct the laser light 15L down toward the optic pumping cell 20 via a light channel 20L formed in the top portion of the oven assembly 126.

FIGS. 6A-6E illustrate one example of an optical pumping cell sub-assembly 95 with an oven assembly 126. As shown, the sub-assembly 95 includes the oven assembly 126 which comprises the optical pumping cell 20, an RF NMR (surface) coil 93 positioned to be in communication with the cell 20 and a heater 26o with leads 26e. The cell 20 sits on a support holder 99 that resides on the heater 26o that provides heat to the cell 20 and oven during operation. As noted above, the heater 26o may be located remotely and hot air delivered to the oven assembly 126 and/or no heater may be required at all if the laser energy is captured sufficiently to provide a self-heating arrangement.

As described above, FIG. 6F is a schematic illustration of a thermally insulated cavity 27 that can use the laser beam to provide at least a portion of the heat used to elevate the optical pumping cell 20 and that uses a cooling source 27c that can cool the insulated cavity to maintain the insulated cavity, and hence the gas, at desired controlled temperatures and/or to actively cool the cell 20 in the cavity 27c after polarization.

Still referring to FIGS. 6A-6E, the sub-assembly 95 also includes an inner wall 26w₁ and outer wall 26w₂ with a stack of thermal insulator discs 20d held therebetween. The stack of discs 20d may define the inner and/or outer wall and the use of a separate wall component is not required (not shown). The sub-assembly 95 further includes opposing top and bottom housing members 20t, 20b with columns 96 that extend between the top and bottom housing members 20t, 20b and secure the same together in spaced apart alignment. The top housing member 20t and discs 20d are configured with a light passage 20L that allows the laser light to enter therein during polarization. The optical pumping cell 20 includes an elongate capillary stem segment 20s (FIG. 6A) that extends through a central aperture 21 formed in the lower components 97w, 97a, 97b of the sub-assembly 95. The lower components 97w, 97a, 97b are matably configured to attach together and provide a support base for the stacked discs 20d, the cell 20, and the inner and outer walls of the oven 26w₁, 26w₂.

Oven heat and/or cool-down ducting 201, 202 can provide convective and/or conductive heat transfer for heating and/or cooling the thermal space and/or to achieve rapid forced-air or forced-coolant cooling of the thermal space or oven, before, during, or after polarization is complete. The target gas flow paths (30f, FIG. 1A) are not shown but can be configured to be in fluid communication with the stem 20s of the cell. Typically, a flow tube or conduit will enter a distance into the aperture 21 proximate the bottom member 20b and sealably engage with the stem 20s of the cell 20. Duct 210 will accommodate these conduits (not shown). This target gas flow tube from the optical pumping cell 20 defines a portion of the gas flow path 30f (FIG. 1A). The flow tube is operably associated with valves and controls to allow control of the direction of flow of gas into and out of the cell 20. The flow path 30f can also be configured to selectively engage with a purge gas source (such as a nitrogen cylinder or other desired purge gas) and evacuation path to remove contaminants in the polarized gas flow path. As such, the respective gas flow paths can include valves that control the selection of the gas introduced into the flow lines, the direction of flow, and the like. Other configurations, shapes, and/or sizes of ovens, optical pumping cells, holding cells, mounting assemblies, heaters, purge and evacuate flow paths and the like may be employed.

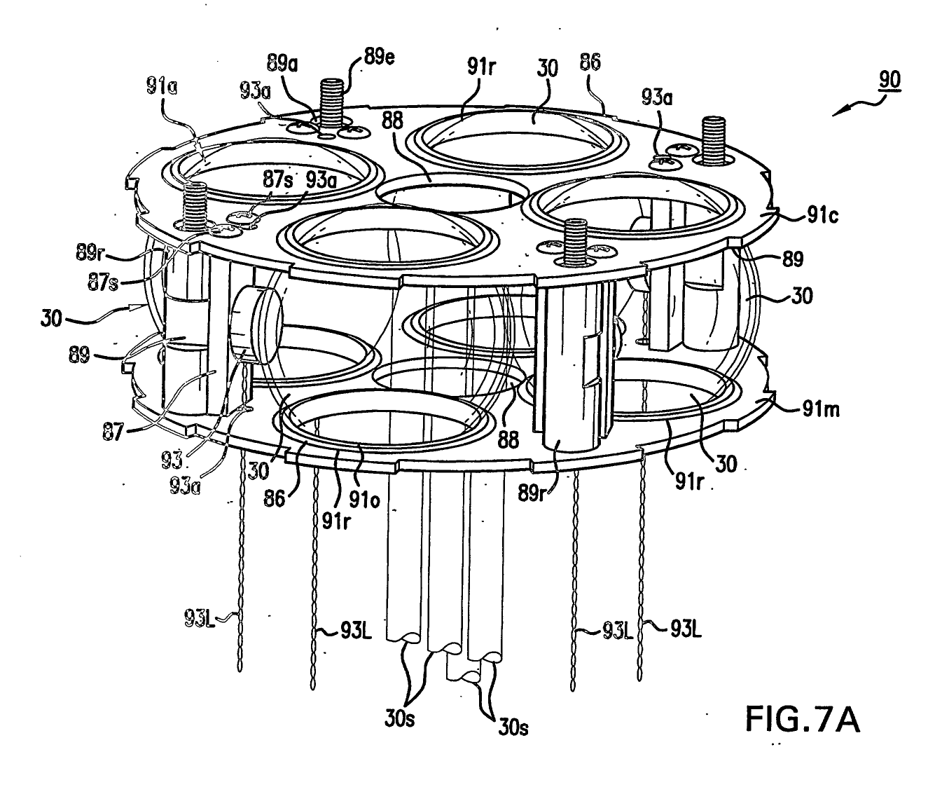

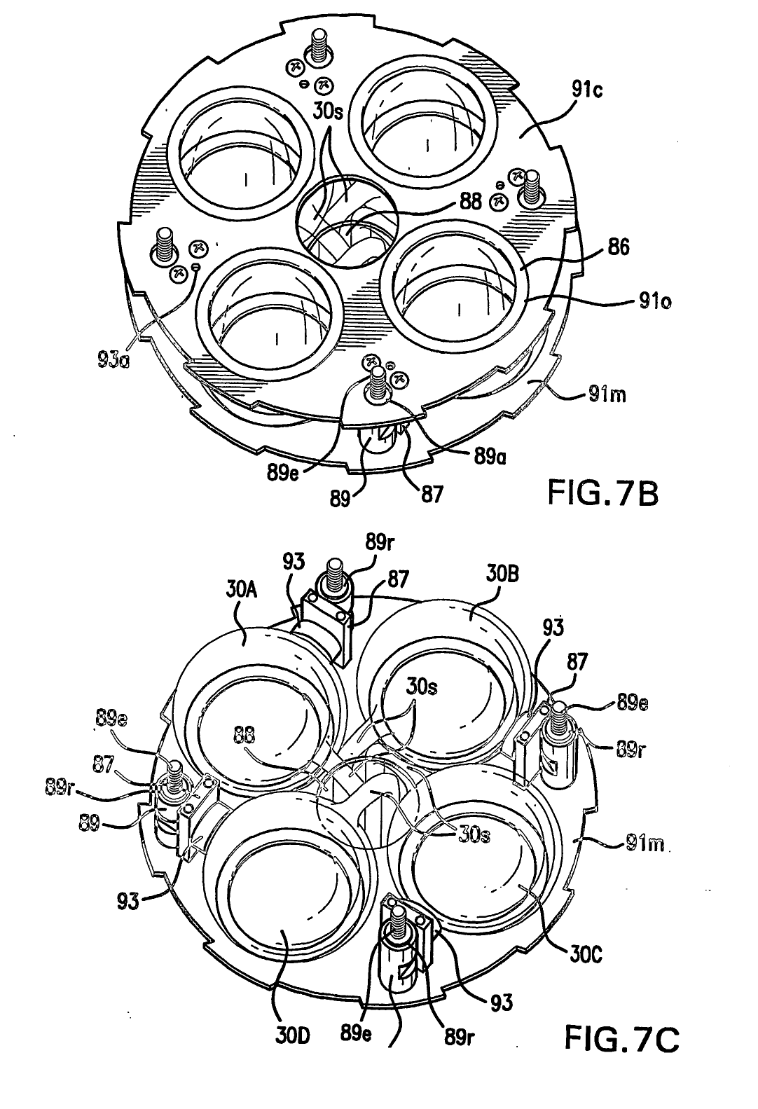

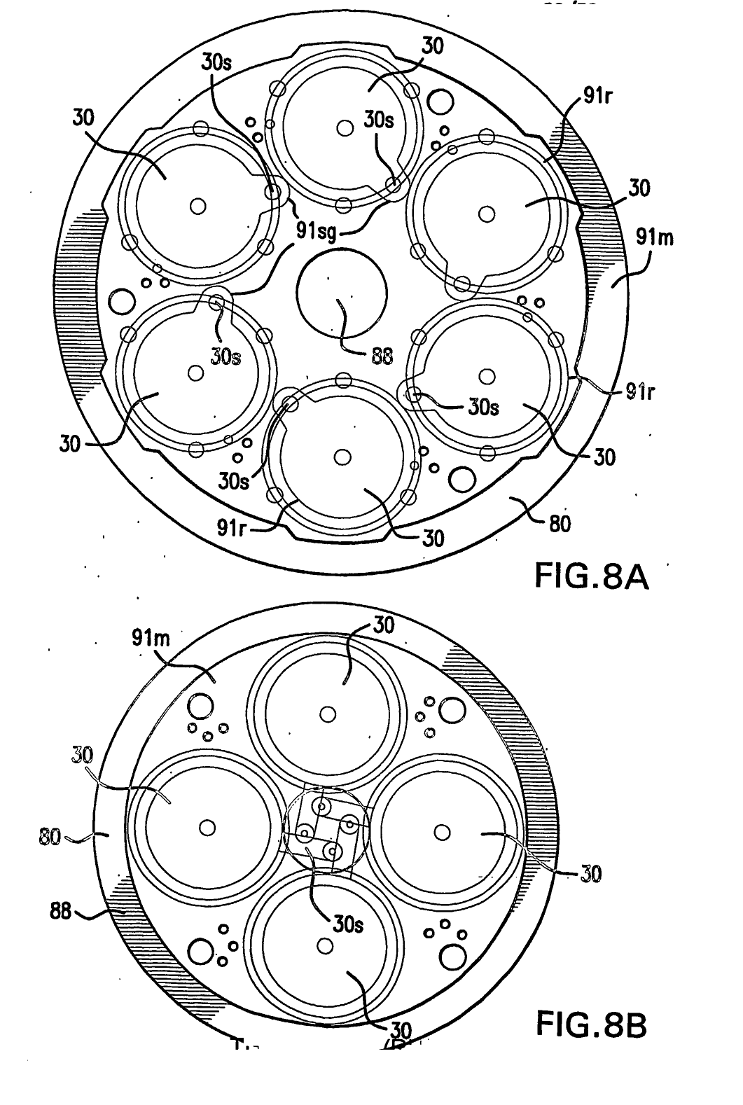

FIG. 7A illustrates one example of a holding cell mounting assembly 90. As shown, the mounting assembly 90 includes a mounting plate 91m with spaced apart mounting regions 91r, each region 91r configured to releasably hold a respective holding cell 30 therein or thereon. The cells 30 can each include an elongate capillary stem 30s and the mounting plate 91m is configured to allow the stems 30s to extend therethrough. In the embodiment shown, the mounting plate 91m includes a substantially central aperture 88 that is sized and configured to allow all of the stems 30s to extend therethrough. The stems 30s are shown as extending in a substantially downward direction, but other directions may also be suitable depending on the orientation of the magnetic field and the relative position of the optical pumping cell 20 and/or dispensing port 40p of the hyperpolarizer systems of the present invention. FIG. 8A illustrates the stems 30s extending through separate apertures rather than a common central aperture 88. In the embodiment shown in FIG. 8A, the central aperture 88 can be used to allow a different portion of the gas flow path to extend therethrough.

Turning back to FIG. 7A, the assembly 90 may also include a cover plate 91c. The cover plate 91c maybe configured to be the same as the mounting plate 91m, for ease of assembly and production. However, the cover plate 91c may also be configured differently from the mounting plate 91m. In any event, a plurality of mounting columns 89 can be used to space and secure the cover plate 91c and mounting plate 91m about the holding cells 30. The columns 89 may be used to support the RF coils 93 and each holding cell 30. The columns 89 may be sized and configured to allow the cover and mounting plates, 91c, 91m to rest on opposing upper and bottom portions 89r but may include extension portions 89e (shown as threaded in the upper direction above the cover plate 91c) that may extend through column apertures 89a formed in the plates 91c, 91m. The extension portions 89e may be used to attach to other column segments (FIG. 5) to position the mounting assembly with holding cells in a desired space within the hyperpolarizer 10. The lower portion of the column may include female threads while the upper portion includes male threads (as shown); reverse configurations or alternative attachment devices can also be used.

As shown, a spacer bracket 87 is attached to respective columns 89 and the bracket 87 holds the NMR coils 93 in close proximity (typically contacting) to the outer surface of the cell 30 holding the target gas 50. The spacer brackets 87 may be attached from the top and/or bottom using threaded members 87s. The spacer brackets 87 may also be attached to the sides of the columns or otherwise positioned in the assembly 90. The mounting plate 91m may also include a plurality of NMR coil lead apertures 93a that allow the NTMR leads 93L from the respective NMR coils 93 to extend through the mounting plate 91m. The NMR coil 93 may be positioned in other locations about the cell bodies 30 but should be held so that it is substantially perpendicular to the magnetic field. FIGS. 7B and 7C illustrate other views of the assembly shown in FIG. 7A. FIG. 7C illustrates the assembly without a cover plate 91c.

In the embodiments illustrated in FIGS. 7A-7C, the cells 30 are closely spaced apart. The mounting plate 91m may be substantially circular with the cells 30 positioned to be symmetrically spaced apart about its surface. The mounting plate 81m may include a cell receptacle aperture 91a that is sized and configured to allow a portion of the cell body to extend below (and/or above) the boundary of the surface of the mounting plate 91m and/or cover plate 91c. A resilient ring 91o can be used to help insulate, inhibit, or protect the cells from inadvertent movement.

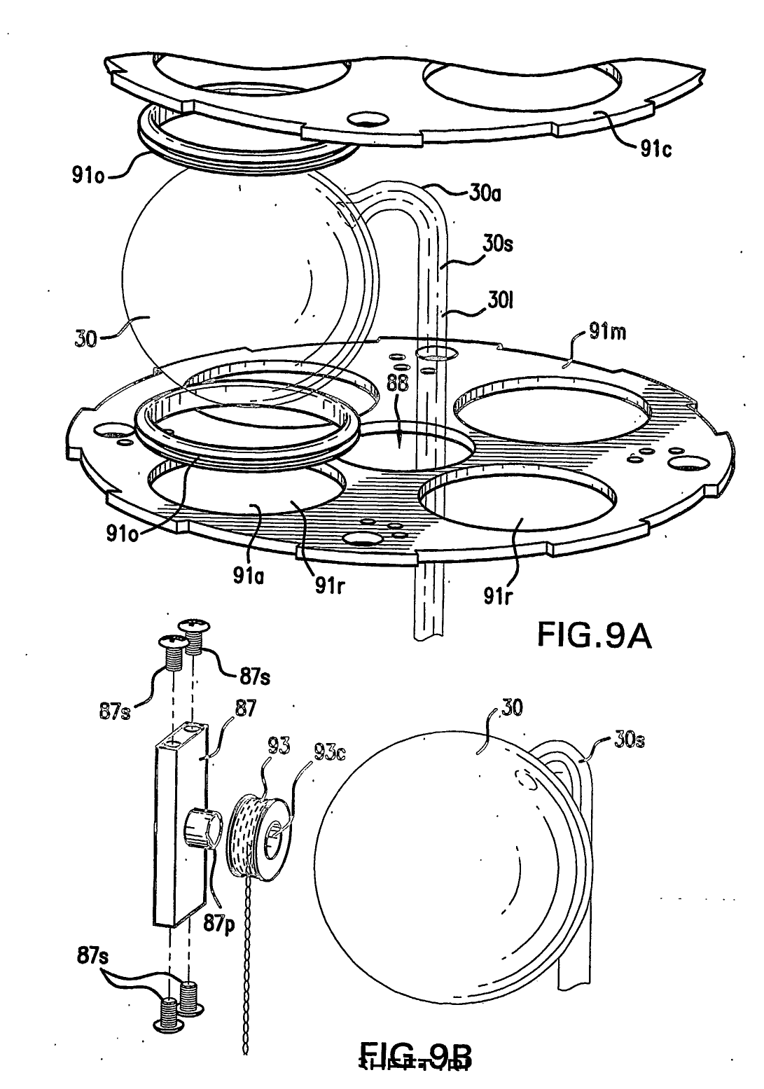

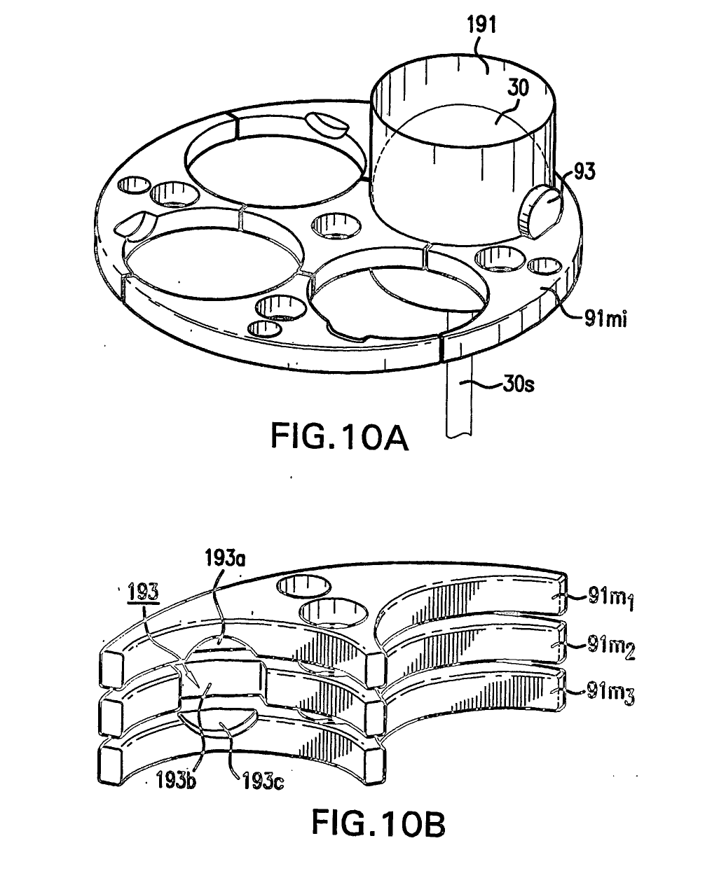

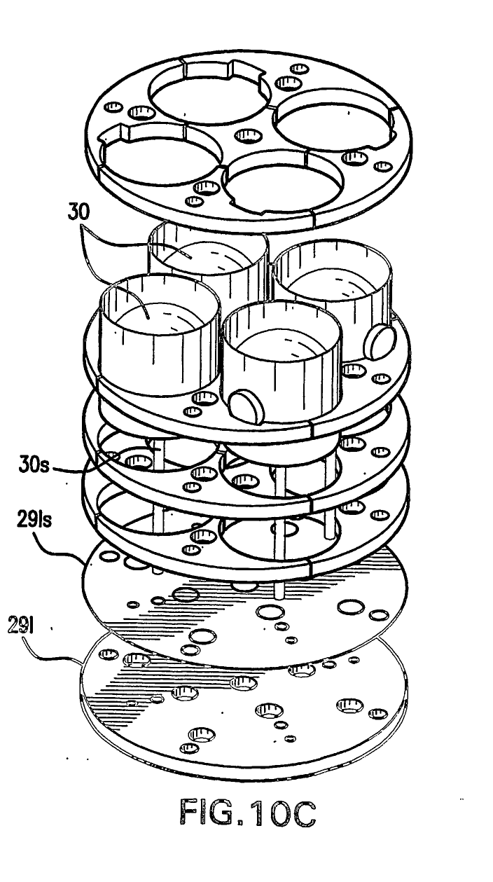

As shown in FIG. 9A, the cell stems 30s can be configured to attach to the body of the cells with an arcuate segment 30a that then terminates into a substantially linear segment 30l. The arcuate segment may be such that the stems 30s extend from the cell body and turn at a substantially 90-degree angle. This configuration allows the stems to extend through a common aperture 88 (FIG. 8B) having an opening size that is less than the cross-sectional width of the cells themselves. Other embodiments provide cells 30 without an arcuate segment but having linear stems 30s (FIG. 10A).

FIG. 8A illustrates a configuration of six cells 30 and FIG. 8B illustrates a four cell 30 arrangement. The nominal distance between cells 30 measured across the centers of adjacent cells may be between about 1-5 inches. As noted above, the mounting plate 91m can be sized to reside in a solenoid cavity 80c (FIG. 5). The solenoid cavity 80c may be on the order of 6-20 inches O.D., with a 0.25-0.75 inch wall thickness, and an I.D. on the order of 5.25-19.75 inches. Other sizes may be suitable in different applications. In certain embodiments, the plate 91m may be sized with a width that is between about 5-18 inches, and typically between about 7-10 inches.

FIG. 8A illustrates that the mounting plate 91m may include a stem segment region 91sg proximate the body of each respective cell 30. The stem segment region 91sg may be rotated about the perimeter space of holding region 91r, so that each stem segment 91sg is oriented differently from the others, and is closer or farther from the outer perimeter of the mounting plate 91m.

It is noted that the cells 20, 30 are illustrated as substantially spherical. This configuration provides a suitably low volume to surface area ratio to help inhibit contact-induced depolarization. However, other shapes and configurations of cells may be employed. It is noted that the substantially spherical cell bodies may have variation that can influence their mounting. As such, the mounting configurations may be designed to be sufficiently adjustable or accommodating to accept typical variation in sizes and/or shapes.

FIG. 9A illustrates a single cell 30 in position for mounting on the mounting plate 91m. As shown, the holding cell 30 includes a stem 30s with an arcuate portion 30a and a linear portion 30l. FIG. 9B illustrates the RF coil 93 with a center opening 93c configured to attach to a projection surface on spacer bracket 87. Again, threaded members 87s (or screws) may be used to attach the bracket 87 to the mounting plate 91m and/or cover plate 91c.

FIGS. 10A-10C illustrate an alternative insulating configurations for holding the cells 30. As shown, the mounting plate can be an insulating plate 91mi that is sized and configured to receive a holding member 19. The holding member 191 is sized and configured to snugly abut a respective cell 30 and position the NMR coil 93 in abutting contact with the cell body 30. The holding member 191 may be open on both ends as shown. The stem 30s of the cells 30 are shown as oriented to extend substantially downward. The plate 91m can be a plurality of stacked plates 91m₁, 91m₂,91m₃ that, in position, are in closely spaced or abutting contact. The stacked plates each include a portion 193a, 193b, 193c, of a receptacle 193 sized and configured to hold the outer perimeter of the NMR coil 93 therein. FIG. 10C illustrates four stackable plates with respect to the holding cells 30 and holding members 191 over a lower housing member 291 and associated spacer 291s that matably attach to the stacked plates and allow the cell stems 30s to extend therethrough.

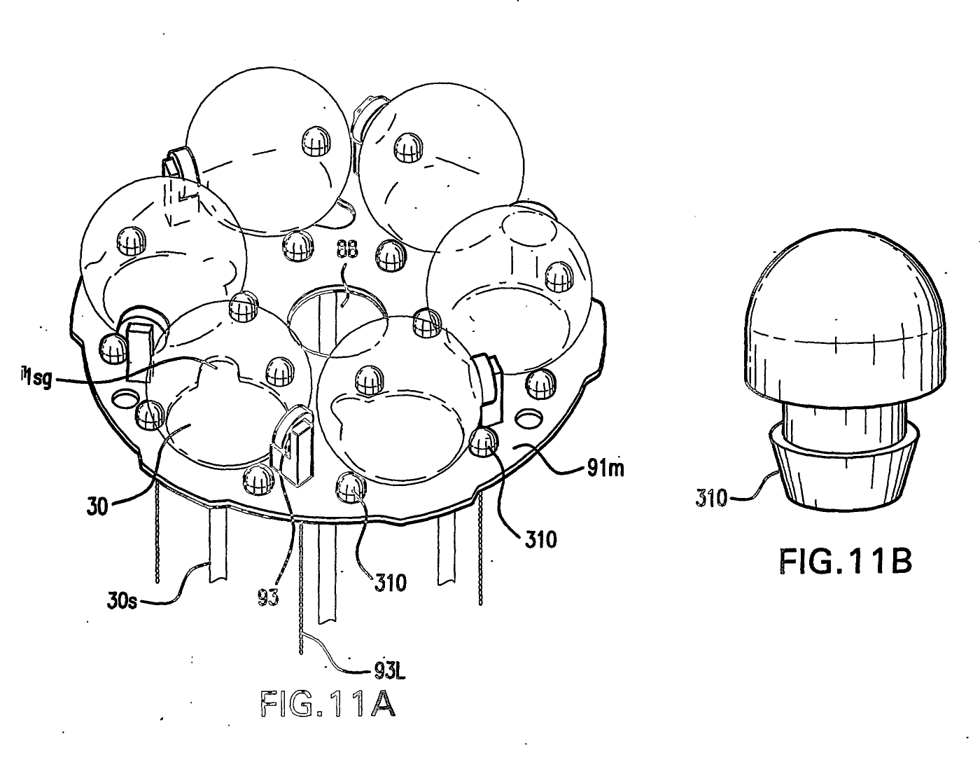

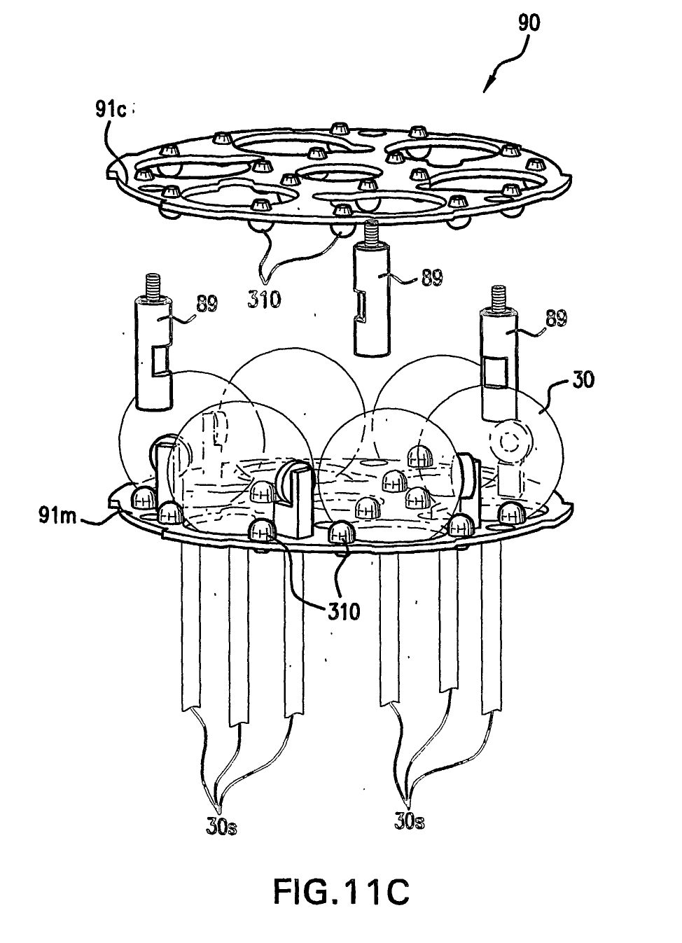

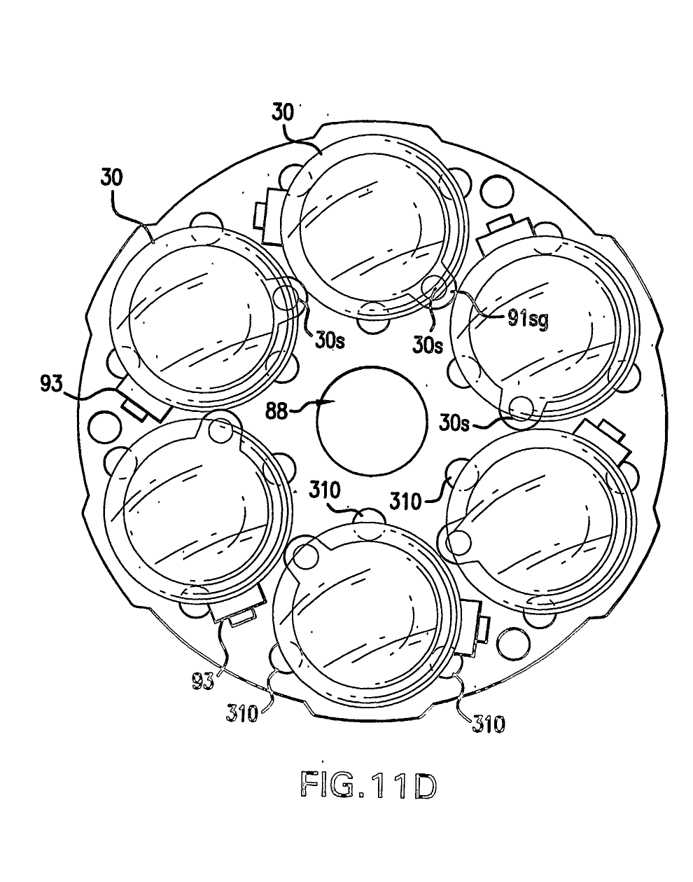

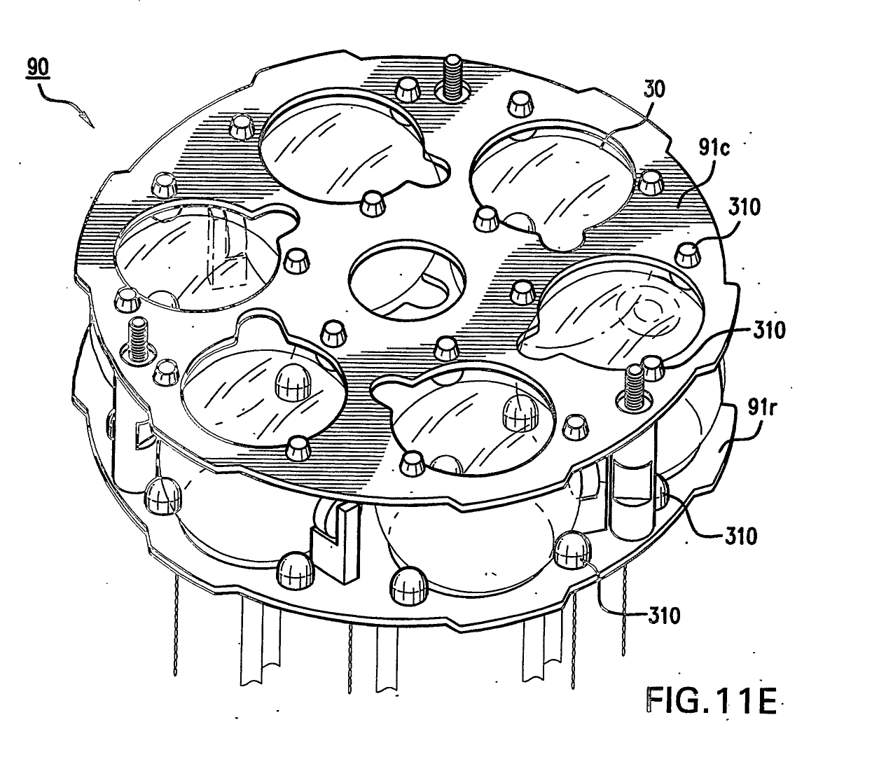

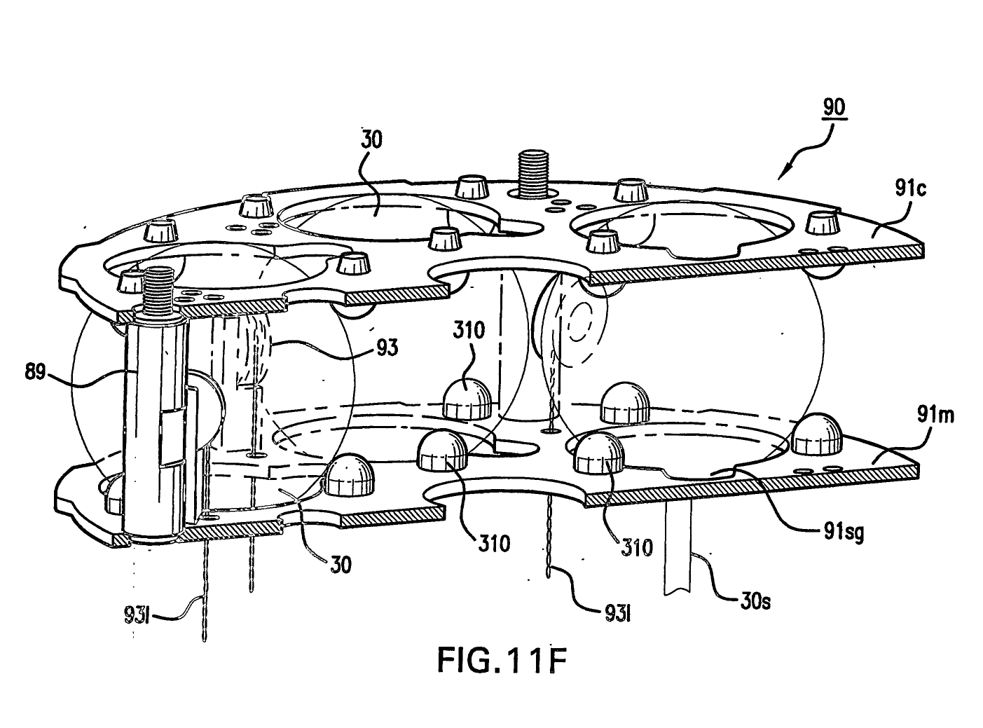

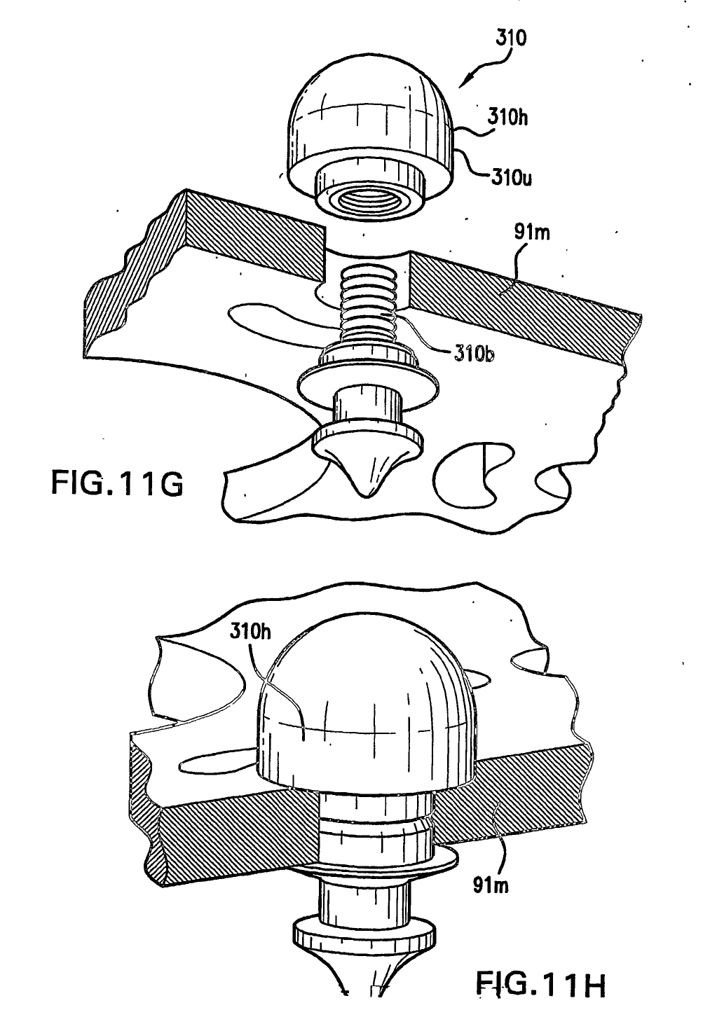

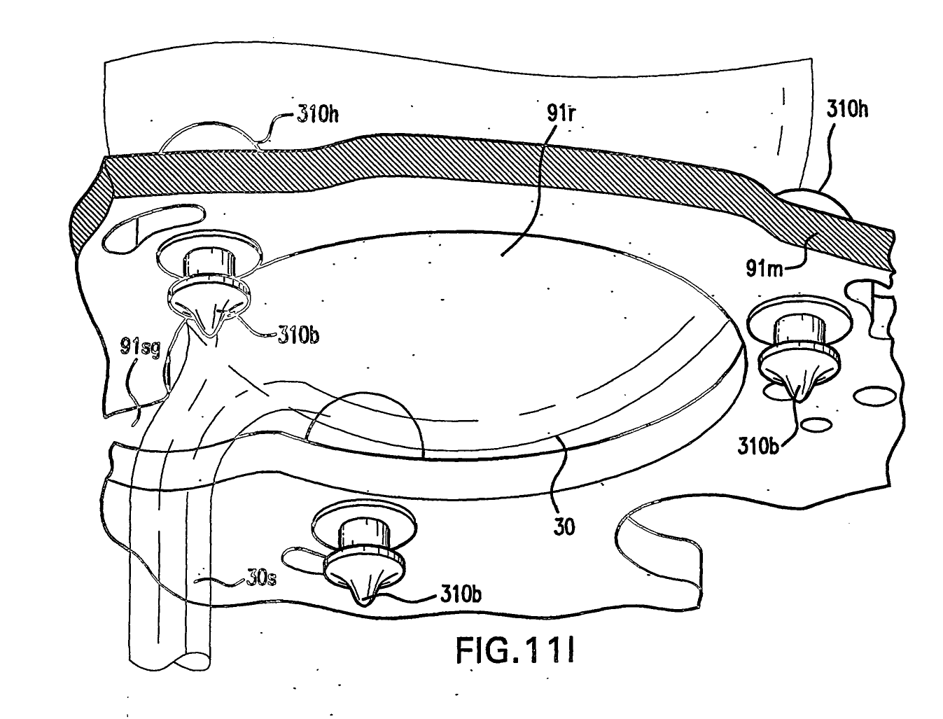

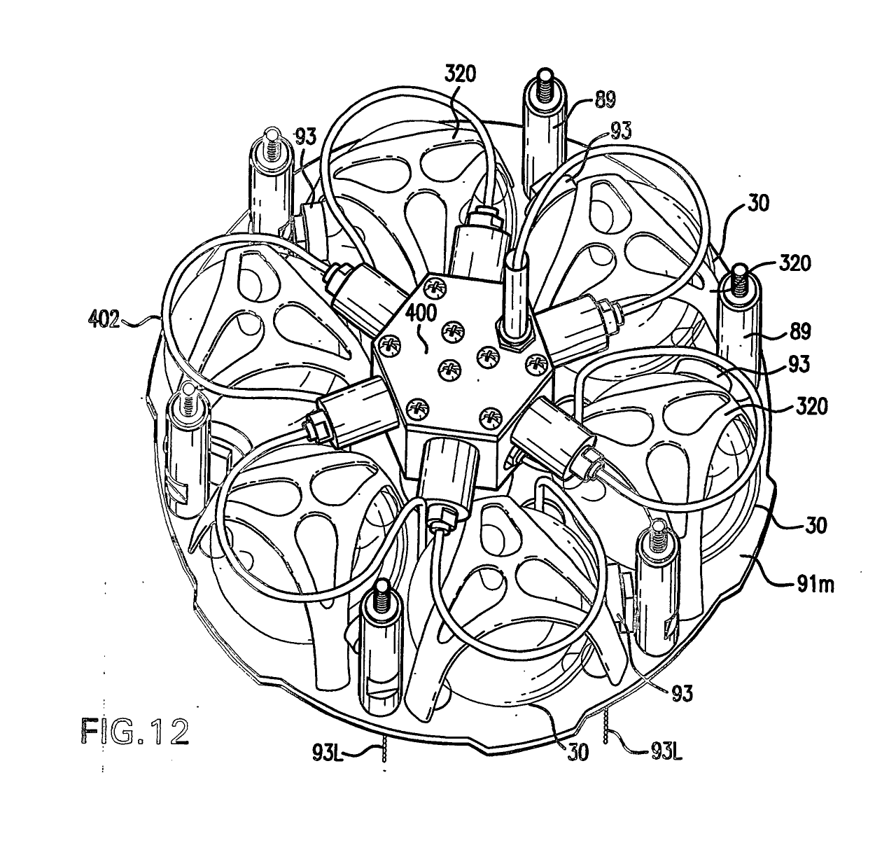

FIGS. 11A-11F illustrate yet another embodiment of a mounting assembly 90. As shown, the holding cells 30 are held in position on the mounting plate 91m using a plurality of resilient nipples 310 that are spaced apart about the perimeter of the cell body on the mounting plate 91m. FIG. 11B illustrates that the nipple 310 may be a resilient unitary member that can be press fit (frictionally engaged)into the plate 91m so that its head 310h rises above the surface of the plate 91m. The nipples are positioned between columns 89. The cover plate 91c may be configured with corresponding nipples 310 with the heads 310h oriented to extend down to face the heads 310h on opposing sides of the respective cell bodies 30. FIGS. 11G-11I illustrate alternative nipple configurations. As shown, the nipple 310 is configured as a two-piece body, having a resilient head 310h as a first portion and a threaded member as a second portion 310b. The two members 310u, 310b attach on opposing sides of the plate 91m with the threaded portion extending into the head 310h.

FIG. 12 illustrates yet another embodiment of a mounting assembly 90. In this embodiment, elastomeric straps 320 are used to releaseably secure the cells 30 to the mounting plate 91m. The elastomeric straps 320 anchor to plate 91 about each cell 30.

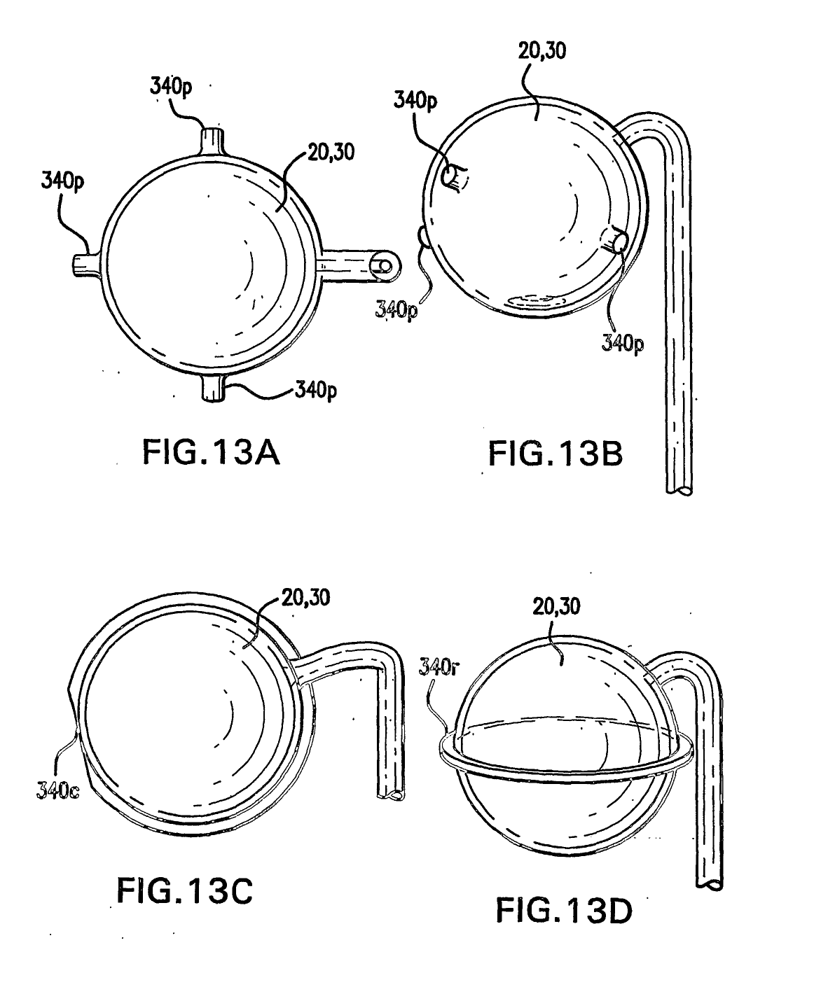

FIGS. 13A-13D illustrate examples of cell bodies with perimeter surfaces having alignment shapes formed integrally thereon. FIGS. 13A and 13B illustrates a cell body outer surface with a plurality of outwardly extending projections 340p. FIG. 13C illustrates a planar region 340c that is formed into the body, while FIG. 13D illustrates a ridge such as an annular ring formed into the outer surface of the cell body. The alignment shapes on the cell bodies may be used alone or in combination with the mounting guides proposed above or otherwise desired in order to hold the cells in position while accounting for variation in shape of the cell, cell to cell. Additionally, these features can be used to orient the NMR surface coil 93 relative to the cell.

FIG. 12 also illustrates a gas distribution valve 400 that can be used to serially select or activate the gas flow paths associated with respective holding cells 30. The gas distribution valve 400 can direct gas from a common port to travel from or to one of a selected plurality of different ports (such as those associated with each holding cell). The common port may direct the gas to a gas transfer station or directly to the optical pumping cell. An example of a suitable gas distribution valve is a gas chromatograph valve or valves are models Valcon E Rotor with a TI body having P/N EMT6CSDL1MWETI-485 and valve model PPS-Stator-Valcon E2 Rotor, P/N C25Z-3180EMHY-485, available from VICIAG, Valco International, located in Houston Tex. The rotor valves function similar to a rotary spool valve (using a combination of two may be helpful, depending on the number of different holding cells). The valves, like the flow paths and cells, should be made out of materials that inhibit depolarization, such as titanium, suitable polymers and the like. Modified or custom valves may also be used. The gas distribution valve 400 will be discussed below with respect to FIGS. 16-17.

For the embodiments discussed above with respect to holding cells 30 described herein, the same or similar configurations and mountings may be used, taking into account modifications for ovens and support accessories where needed, where multiple optical pumping cells 20 are employed. For example, for the embodiment shown in FIG. 7A or 8A, a plurality of ovens 26 can be configured to mount over the respective cells 20. In other embodiments, the optical pumping cells 20 may be translated into the oven during polarization.

In certain embodiments, the mounting plate 91m can be configured to rotate via a drive system connected thereto (not shown) to position one or more selected optical pumping cells in communication with the optic system for polarization and/or repolarization of the target gas held therein. This allows the cells 20 themselves to be statically held on the mounting plate 91m and the plate indexed or translated (rotated back and/or forth) to position the optical pumping cell 20 in the targeted polarization position in the hyperpolarizer.

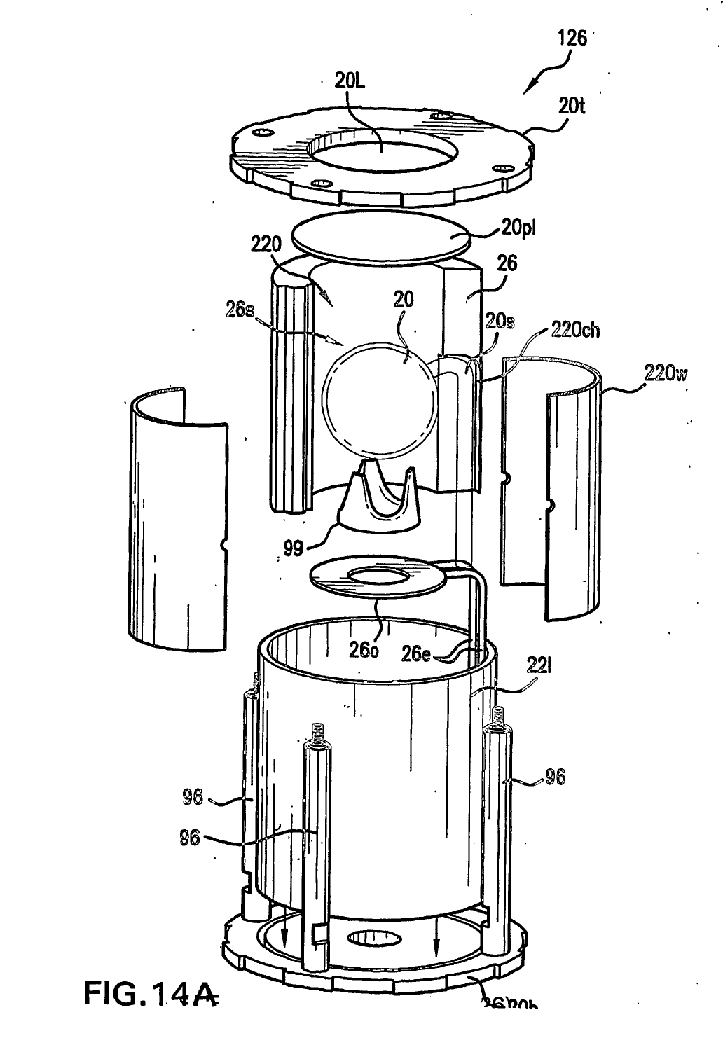

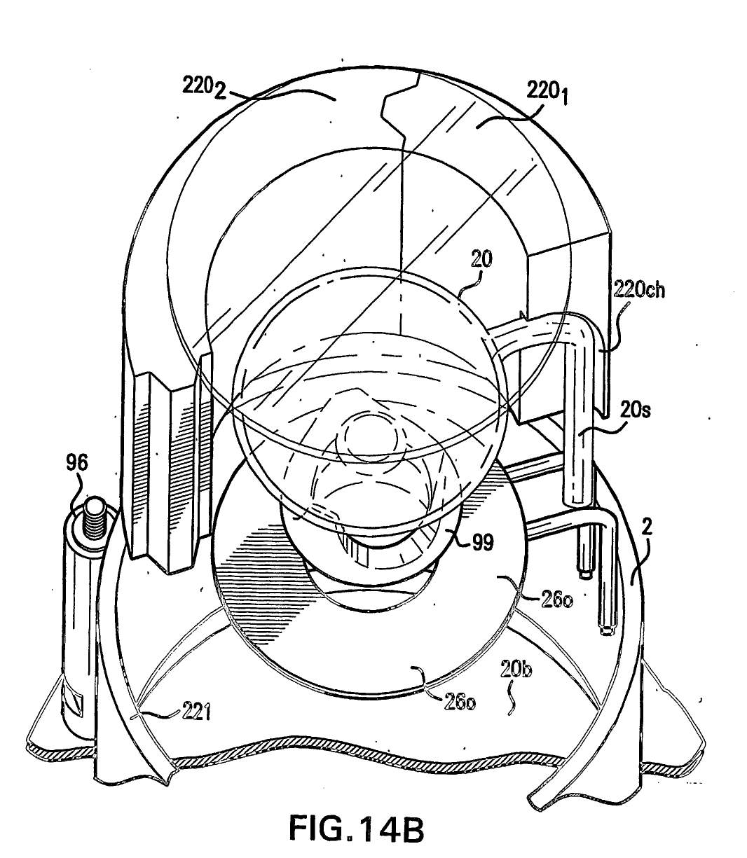

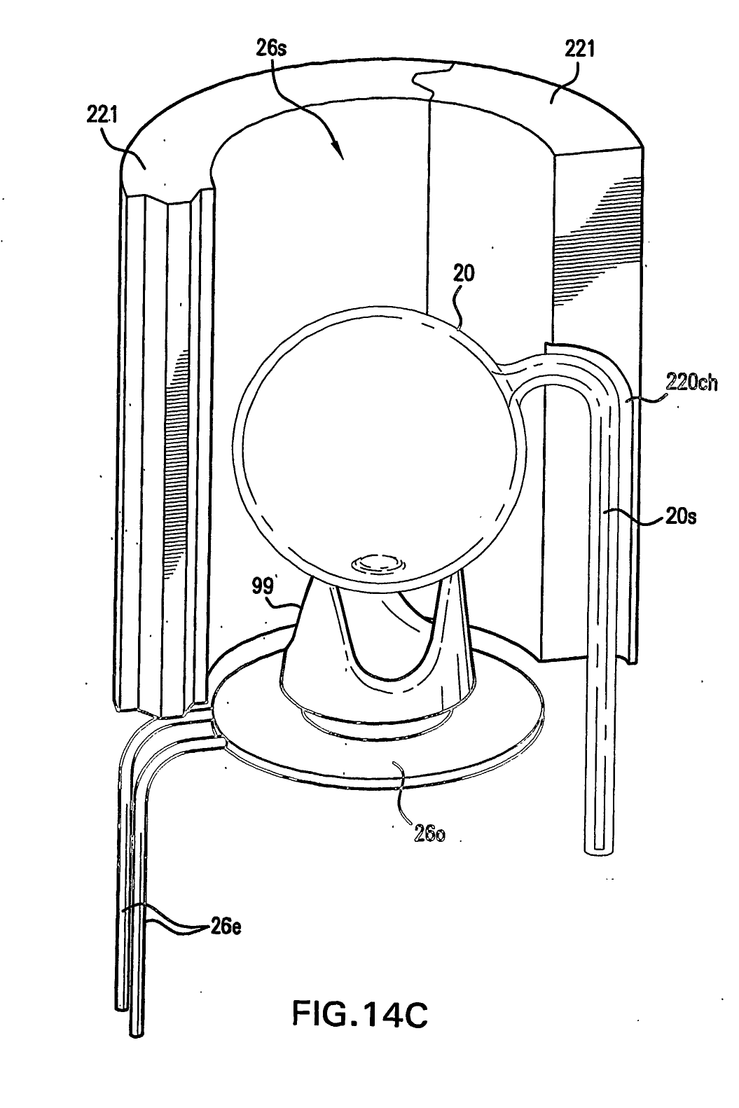

FIGS. 14A-14C illustrate an alternative embodiment of an oven assembly 126 with an optical pumping cell 20 and a housing 221 with upper and lower members 20t, 20b that attach to define an encased thermal space with an axially extending cylindrical insulating member 220 surrounding the optical cell 20. The light port 20L may be closed using a window 20pl that allows the light therein. The insulating member 220 may include a channel 220ch formed into a wall segment to hold the stem of the cell 20s as shown in FIG. 14C. A pair of mating supplemental wall members 220w may be used to further insulate the thermal space 26s provided by the oven assembly 126. As shown, a heating element 26o may be positioned in the housing 221 in communication with the thermal space 26s (shown positioned below the optical pumping cell 20). In the embodiment shown, the heating element 26o is disc shaped. A cup member 99 may be used to hold the cell in desired position in the thermal space 26s. A heating element may not be required as noted above if the thermal space is sufficiently efficient to be able to capture enough energy to provide the desired elevated temperatures. In addition or alternatively, the heating element may be held remotely out of the thermal space and the heat forced into the thermal space 26s. An RF coil 93 may also be positioned on the optical pumping cell 20 (not shown).

FIG. 14B illustrates that the cylindrical insulating member 220 may be formed with interlocking components (shown partially with components 220₁, 220₂) with edge portions that matably attach as shown. FIGS. 14A and 14B show the stem 20s extending down into the oven assembly 126 intermediate the leads 26e of the heating element while FIG. 14C illustrates the stem 20s extending along a path away from the leads 26e.

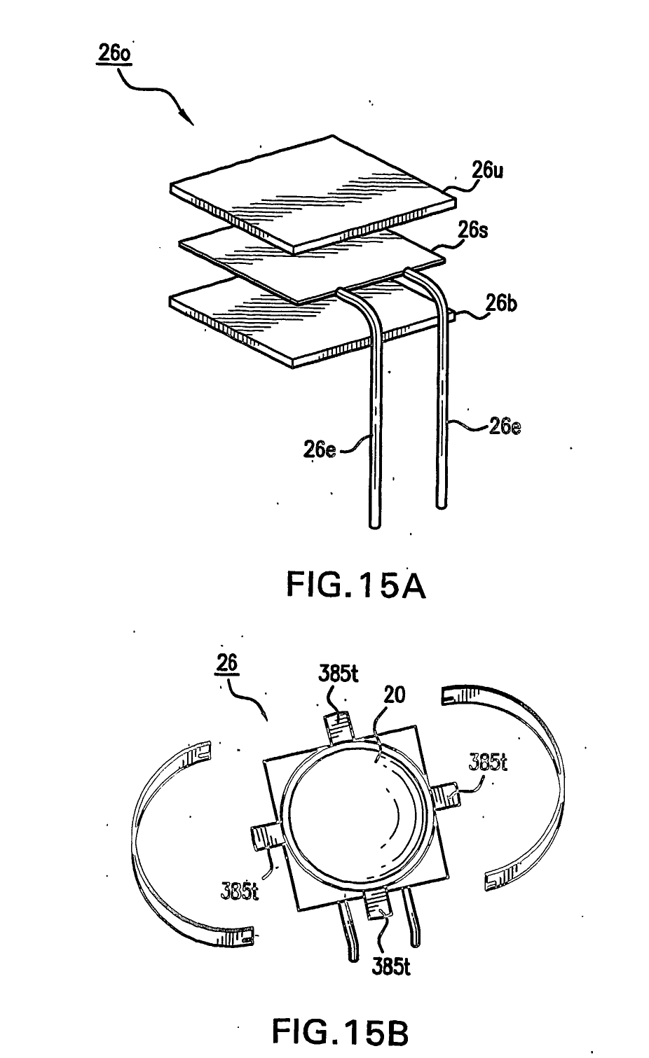

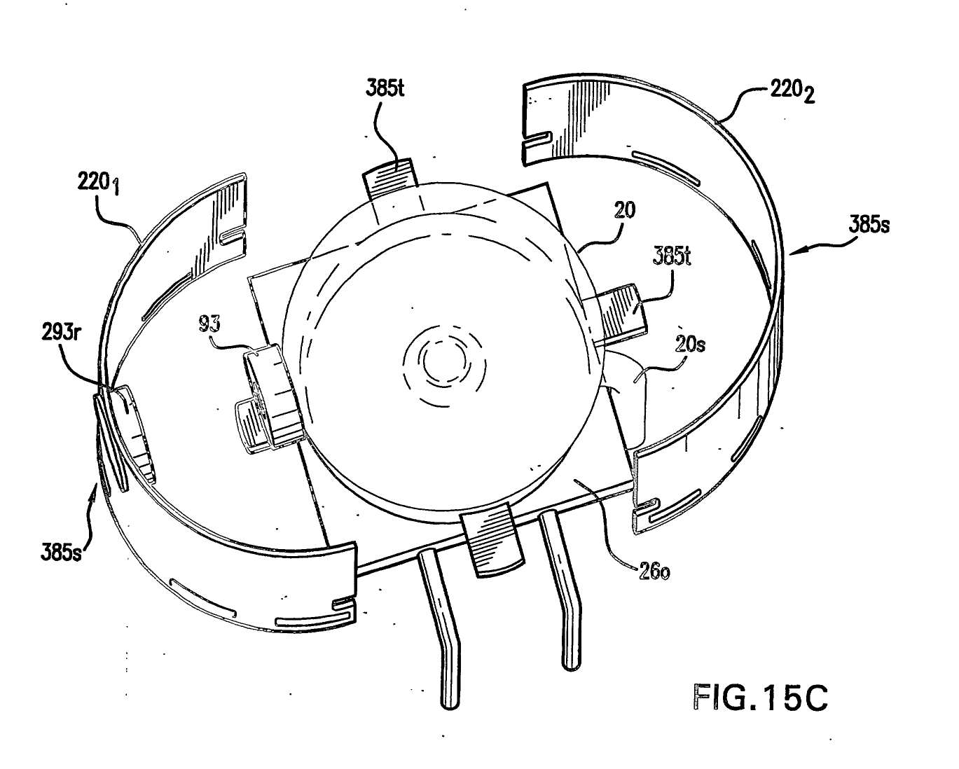

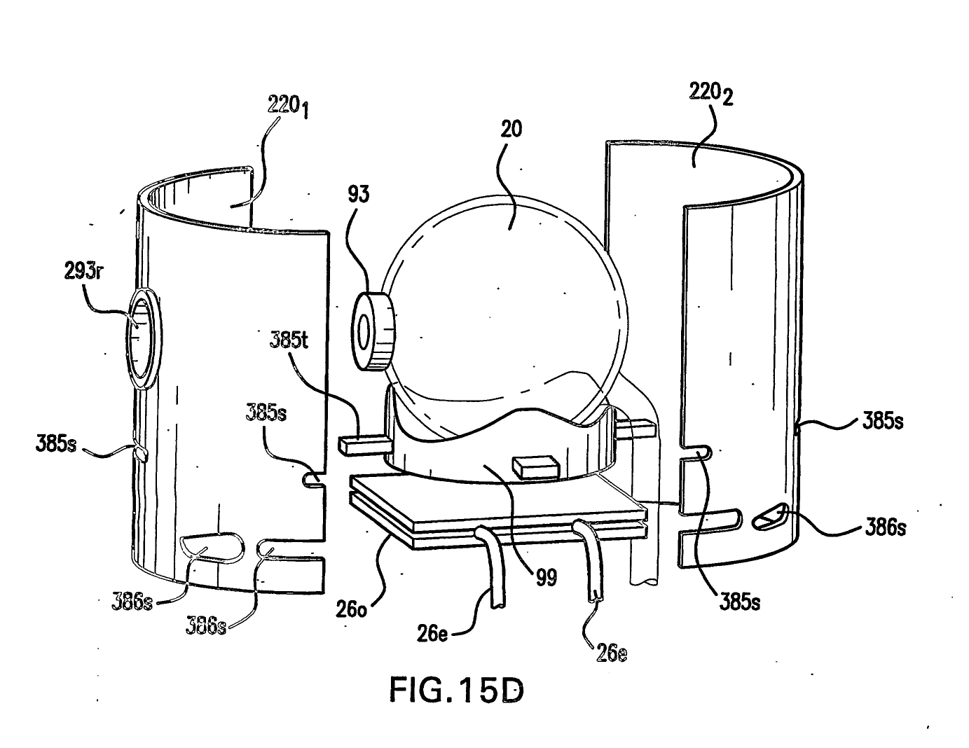

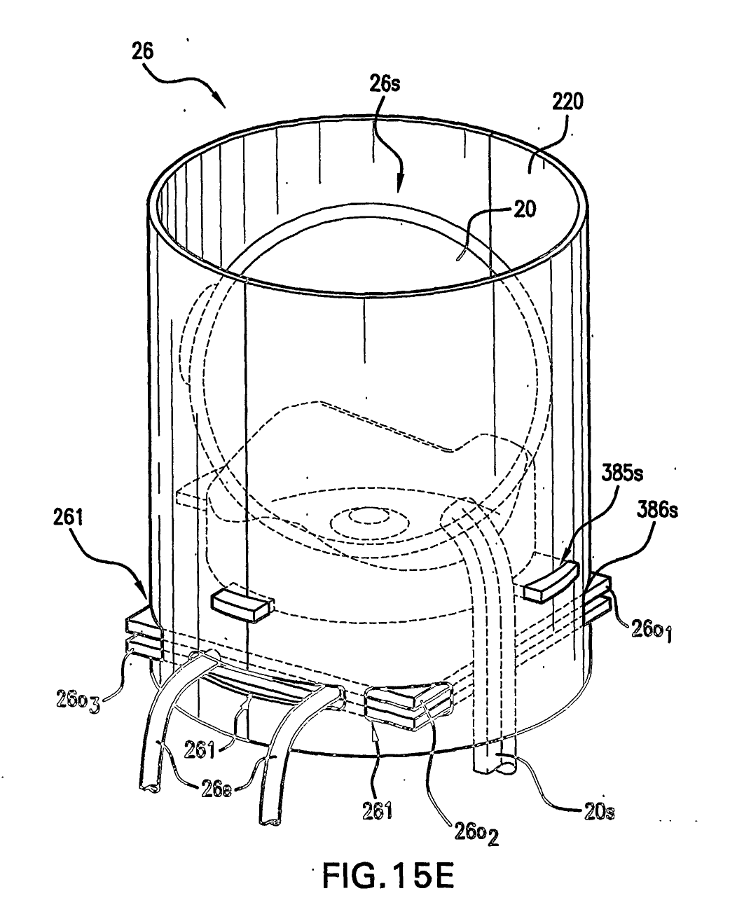

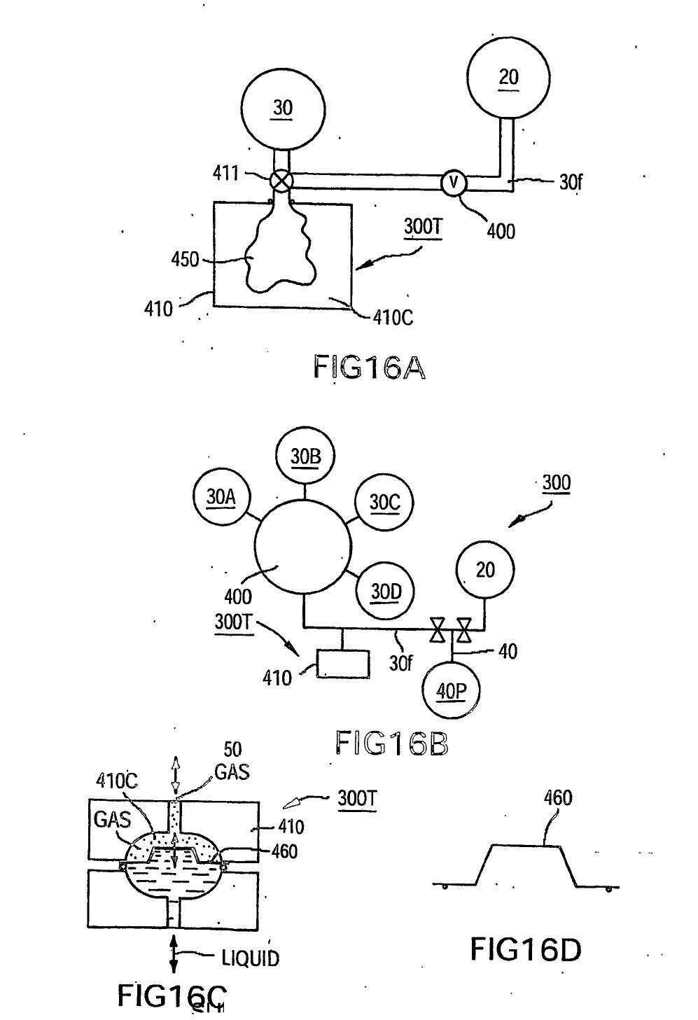

FIG. 15B illustrates yet another example of an oven 26. This oven 26 configuration may be particularly suitable for hypempolarizer configurations using multiple optical pumping cells 20. As shown in FIG. 15A, the oven may include a heating element 26o which has three layers, the upper and lower layers 26u, 26b sandwiching the active heating plate 26s. The thermal insulating housing 220 (FIG. 1SE) encases the cell 20 therein. The cup holder 99 may include tabs 385t that are configured to extend through mating slots 385s in the housing 220. Similarly, the edges 26o₁, 26o₂, 26o₃ (FIG. 15E) of the heater 26o and the leads 26e may extend through corresponding slots 261 in the housing 386s to position both the heating element 26o and the cell 20 above the lowermost portion of the walls of the thermal housing 220. As before, the thermal housing 220 may be formed from interlocking (semi-cylindrical) components 220₁, 220₂. FIG. 15C illustrates that one of the wall segments 220, can be configured with an RF coil recess 293r to hold the NMR coil 93 in position abutting the cell 20.

FIG. 16A illustrates a gas distribution system 300 having selectable flow paths 30f with a gas transfer mechanism 300T that uses pressure differentials to direct target gas between the optical pumping cell 20 and the selected holding cell 30. As shown, the gas distribution valve 400 is in fluid communication with the holding cells 30 (shown for clarity as a single cell) and the polarization or optical pumping cell 20. FIG. 16B illustrates that the gas distribution system 300 uses the gas distribution valve 400 to serially connect or a desired holding cell 30 and connect it to the gas transfer mechanism 300T so as to be able to flow the target gas in a desired direction.

FIG. 16B shows the valve 400 as it connects to each holding cell 30A-30D in a gas distribution system 300 that uses the gas transfer mechanism 300T to direct the target gas to and from the optical pumping cell 20 as well as to mete out or deliver doses of the polarized gas to the dispensing port 40p. Referring again to FIG. 16A, the gas transfer mechanism 300T employs a housing with a pressure chamber 410 and a resilient or compressible member 450. In the embodiment shown, the resilient member 450 is an elastomeric bag, such as a TEDLAR bag, or other bag formed of or coated with materials that can provide a suitable T1 for polarized gas. Valve 411 is optional and may be a glass valve used to isolate the holding cell and/or transfer mechanism 300T. In operation, fluid, typically a liquid such as oil (which may be a non-toxic biodegradeable oil) is directed into the cavity of the pressure chamber 410c. Nitrogen gas may also be suitable. The input of fluid into the chamber compresses the bag 450 and forces the gas in the bag 450 out into the flow path (to the cell 20 or 30). In the reverse, removing the fluid from the chamber acts to evacuate the system and pull the target gas into the bag 450.

The lines in the gas flow path can be formed of small LD. tubing to reduce the dead volume in the lines of the flow path. For example, 0.03 inch PTFE tubing can be suitable to form portions or all of the flow paths. In certain embodiments, the gas transfer mechanism 300T can be used to provide meted volumes of polarized target gas 50p to the dispense port 40p. Using an incompressible liquid such as oil, and knowing the volume, temperature and pressure of the liquid, the volume of target gas dispensed can be calculated. The gas transfer mechanism 300T does not require a motorized pump to operate to transfer the polarized gas, but such a pump may be used to transfer non-polarized fluid (target gas, filler gas, purge gas, and the like).

FIG. 16C illustrates a pressure chamber 410 that employs a membrane 460 that extends across the cavity 410c as the resilient member 450. The membrane 460 is conformable to the shape of the cavity and provides a dividing line, with target gas 50 held on one side and the liquid compression fluid on the other. As liquid is forced into the cavity 410c, the membrane 460 deflects to push out the target gas 50. The membrane 460 may be sized to deflect sufficiently to contact the upper and/or lower walls of the cavity. The upper deflection occurs when sufficient liquid has been introduced into the cavity 410 and the lower deflection occurs when the liquid has been withdrawn, thereby pulling down (or to a side) the membrane 460. The cavity 410c may be sized so that, at full deflection, the membrane 460 and cavity 410c can hold about 1.0 L of target gas therein. Other sizes may also be accommodated. As shown in FIG. 16D, the membrane 460 may be pre-shaped with a ramped projection profile to help push the target gas from the cavity. Other membrane shapes, such as dome shaped membranes may also be useful.

FIGS. 17A-17D illustrate a gas transfer mechanism 300T using a bladder 450b as the resilient member 450. The bladder 450b can include a series of pleats 450p. The pressure chamber 410 includes a lid, a platform 410s with a seal 411, and a primary body 410b. The platform 410s holds the port 474p for the target gas entry and exit and the port 475p for the fluid. The lid 410l secures to the body 410b and compresses the seal, defining the pressure chamber 410. The pressure chamber components are sized and configured to hold the bladder 450b therein and to allow fluid (typically liquid) to controllably enter and exit from the chamber 410 via port 475p and flow path 475 (attached to the fluid source).

Another example of a gas transfer mechanism 300T and exemplary components and operation is described in co-assigned, co-pending U.S. Provisional Patent Application Ser. No. 60/398,033, filed Jul. 23, 2002, the contents of which are hereby incorporated by reference as if recited in full herein.

FIG. 17E illustrates a hyperpolarizer 10 with the gas transfer mechanism 300T positioned below the holding cells 30 and the optical pumping cell 20. The optic system 15 can be housed in an overhead housing 15h and connect to an optical tube 15T that blocks perimeter light and extends between the housing 15h and the light port 20L of the oven 26 to direct the laser light to the cell 20. The optic housing 15h is suspended above the solenoid 80 by a bracket 15b that attaches to an upper portion 15Tu of the optical tube 15T. As shown in FIG. 17F, the pressure chamber 410 of the gas transfer mechanism 300T, the holding cells 30, and the oven with optical cell(s) 20 all extend inside the solenoid cavity 80c within a region of homogeneity “B_{H}”. The solenoid 80 may be end-compensated (with the number of coil wraps being increased on the two opposing end portions relative to the center portion of the solenoid) to increase the length of the region of homogeneity B_{H}, but typically, the region can be approximated as being in the central third of the length of the solenoid 80. A single continuous length of 16 gauge wire can be wrapped to provide a solenoid 80 with about double the number of wrappings on end portions relative to the center portion (which may have a length that is longer than the sum of the lengths of both end portions) to provide a homogeneous region B_{H} that is about 8 inches in diameter and about 18 inches long. FIGS. 18A and 18B illustrate the hyperpolarizer 10 with the components shown in FIGS. 17E and 17F slideably placed and secured inside the cavity of the solenoid 80.

FIG. 18C illustrates the hyperpolarizer 10 inside a housing 500. As shown, the hyperpolarizer 10 also includes a source of high purity purge gas 510 (such as grade 5 Nitrogen) and a vacuum pump 512. The purge gas 510 and vacuum pump 512 are configured to engage the control module 11. The control module 11 may also include a display/user interface 570 and a power supply. FIG. 15D illustrates a housing 500 with a side-by-side cabinet arrangement and a gas dispensing line 40 that axially extends out of the solenoid 80 and then rises to an access region in the housing 500 which positions the dispensing port 40p at a user level. The optic system 15 may be configured to transmit light down into the solenoid cavity 80c as shown. In other embodiments, the solenoid 80 and the internal components can be rearranged to allow the optic system 15 to transmit light to the optical pumping cell or cells 20 in an upward direction (not shown).

FIGS. 19A and 19B illustrate the solenoid 80 in a diagonal orientation that is at a height sufficient to allow the dispensing path 40 to extend primarily within the solenoid 80 and so that the dispensing path 40 is axially aligned with the solenoid 80 with the dispensing port 40p configured to exit relatively close to the end of the solenoid 80. That is, the substantially cylindrical solenoid 80 that generates a magnetic holding field has an elongate cavity and an associated axial center line. The hyperpolarizer 10 shown in FIGS. 19A and 19B orients the solenoid 80 so that the axial center line extends in an angularly offset direction with vertical and horizontal components (primarily in a downward direction, angularly offset from the vertical axis). Typically, the dispensing port 40 resides within about 6-12 inches of the lower end portion of the solenoid 80 and the dispensing path 40 is not required to climb more than about 4 inches.

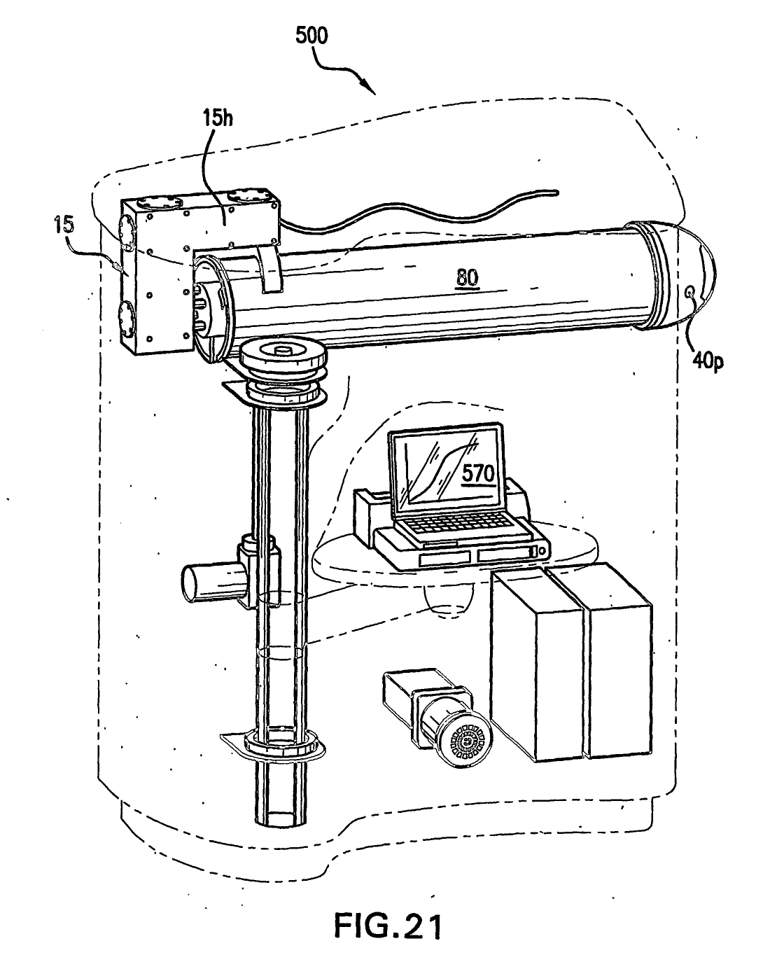

FIG. 21 illustrates that the solenoid 80 may be configured in a substantially horizontal orientation within the hyperpolarizer 10 with the optic system 15 oriented to transmit light substantially horizontally into the solenoid 80. The dispensing port 40p may be configured proximate to the other end of the solenoid 80 or may be directed to output at a different location from the cabinet 500. Other orientations of the solenoid 80 and the polarizing and gas transfer components may also be employed. The envelope of homogeneity may also be increased by using mu metal shielding. For additional discussion of mu metal shielding, see U.S. Pat. No. 6,269,648, the contents of which is hereby incorporated by reference as if recited in full herein.

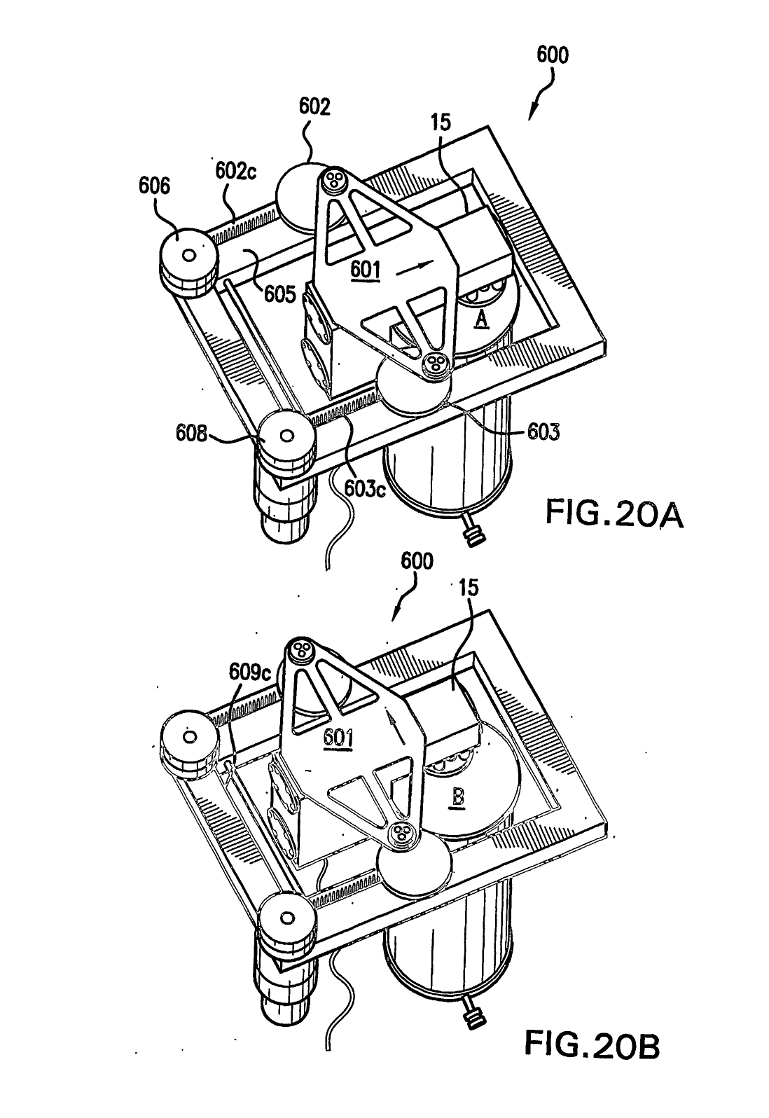

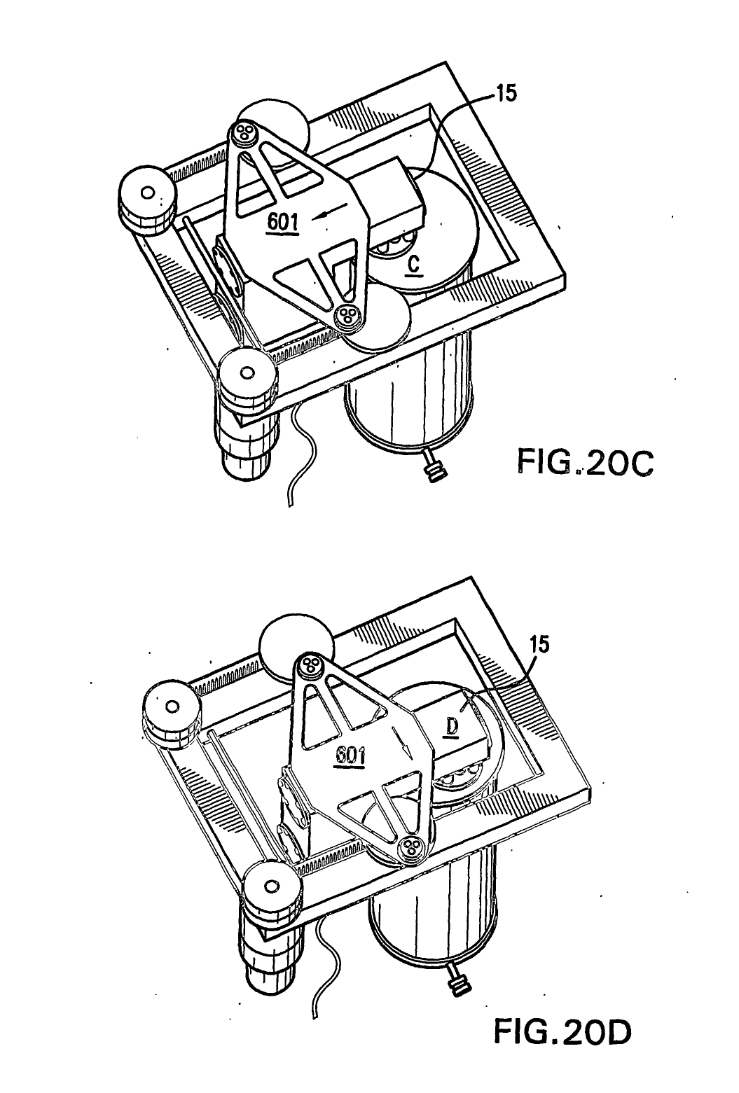

FIGS. 19A and 19B also illustrate that the optic system 15 can be operably associated with a drive system 600 that translates at least portions of the optic system 15 to direct the laser light to selected locations in the solenoid cavity to selectively polarize target gas held in one or more of the optical pumping cells 20. FIGS. 20A-20D illustrate four different locations (shown as positions A, B, C, D) that the optic system housing 15h can move through to position the laser light over regions with respect to the solenoid 80. As shown, the drive system 600 includes a primary bracket body 601 that resides above and is secured to the housing 15h. The bracket 601 is attached to rotatable wheels 602, 603 positioned on opposing sides of a frame 605. Drive wheels 606, 608 connect with drive links 609c, 602c, 603c that rotate the wheels 602, 603 which cooperate to move the bracket 601 forward, rearward, left or right, which, in turn, moves the optic head and positions the optic system housing 15h at a desired location. The drive links 602c, 603c, 609c can be any desired configuration such as, but not limited to, belts, chains, or the like. The controller 11 can be configured to automatically translate the optic system 15 to the desired location to repolarize or originally polarize the selected cell 20.

The display and/or user interface or input means 570 can include a monitor as well as a keyboard, touch screen or the like that can allow an operator to input a dispense request. In other embodiments, the user interface can be configured to allow remote input of the scheduling via a computer network, whether local, regional, national (intranet) or global (internet). The display or interface 570 can also display or relay information regarding the operational status and function of the hyperpolarizer 10 such as the polarization level of the gas in the optical pumping cell 20 or holding cells 30 or any detected operational errors or discrepancies during operation.

As will be understood by those of skill in the art, in certain embodiments, with reference to FIG. 18C, the controller 11 is configured to provide the purge/pump capacity from a central purge gas source 510 and vacuum pump 512 to the gas flow paths or channels to clean the system of contaminants. As such, fluid flow paths of plumbing extending between the purge and vacuum sources to the optical pumping cell(s) 20 and/or holding cells 30 can be defined by a fluid distribution system or manifold network of plumbing, valves, and solenoids. These fluid flow paths selectively direct purge gas to and from the optical pumping cells 20, holding cells 30, dispensing path 40, and/or gas transfer mechanism 300T to purge and evacuate the flow paths in order to prepare them for polarization operations or to hold or process polarized gas.

The quantity of target gas can be sized so as to provide the constituents commensurate with that needed to form a single batch. The unpolarized target gas can be a gas mixture that comprises a quantity of target noble gas and a quantity of one or more high purity biocompatible filler gases. For example, for ³He polarization, an unpolarized gas blend of ³He/N₂ can be about 99.25/0.75. For producing hyperpolarized ¹²⁹Xe, the pre-mixed unpolarized gas mixture can be about 85-98% He (preferably about 85-89% He), about 5% or less ¹²⁹Xe, and about 1-10% N₂ (preferably about 6-10%).

The amount of unpolarized gas mixture in the cell (holding and/or optical pumping cell) can be meted out and configured and sized so that the single batch production run quantity provides a single patient amount for a single MRI imaging or NMR evaluation session. To provide the pharmaceutical grade polarized gas doses, the polarized gas itself may be mixed with pharmaceutical grade carrier gases or liquids upon dispensing, or may be configured to be administered as the only or primary substance or constituent. In particular embodiments, the polarized gas is ³He and is mixed with nitrogen filler gas prior to or during dispensing (or before administration to a patient) to form a volume of gas blend to be inhaled by the patient. In other embodiments, for example, for producing inhalable ¹²⁹Xe, the ¹²⁹Xe may form a major portion (or all) of the administered dose. In other embodiments, the polarized gas can be formulated to be injected in vivo (in a liquid carrier, in microbubble solution, or in gaseous form).

The hyperpolarizer 10 can include one or more purifiers or filters (not shown) that are positioned in line with the plumbing to remove impurities such as water vapor, alkali metal, and oxygen from the system (or to inhibit their entry therein). The hyperpolarizer 10 can also include various sensors including, a flow meter, as well as a plurality of valves, electrical solenoids, hydraulic, or pneumatic actuators that can be controlled by the controller 11 to define the fluid flow path and operation of the components of the hyperpolarizer 10. As will be understood by those of skill in the art, other flow control mechanisms and devices (mechanical and electronic) may be used within the scope of the present invention.

The optical cell 20 can also employ helium as a buffer gas to pressure-broaden the alkali metal (typically Rb) vapor absorption bandwidth. The selection of a buffer gas is important because the buffer gas—while broadening the absorption bandwidth—can also undesirably impact the alkali metal-noble gas spin-exchange by potentially introducing an angular momentum loss of the alkali metal to the buffer gas rather than to the noble gas as desired.

As will be appreciated by those of skill in the art, Rb is reactive with H₂O. Therefore, any water or water vapor introduced into the polarizer cell 130 can cause the Rb to lose laser absorption and decrease the amount or efficiency of the spin-exchange in the polarizer cell 130. Thus, as an additional precaution, an extra filter or purifier (not shown) can be positioned before the inlet of the polarizer cell 130 with extra surface area to remove even additional amounts of this undesirable impurity in order to further increase the efficiency of the hyperpolarizer 10.

In any event, once the polarization process is complete, polarized gas exits the optical pumping cell 20 (and, if used, to the holding cell 30) and is ultimately directed to gas dispensing system 40 and then to a collection or accumulation container such as a patient delivery container or drug container. For additional descriptions of meted dispensing systems, see co-pending U.S. patent application Ser. Nos. 10/277,911 and 10/277,909 and U.S. Provisional Application Ser. No. 60/398,033, the contents of which are hereby incorporated by reference as if recited in full herein.

As described above, forced cooling can be used to actively rapid cool the polarization chamber. As such, the hyperpolarizer unit 10 can also include a cooling source in fluid communication with the oven and/or cell to cool the optical pumping cell 20 and/or oven 26 after the polarization process. The cooling source can include a refrigeration source that can turn the oven 26 into a cooling chamber to precipitate the alkali metal from the polarized gas stream. In other embodiments, heat to the oven 26 is turned off and natural cooling is used to condense the Rb from the vapor phase and collect it in the bottom of the optical pumping cell 20. In addition, a micro-pore filter can be positioned in the gas dispensing line or in the exit flow path (extending between the optical cell exit port to the dispensing port).

A delivery or receiving container such as a patient dose bag or other vessel can be attached to the dispensing outlet 40p. A valve or other device located thereat can be opened to evacuate the attached bag or other delivery vessel. Once the bag is evacuated, the polarized gas can be directed into the bag directly or into a mixing/blending chamber (not shown) where a high-grade biocompatible filler gas can be added as desired in a desired blend formulation.

In certain embodiments, the blending is performed in situ corresponding to the scheduled procedure (and its associated gas formulation) and/or the polarization level of the gas. That is, the hyperpolarizer 10 can be configured with a mixing/blending chamber and a source of biocompatible fluid that will be combined with the polarized gas to provide the blended formulation of pharmaceutical polarized gas product proximate in time and at the production site of the polarized gas itself.

In other embodiments, the receiving container can be pre-filled with a high purity medical grade holding gas such as N₂ to inhibit the permeation of oxygen therein. The holding gas can form part of the blended formulation or can be expelled prior to dispensing the polarized gas or gas mixture.

In certain particular embodiments, a polarization measurement is obtained and a formulated blend volume of unpolarized gas added or dispensed separately or in combination with the polarized gas based on the polarization level to form a controlled blend for more consistent imaging/NMR evaluations procedure to procedure. The blending may be carried out automatically by the hyperpolarizer 10 by controlling the amount of polarized gas and the amount of fluid blending constituent(s) that are released the dispensing container to provide the formulated blend. For additional description of optical pumping modules, systems, and blending methods, see co-assigned U.S. application Ser. No. 10/277,909, the contents of which are hereby incorporated by reference as if recited in full herein. See also, U.S. patent application Ser. No. 09/949,394 for descriptions of methods and devices for providing meted formulations and amounts of polarized gas, the contents of which are hereby incorporated by reference as if recited in full herein.

The hyperpolarizer 10 can be located at the point of use site (hospital or clinic) typically in the vicinity of or proximate to the MRI or NMR equipment. That is, the hyperpolarizer 10 can reside adjacent the MRI suite or in a room of a wing proximate thereto so as to limit the spatial transport and potential exposure to undesirable environmental conditions. In certain embodiments, the polarized gas transport time between the hyperpolarizer and the imaging suite is less than about 1 hour. Placing the hyperpolarizer in the clinic or hospital allows for short and consistent transport times procedure to procedure. In addition, formulating the pharmaceutical polarized gas with a polarized gas having higher levels of polarization can reduce the amount of the polarized gas used to form the end dose product, thereby potentially reducing the cost of the product.

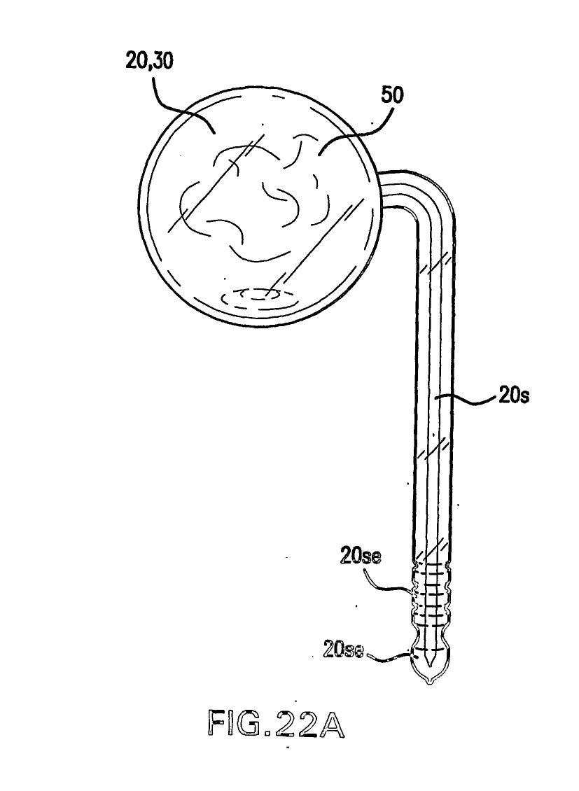

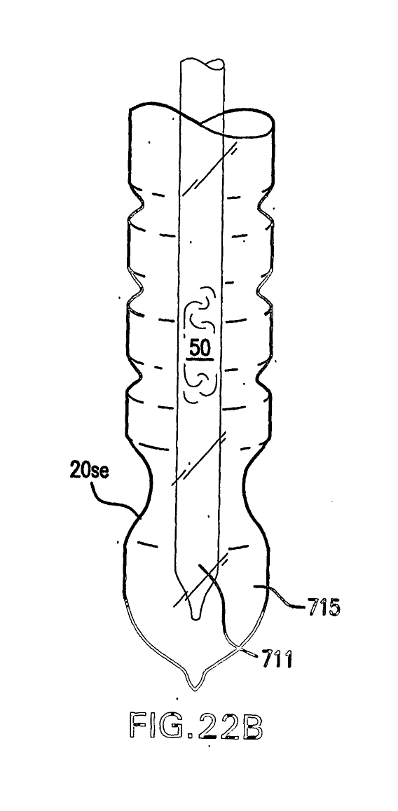

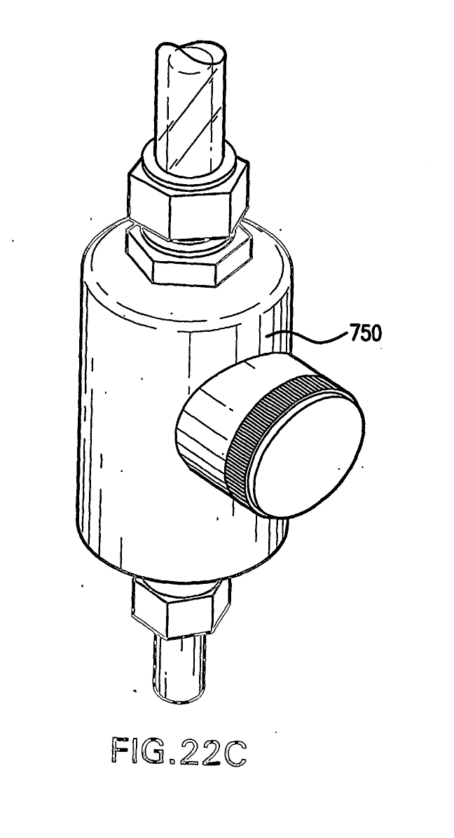

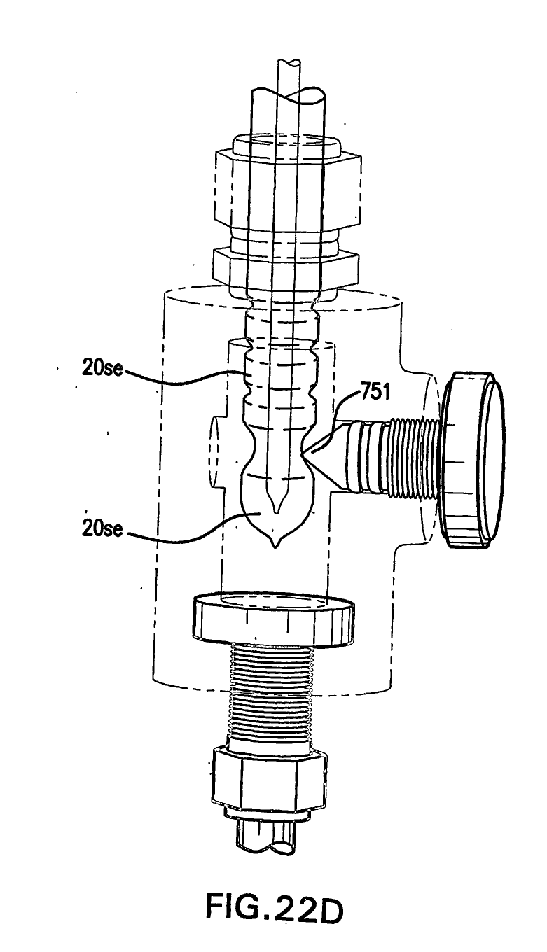

FIGS. 22A and 22B illustrate exemplary configurations of a cell 20 according to embodiments of the present invention. As shown, the cell 20 includes a sealed stem end portion 20se. FIG. 22B is an enlarged view of the sealed stem portion 20se. As shown, a small inner flow channel 711 is sealed within a protective outer shell or wall 715. The cell 20 may be pre-filled with a target gas 50 (and/or alkali metal as needed). As shown in FIG. 22D, the stem 20s can be inserted into a valve 750 that has a break edge point 751 that can be advanced against and then break the seal of the shell or wall 715 at the lower edge portion 20se releasing gas (if any) from the channel 711 in a captured space within the valve body and inhibiting oxygen contamination. The valve 750 and cell 20 can form a portion of the gas flow path in the gas distribution system. The valve 750 may further be connected in fluid communication with relatively flexible or relatively rigid tubing or conduits. This cell configuration and discussion is applicable to cell 30 with stem 30s.

It is noted that polarimetry systems are well known to those of skill in the art. The polarization strength of the polarized gas can be monitored using polarimetry and the RF NMR surface polarimetry coil 93. See, e.g., U.S. Pat. No. 6,295,834 and U.S. patent application Ser. No. 09/334,341, and Saam et al., Low Frequency NMR Polarimeter for Hyperpolarized Gases, Jnl. of Magnetic Resonance 134, 67-71 (1998), the contents of which are hereby incorporated by reference as if recited in full herein.

As will be appreciated by one of skill in the art, the present invention may be embodied as a method, data or signal processing system, or computer program product. Accordingly, the present invention may take the form of an entirely hardware embodiment, an entirely software embodiment or an embodiment combining software and hardware aspects. Furthermore, the present invention may take the form of a computer program product on a computer-usable storage medium having computer-usable program code means embodied in the medium. Any suitable computer readable medium may be utilized including hard disks, CD-ROMs, optical storage devices, or magnetic storage devices.

The computer-usable or computer-readable medium may be, for example but not limited to, an electronic, magnetic, optical, electromagnetic, infrared, or semiconductor system, apparatus, device, or propagation medium. More specific examples (a nonexhaustive list) of the computer-readable medium would include the following: an electrical connection having one or more wires, a portable computer diskette, a random access memory (RAM), a read-only memory (ROM), an erasable programmable read-only memory (EPROM or Flash memory), an optical fiber, and a portable compact disc read-only memory (CD-ROM). Note that the computer-usable or computer-readable medium could even be paper or another suitable medium upon which the program is printed, as the program can be electronically captured, via, for instance, optical scanning of the paper or other medium, then compiled, interpreted or otherwise processed in a suitable manner if necessary, and then stored in a computer memory.

Computer program code for carrying out operations of the present invention may be written in an object oriented programming language such as Java7, Smalltalk, Python, or C++. However, the computer program code for carrying out operations of the present invention may also be written in conventional procedural programming languages, such as the “C” programming language or even assembly language. The program code may execute entirely on the user's computer, partly on the user's computer, as a stand-alone software package, partly on the users computer and partly on a remote computer or entirely on the remote computer. In the latter scenario, the remote computer may be connected to the users computer through a local area network (LAN) or a wide area network (WAN), or the connection may be made to an external computer (for example, through the Internet using an Internet Service Provider).

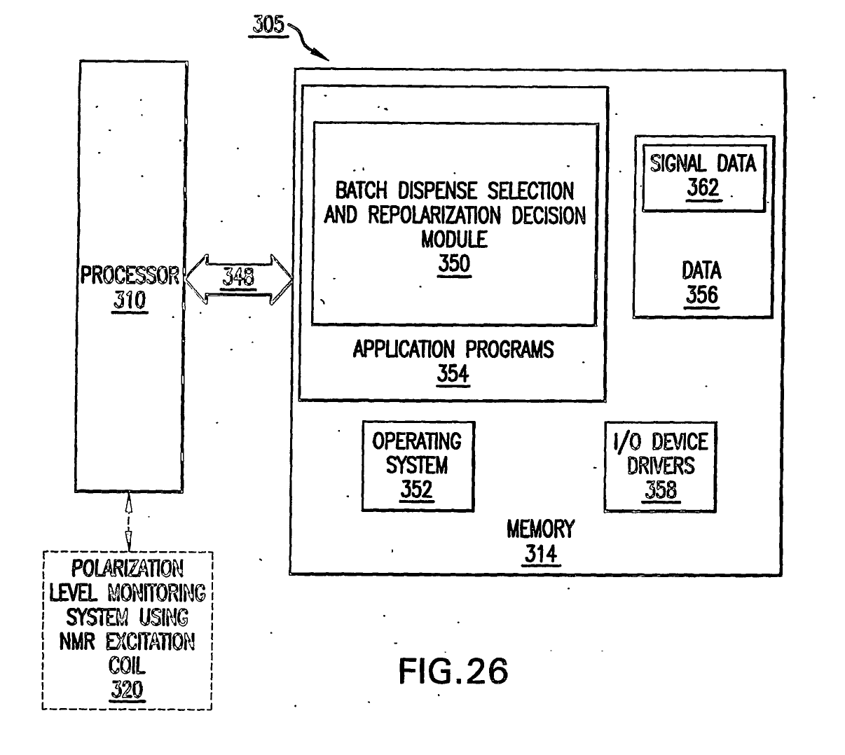

FIG. 26 is a block diagram of exemplary embodiments of data processing systems that illustrates systems, methods, and computer program products in accordance with embodiments of the present invention. The processor 310 communicates with the memory 314 via an address/data bus 348. The processor 310 can be any commercially available or custom microprocessor. The memory 314 is representative of the overall hierarchy of memory devices containing the software and data used to implement the functionality of the data processing system 305. The memory 314 can include, but is not limited to, the following types of devices: cache, ROM, PROM, EPROM, EEPROM, flash memory, SRAM, and DRAM.

As shown in FIG. 26, the memory 314 may include several categories of software and data used in the data processing system 305: the operating system 352; the application programs 354; the input/output (I/O) device drivers 358; a Batch Selection Module for Polarization, Repolarization, and Dispensing 350; and the data 356. The application programs 354 and/or Batch Selection Module 350 can include software and/or data that directs the operational sequences, provides the control logic and/or computational algorithms, and the like. The data 356 may include image data 362 which may be obtained from an NMR coil and/or NMR polarimetry system 320. As will be appreciated by those of skill in the art, the operating system 352 may be any operating system suitable for use with a data processing system, such as OS/2, AIX or OS/390 from International Business Machines Corporation, Armonk, N.Y., WindowsXP, WindowsCE, WindowsNT, Windows95, Windows98 or Windows2000 from Microsoft Corporation, Redmond, Wash., PalmOS from Palm, Inc., MacOS from Apple Computer, UNIX, FreeBSD, or Linux, proprietary operating systems or dedicated operating systems, for example, for embedded data processing systems.

The I/O device drivers 358 typically include software routines accessed through the operating system 352 by the application programs 354 to communicate with devices such as I/O data port(s), data storage 356 and certain memory 314 components and/or the image acquisition system 320. The application programs 354 are illustrative of the programs that implement the various features of the data processing system 305 and preferably include at least one application that supports operations according to embodiments of the present invention. Finally, the data 356 represents the static and dynamic data used by the application programs 354, the operating system 352, the I/O device drivers 353, and other software programs that may reside in the memory 314.

While the present invention is illustrated, for example, with reference to the background estimator module 350 being an application program in FIG. 26, as will be appreciated by those of skill in the art, other configurations may also be utilized while still benefiting from the teachings of the present invention. For example, the Batch Dispense Selection and Re-polarization Module 350 may also be incorporated into the operating system 352, the I/O device drivers 358 or other such logical division of the data processing system 305. Thus, the present invention should not be construed as limited to the configuration of FIG. 26, which is intended to encompass any configuration capable of carrying out the operations described herein.

In certain embodiments, the Batch Dispense Selection and Repolarization Decision Module 350 includes computer program code for tracking polarization level data of a plurality of batches of target gas, identifying when a batch has decayed sufficiently that it is ready to be repolarized or is not suitable for use, and/or identifying which of a plurality of polarized gas batches that are already polarized will be dispensed upon an output request from a user. The dispense selection can be based on a dynamic reading and/or analysis of the polarization level in the individually and controllably selectable various batches. The Module 350 can direct initiation of operations that will automatically determine whether and when to repolarize individual batches of target gas and initiate controller operations that will do one or more of the following: (a) engage the optic system with the appropriate pumping cell; or (b) release the gas from a holding cell and direct it to the pumping cell. If the former, the engagement can be carried out by one or combinations of: (a) translating one or more of the optical pumping cells with respective batches of target gas therein to align with a static optic system; (b) altering (such as redirecting) the optic laser beam path in the optic system to align with the desired optical pumping cell(s); and (c) translating the optic system to optically engage with the selected pumping cell(s). The Module 350 can be configured to track the decay of each batch of target gas in their respective cell over time to be able to supply suitably polarized target gas to a user “on-demand” by selecting one or more of the polarized gas batches held for use.

The I/O data port can be used to transfer information between the data processing system 305 and the NMR data acquisition system 320 or another computer system, a network (e.g., the Internet) or other device controlled by the processor. These components may be conventional components such as those used in many conventional data processing systems, which may be configured in accordance with the present invention to operate as described herein.

While the present invention is illustrated, for example, with reference to particular divisions of programs, functions and memories, the present invention should not be construed as limited to such logical divisions. Thus, the present invention should not be construed as limited to the configuration of FIG. 26 but is intended to encompass any configuration capable of carrying out the operations described herein.

The flowcharts and block diagrams of certain of the figures herein illustrate the architecture, functionality, and operation of possible implementations of probe cell estimation means according to the present invention. In this regard, each block in the flow charts or block diagrams represents a module, segment, or portion of code, which comprises one or more executable instructions for implementing the specified logical function(s). Certain of the flowcharts and block diagrams illustrate methods to operate hyperpolarizers or components thereof to yield polarized gas according to embodiments of the present invention. In this regard, each block in the flow charts or block diagrams represents a module, segment, or portion of code, which comprises one or more executable instructions for implementing the specified logical function(s). It should also be noted that in some alternative implementations, the functions noted in the blocks may occur out of the order noted in the figures. For example, two blocks shown in succession may in fact be executed substantially concurrently or the blocks may sometimes be executed in the reverse order, depending upon the functionality involved.

The foregoing is illustrative of the present invention and is not to be construed as limiting thereof. Although a few exemplary embodiments of this invention have been described, those skilled in the art will readily appreciate that many modifications are possible in the exemplary embodiments without materially departing from the novel teachings and advantages of this invention. Accordingly, all such modifications are intended to be included within the scope of this invention as defined in the claims. In the claims, means-plus-function clauses, where used, are intended to cover the structures described herein as performing the recited function and not only structural equivalents but also equivalent structures. Therefore, it is to be understood that the foregoing is illustrative of the present invention and is not to be construed as limited to the specific embodiments disclosed, and that modifications to the disclosed embodiments, as well as other embodiments, are intended to be included within the scope of the appended claims. The invention is defined by the following claims, with equivalents of the claims to be included therein.

1. A method for producing hyperpolarized gas, comprising the steps of:
providing a plurality of cells, each having a respective quantity of target gas held therein;
polarizing the target gas in and/or from the cells in a desired order to provide separate batches of polarized gas; and
repolarizing the previously polarized target gas held in at least one of the cells when the polarization level falls below a predetermined value.

2. A method according to claim 1, wherein the polarizing and/or repolarizing steps are carried out to serially polarize gas in and/or from selected cells.

3. A method according to claim 1, wherein the polarizing and/or repolarizing steps are carried out to concurrently polarize gas from or in at least two cells.

4. A method according to claim 1, further comprising the step of monitoring the polarization level of the batches of polarized target gas in the cells during a monitoring period;
and wherein said polarizing and repolarizing steps are carried out so that the polarized target gas in at least one of the cells has a different polarization decay cycle relative to the polarized target gas in at least one of the other cells during a monitoring period.

5. A method according to claim 1, wherein said repolarizing step further comprises automatically sequencing the order and time in which a batch of previously polarized target gas is repolarized so that, at full operational status, the hyperpolarizer is adapted to hold a plurality of different batches of polarized target gas, each with a different polarization level and/or respective polarization decay cycle.

6. A method according to claim 1, wherein said polarizing step is carried out using a single optic system with a laser source that is configured to generate circularly polarized light.

7. A method according to claim 1, wherein said polarizing step comprises optically pumping the target gas with circularly polarized light generated by an optic system, wherein the plurality of cells comprises a plurality of optical pumping cells, each configured to be selectively positioned to be in optical communication with the optic system during the polarizing and/or repolarizing steps.

8. A method according to claim 1, wherein the plurality of cells comprises one optical pumping cell and a plurality of holding cells in fluid communication with the optical pumping cell.

9. A method according to claim 8, wherein said polarizing step further comprises directing target gas from a selected holding cell to the optical pumping cell in which the target gas is polarized, then re-directing the polarized target gas from the optical pumping cell to the respective holding cell where the polarization level is monitored over time.

10. A method according to claim 9, wherein said repolarizing step further comprises automatically releasing the previously polarized target gas from a selected holding cell so that the released target gas flows to the optical pumping cell for repolarization, then returns to the respective holding cell.

11. A method according to claim 9, further comprising the step of automatically sequencing the order and time in which a batch of previously polarized target gas is released to be repolarized so that a plurality of different batches of polarized target gas is provided, the automatic release being based on whether the value of the monitored polarization level of a batch or batches is below a predetermined threshold value, and wherein, in operation, at least one batch of polarized target gas has a clinically suitable polarization level for dispensing upon a request by a user.

12. A method according to claim 10, further comprising the step of employing a valve and manifold system in fluid communication with the optical pumping cell and the holding cells to define respective enclosed gas travel paths that can be controllably, individually and automatically selected.

13. A method according to claim 12, further comprising the step of employing a gas transfer mechanism that can provide a pressure differential in the manifold system to direct the target gas to travel in the desired travel path.

14. A method according to claim 13, wherein the gas transfer mechanism comprises a compressible resilient member in a pressure chamber having a cavity that is in fluid communication with the enclosed gas travel paths.

15. A method according to claim 14, wherein the resilient member is a compressible container held in the pressure chamber, the compressible container being configured to receive and expel the target gas during operation.

16. A method according to claim 14, wherein the resilient member is a membrane that extends across the pressure member cavity, and wherein, in operation, the membrane is configured to expand in opposing first and second directions responsive to receiving and expelling the target gas.

17. A method according to claim 7, further comprising the step of translating the optical pumping cells such that any may be in optical communication with the optic system during the polarizing and/or repolarizing steps.

18. A method according to claim 17, wherein said translating step further comprises rotating the optical pumping cells along a predetermined endless path.

19. A method according to claim 7, further comprising the step of translating the optic system so that the light source is in optical communication with selected optical pumping cells during the polarizing and/or repolarizing steps.

20. A method according to claim 7, further comprising the step of directing the light source to travel to selected locations so that the light source is in optical communication with selected optical pumping cells during the polarizing and/or repolarizing steps.

21. A method according to claim 1, further comprising the step of aligning the cells so that a plurality of the cells are held in a common, substantially horizontal plane under the optic system.

22. A method according to claim 1, wherein the cells have substantially spherical bodies with elongated capillary stems that are configured to allow the target gas to flow therethrough.

23. A method according to claim 22, wherein the elongated capillary stem comprises a linear segment that is substantially straight and an arcuate segment positioned between the linear segment and the spherical body.

24. A method according to claim 1, wherein the plurality of cells is at least two cells.

25. A method according to claim 1, wherein the target gas is ³He.

26. A method according to claim 25, wherein the polarized target gas further comprises a non-polarized buffer or blending gas.

27. A method according to claim 7, wherein the polarizing step comprises concurrently optically pumping a plurality of the optical pumping cells.

28. A method according to claim 24, wherein the number of cells is at least four cells, and wherein the monitoring step comprises positioning a NMR coil proximate to each of the at least four cells and transmitting an excitation pulse and receiving a signal response providing polarization level data in response thereto, said method further comprising comparing the polarization level data of the monitored batches of polarized target gas in the cells and selectively dispensing the polarized target gas in the cell that is determined to be at a suitable polarization level proximate in time to a user's request for polarized gas.

29. A method according to claim 1, further comprising generating a magnetic holding field that has a length, width, and depth sufficient to encase the cells.

30. A method according to claim 1, further comprising the step of providing a substantially cylindrical solenoid with a holding cavity and an axial line extending therethrough, the solenoid being configured to generate the magnetic holding field.

31. A method according to claim 27, wherein the solenoid is configured to generate a low field strength magnetic field.

32. A method according to claim 28, further comprising orienting the solenoid so that it is angularly offset from a vertical axis.

33. A method according to claim 28, further comprising orienting the solenoid so that it is substantially vertical.

34-106. (canceled)
